(19) United States
(12) Patent Application Publication
(10) Pub. No.: US 2023/0390087 A1
(43) Pub. Date: Dec. 7, 2023

(54) Magnetomicrometric Advances in Robotic Control

(71) Applicant: Massachusetts Institute of Technology, Cambridge, MA (US)

(72) Inventors: Hugh M. Herr, Concord, NH (US); Cameron Roy Taylor, Wayzata, MN (US); Seong Ho Yeon, Cambridge, MA (US); Michael Thomas Nawrot, Cambridge, MA (US)

(21) Appl. No.: 18/250,189

(22) PCT Filed: Oct. 21, 2021
(86) PCT No.: PCT/US2021/056092

§ 371 Date: Apr. 21, 2023

Related U.S. Application Data
(60) Provisional application No. 63/104,942, filed on Oct. 23, 2020.

(51) Int. Cl. A61F 2/70; A61B 5/06; A61B 5/11; A61B 5/22; A61B 5/00

(52) U.S. Cl. CPC A61F 2/70; A61B 5/062; A61B 5/1107; A61B 5/224; A61B 5/4523; A61B 5/4528; A61B 5/6811; A61B 2560/0223; A61F 2002/704

(57) ABSTRACT

Systems and methods relating to magnetomicrometry and magnetic-target-based mechanomyography are provided. A method of detecting muscle activation, includes with a magnetic field sensor, detecting lateral vibration of a target implanted at a muscle or a tendon and estimating a level of muscle activation based on the detected lateral vibration. The target comprises a magnetic material.

### RELATED APPLICATION(S)

This application claims the benefit of U.S. Provisional Application No. 63/104,942, filed on Oct. 23, 2020. The entire teachings of the above application are incorporated herein by reference.

### BACKGROUND

Target tracking is useful across a wide range of disciplines and at various scales, including, for example, tissue monitoring, beam bending, fluid dynamics, human-computer interaction, and traffic.

There are many methods to track visually-obscured objects. Magnetic-target tracking has the advantages of being low-cost, portable, and safe. However, current magnet tracking technologies are slow, precluding high-speed real-time magnetic-target tracking. There exists a need for improved magnet tracking methods, as can be used, for example, in tissue monitoring applications.

### SUMMARY

Methods and devices are disclosed that relate generally to magnetic targets and associated sensors and processes for use, for example, in magnetomicrometry and mechanomyography.

### Implantable Targets and Insertion Devices

An implantable target for magnet tracking includes a base structure and a shell structure. The base structure comprises a magnetic or magnetizable material, and the shell structure comprises layers of nickel, copper, gold, and parylene C.

An insertion device for an implantable target includes a cannula, a cartridge, and a pushrod. The cartridge is configured to position an implantable target at the cannula, and the pushrod is receivable in the cannula and configured to push the implantable target from the cartridge through the cannula for delivery to a tissue. The cannula and the pushrod comprise a nonmagnetic material.

An insertion system can include a plurality of implantable targets and an insertion device. The insertion system can provide for delivery of the implantable targets.

### Muscle Activation Detection

A method of detecting muscle activation includes, with a magnetic field sensor, detecting lateral vibration of a magnetic target implanted at a muscle or tendon and estimating a level of muscle activation based on the detected vibration.

A system for detecting muscle activation includes a magnetic field sensor configured to detect lateral vibration of at least one target implanted at a muscle or a tendon, the at least one target comprising a magnetic material. The system further includes a controller configured to estimate a level of muscle activation based on the detected lateral vibration.

### Physiological Parameter Estimation

A method of estimating a physiological parameter of a muscle or tendon, or a muscle-tendon unit, includes, with a magnetic field sensor, detecting vibration of a magnetic target implanted at a muscle or tendon and estimating at least one of a muscle force and tendon force based on the detected vibration.

A system for estimating a physiological parameter of a muscle or tendon, or a muscle-tendon unit, includes a magnetic field sensor configured to detect vibration of at least one target implanted at a muscle or tendon, the at least one target comprising a magnetic material. The system further includes a controller configured to estimate at least one of a muscle force and tendon force based on the detected vibration.

### Portable Goniometry

A method of monitoring biomechanical motion includes disposing at least one target on a subject at a location associated with a joint of the subject and, with a magnetic field sensor array, detecting a change in state of the at least one target relative to the magnetic field sensor array, another target disposed on the subject, or a combination thereof. The method further includes determining a state of the joint based on the detected change in state of the at least one target.

### Improvements to Magnetic Object Tracking

A method for determining one or more of three sensor position parameters and three sensor orientation parameters for each of the sensors in a sensor array includes placing at least one target in at least one known location relative to a sensor array, whereby a signal from the at least one target at the sensors is detected, and recording at least one measurement of the signal at each of the sensors for each placement of the one or more targets. The method further includes estimating one or more parameters from the group consisting of x-position, y-position, z-position, yaw, pitch, and roll, of each of the sensors and estimating any unknown state parameters of the at least one target. A constant value for a magnetic dipole weight state parameter of the at least one target for each of the measurements is provided, and predicted values of the signal at each of the sensors for each of the measurements given the one or more estimated sensor parameters, the estimated target state parameters, and the provided constant magnetic dipole weight state parameter are calculated. A prediction error in the predicted values of the signal with reference to the values of the signals detected at the sensors is computed. A prediction error Jacobian matrix is calculated by analytically computing elements of the prediction error Jacobian matrix with respect to the estimated parameters of the sensors for each measurement and, from the prediction error and the prediction error Jacobian matrix, a state of the parameters of the sensors is determined.

A method of tracking one or more permanent magnets includes detecting a signal from each of the one or more permanent magnets, calculating an analytically-derived Hessian matrix with respect to the detected signals, and determining a state of each of the one or more permanent magnets based on the calculated Hessian matrix.

A system comprises at least two sensor arrays, each sensor array configured to detect a state of at least one magnetic target at a tissue, and at least one position sensor associated with at least one of the at least two sensor arrays and configured to detect a position and orientation of the associated sensor array relative to the other of the at least two sensor arrays.

### Calibration Improvements for Magnetic Field Sensor Arrays

A method of calibrating a magnetic field sensor array that comprises a plurality of magnets includes three-dimensionally rotating a magnetic field sensor array in a uniform magnetic field and recording data from each of the plurality of sensors. The method further includes calculating a non-rotating transformation of the recorded data using an ellipsoid fit of each of the plurality of sensors and calculating a scaling factor of each of the plurality of sensors based on relative dimensions of the ellipsoid fit. A transformed, scaled dataset for each of the plurality of sensors is obtained through application of the calculated non-rotating transformation and calculated scaling factor. The obtained transformed, scaled datasets are rotated to align, thereby providing for a determination of a relative orientation of each of the plurality of sensors to calibrate the magnetic field sensor array.

### Wearable Shielding and Non-Planar Sensing Arrays

A wearable shielding assembly comprises an array of sensors configured to detect a state change of at least one magnetic target implanted at a tissue, a wearable receptacle within which the array of sensors is disposed, and a geometrically-reconfigurable material disposed about or integral with the wearable receptacle. The geometrically-reconfigurable materials provide magnetic shielding to the array of sensors.

A method of assembling a non-planar sensing array, such as for a prosthetic device, includes fabricating a plurality of sensors on a flexible circuit board and affixing the flexible circuit board is to a rigid substrate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic of an example magnetomicrometry and mechanomyography system that includes magnetic targets that passively transmit position information through the tissue to magnetic field sensors located external to the body.

FIG. 2 is a schematic of a wearable robot controlled, at least in part, by a magnetomicrometry system.

FIGS. 3A and 3B are photos of an example magnetic field sensing array and which was used in experimentation. Two 6×8 magnetic field sensor grids were custom designed and held together using a 3D-printed fixture and nylon nuts and bolts (FIG. 3A). The sensor positioning fixture was also used to house a custom adapter board (FIG. 3B) which connected the microcontroller to the sensors via flat flexible cables.

FIG. 4 is a schematic of an experimental set up for comparison of prototype magnetomicrometry devices with fluoromicrometry. A motor was used to apply a mechanical frequency sweep from 0.7 to 7 Hz, with a spring to provide an opposing force. A laptop computer and a magnetic field sensor array mounted external to the turkey's leg were used to track the distance between the magnetic beads (magnetomicrometry), and fluoromicrometry (X-ray stereo videofluoroscopy) was used to obtain a comparison measurement of the distance between the beads.

FIGS. 5A-D are graphs of measured distance over time for first (FIG. 5A), second (FIG. 5B), third (FIG. 5C), and fourth (FIG. 5D) trials performed with a turkey (Turkey A). In each graph, magnetomicrometry (blue, line A) against fluoromicrometry (orange, line B) is plotted, with absolute difference (green, line C), for the frequency sweep performed on the gastrocnemius of Turkey A. All plots correspond to trials from the same leg (right side).

FIGS. 6A-6F are graphs of measured distance over time for first (FIG. 6A), second (FIG. 6B), and third (FIG. 6C) trials performed on the left leg of another turkey (Turkey B), and first (FIG. 6D), second (FIG. 6E), and third (FIG. 6F) trials performed on the right leg of the turkey. In each graph, magnetomicrometry (blue, line A) against fluoromicrometry (orange, line B) is plotted, with absolute difference (green, line C), for the frequency sweep performed on the gastrocnemius of Turkey B.

FIGS. 7A-7F are graphs of measured distance over time for first (FIG. 7A), second (FIG. 7B), and third (FIG. 7C) trials performed on the left leg of yet another turkey (Turkey C), and first (FIG. 7D), second (FIG. 7E), and third (FIG. 7F) trials performed on the right leg of the turkey. In each graph, magnetomicrometry (blue, line A) against fluoromicrometry (orange, line B) is plotted, with absolute difference (green, line C), for the frequency sweep performed on the gastrocnemius of Turkey C.

FIGS. 8A-8F are graphs of measured distance over time for first (FIG. 8A), second (FIG. 8B), and third (FIG. 8C) trials performed on the left leg of yet another turkey (Turkey D), and first (FIG. 8D), second (FIG. 8E), and third (FIG. 8F) trials performed on the right leg of the turkey. In each graph, magnetomicrometry (blue, line A) against fluoromicrometry (orange, line B) is plotted, with absolute difference (green, line C), for the frequency sweep performed on the gastrocnemius of Turkey D.

FIG. 9 is a histogram of differences between the magnetomicrometry and fluoromicrometry gastrocnemius frequency sweep measurements, in micrometers. Different histogram colors correspond to all four trials with Turkey A right leg (green), all three trials with Turkey B left leg and right legs (purple and red, respectively), all three trials with Turkey C left and right legs (blue and orange, respectively), and all three trials with Turkey D left and right legs (cyan and brown, respectively). The mean difference and standard deviation of the difference, in micrometers, is shown to the right of the histogram.

FIG. 10 is a grid of histograms of magnetomicrometry and fluoromicrometry static measurements. Fluoromicrometry (orange) and magnetomicrometry (blue) measurements were taken while magnets were placed at separation distances of approximately 24, 40, 56, and 72 mm in a LEGO block (vertical dashed gray lines in each column), with the sensing array at various heights above the magnets (sensor proximity, shown to the right of the plots.

FIG. 11 is a grid of histograms of magnetomicrometry and fluoromicrometry, static measurements. Fluoromicrometry (orange) and magnetomicrometry (blue) measurements were taken while magnets were placed at separation distances of approximately 24, 40, 56, and 72 mm in a LEGO block (horizontal dashed gray lines in each row), with the sensing array at various heights above the magnets. In contrast with FIG. 10, the vertical axis shows the separation distances, and the sensor proximity is shown at the top of the plots. Magnetomicrometry data is plotted for one second before and one second after the start and end of the fluoromicrometry measurements, respectively. Note that some of the fluoromicrometry measurements also exhibit a warm-up time lasting a small fraction of a second.

FIG. 12 is a plot of histograms of single-board magnetomicrometry static measurements. Magnetomicrometry measurements (blue) were taken while magnets were placed at a separation distances of approximately 24 mm in a LEGO block (indicated by the vertical dashed gray line), with the sensing array at various heights above the magnets (sensor proximity), shown to the right of the plots.

FIG. 13 is a graph of separation distance over time for long-term implant stability of 3mm-diameter magnet pairs against migration in muscle. Pairs of 3mm-diameter magnets were implanted with various separation distances into the gastrocnemius (blue lines, labelled “G”) and iliotibialis cranialis (brown lines, labelled “IC”) muscles of four turkeys. Separation distances were monitored over time via computed tomography scans. Note that there is a cutoff point (around 20 mm for the 3-mm-diameter magnets used) where magnets should not be implanted any closer to one another, to ensure stability against migration.

FIG. 14 is a graph of force between 3-mm-diameter magnets as a function of magnet separation distance. Force between a pair of 3-mm-diameter magnets (shown in blue) at various separation distances, assuming alignment along the line of separation between the magnets. Blue shading represents the forces calculating using the magnetic dipoles strengths plus and minus their respective measurement standard deviations. For reference, the force due to gravity on each magnet is indicated by an orange horizontal line.

FIG. 15 is a schematic of an example magnetic target.

FIG. 16 is a flow diagram of an example method of magnetic-target-based mechanomyography.

FIG. 17 is a diagram illustrating an example control scheme using magnetic-target-based mechanomyography and magnetomicrometry for control of a robotic prosthesis.

FIG. 18 is a schematic of a portable goniometer.

FIG. 19 is a schematic of a magnetic bead insertion device.

FIG. 20 is a schematic of a phantom magnet below a circular sensor array configuration as a visual of a proof performed. A circular sensor array is shown measuring the magnetic field from a north-side-up magnet centered at a height of approximately 0.707 times the sensor circle radius. A north-side-down magnet below the array (outlined in dashed black) creates the same magnetic fields at each of the sensors, and thus is an alternative solution, or “phantom” magnet.

FIG. 21 is a flow diagram illustrating an example method of performing a refined dipole measurement.

FIG. 22 is a schematic of a magnetic field sensor and rotation in a spatially-uniform ambient magnetic field for use in accounting for hard and soft iron effects.

FIGS. 23A-C are plots of calibration data after ellipsoid-to-sphere transformation. Each plot shows the three-dimensional calibration data, after ellipsoid-to-sphere transformation, from a different axis, in particular, as viewed from the positive z-direction (FIG. 23A), positive x-direction (FIG. 23B), and negative y-direction (FIG. 23C). Distinct colors/shading of the dots correspond to the different sensors in the array. Note how the transformation results in data streams which are approximately aligned, and how the data is approximately spherical and centered at the origin.

FIG. 24 is a flow diagram of a calibration process including relative sensor rotation.

FIG. 25 is a graph illustrating measured ambient magnetic field demonstrating the magnetic field effects of a passing train. Labels on the right correspond to the x, y, and z axes from top to bottom. Data was sampled at 300 Hz, so this measurement covers about 40 seconds of magnetic field measurement, with the train passing event lasting about 10-15 seconds.

FIG. 26 is a schematic of an example wearable shielding assembly including geometrically-reconfigurable material.

FIG. 27 is a schematic of another example wearable shielding assembly including geometrically-reconfigurable material.

FIG. 28 is a flow diagram representing common subexpression elimination for Jacobian matrix elements. The color represents the number of floating point operations required to get to each variable, in the perceptionally uniform viridis colormap, incrementing from bright yellow to dark purple. To appreciate the efficiency gained from common subexpression elimination, note, for instance, how x ⁻²ij, y ⁻²ij, z ⁻²ij are each only computed once, as opposed to the 212 total times that they would be computed using Eqns. 11a-r in the Taylor Reference.

FIG. 29 is a flow diagram representing common subexpression elimination for orientation parameters. The color represents the number of floating point operations required to get to each variable, in the perceptually uniform viridis colormap, incrementing from bright yellow to dark purple (though it is noted that sines and cosine generally require significantly more time to implement than, for instance, addition or multiplication). All of the output variables from this flow diagram can be used in the computation of the Jacobian matrix elements, whereas only c₀, c₁ and c₂ are used in the calculation of the magnetic field prediction.

FIG. 30 is a flow diagram representing common subexpression elimination for magnetic field predication calculation. The color represents the number of floating point operations required to get to each variable, in the perceptionally uniform viridis colormap, incrementing from bright yellow to dark purple. Note that c₀, c₁ and c₂ are calculated as shown in FIG. 29.

FIG. 31 is a schematic of a system comprising multiple sensing arrays for which positions, or poses, can be tracked.

FIG. 32 is a flow diagram of an example method of monitoring poses of a multiple sensing array system.

FIG. 33 is a flow diagram of an example method of ranking sensors of a multi-sensor system for a target.

FIG. 34 is a schematic of a non-planar sensing array.

FIG. 35 is a Jacobian matrix constructed with a single magnet measured by N sensors over K timesteps.

FIG. 36 is a sensor position calibration matrix.

### DETAILED DESCRIPTION

A description of example embodiments follows.

Magnetomicrometry is a technology that tracks visually-obscured magnetic beads implanted within or on biological tissue. For example, magnetomicrometry methods and devices described herein can be applied to monitor in-vivo tissue length and speed within freely moving animals and humans.

The methods and devices described herein can be implemented in various technologies, such as for the treatment of limb pathology resulting from disease or traumatic injury and for human augmentation to enhance human physicality beyond normal physiological limits. In the realm of permanent assistance devices, the methods and devices described herein can be implemented in technologies for the preservation of post-amputation function in the residuum for the case of limb amputation, or for the restoration of natural muscle control function in paralyzed or weakened limbs due to age-related degeneration, spinal cord injury, or other neuromuscular pathologies.

Descriptions of systems and methods relating to tracking of magnetic objects can be found in WO2019/074950, “Method for Neuromechanical and Neuroelectromagnetic Mitigation of Limb Pathology;” the entire teachings of which are incorporated herein by reference.

Additional descriptions of systems and methods relating to tracking of multiple targets, such as permanent magnets, can be found in the following publication: Cameron R Taylor, Haley G Abramson, and Hugh M Herr. Low-latency tracking of multiple permanent magnets. IEEE Sensors Journal, 19(23):11458-11468, 2019 (the “Taylor Reference”); the entire teachings of which are incorporated herein by reference. In some aspects, the features described herein present improvements or extensions of the methods and devices described in WO2019/074950 and in the Taylor Reference.

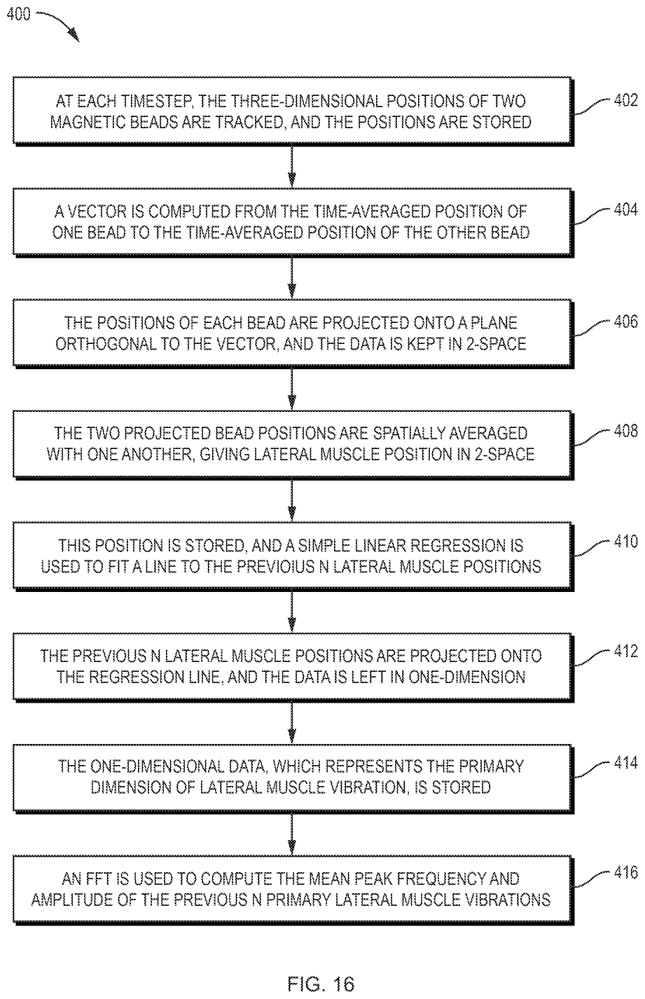

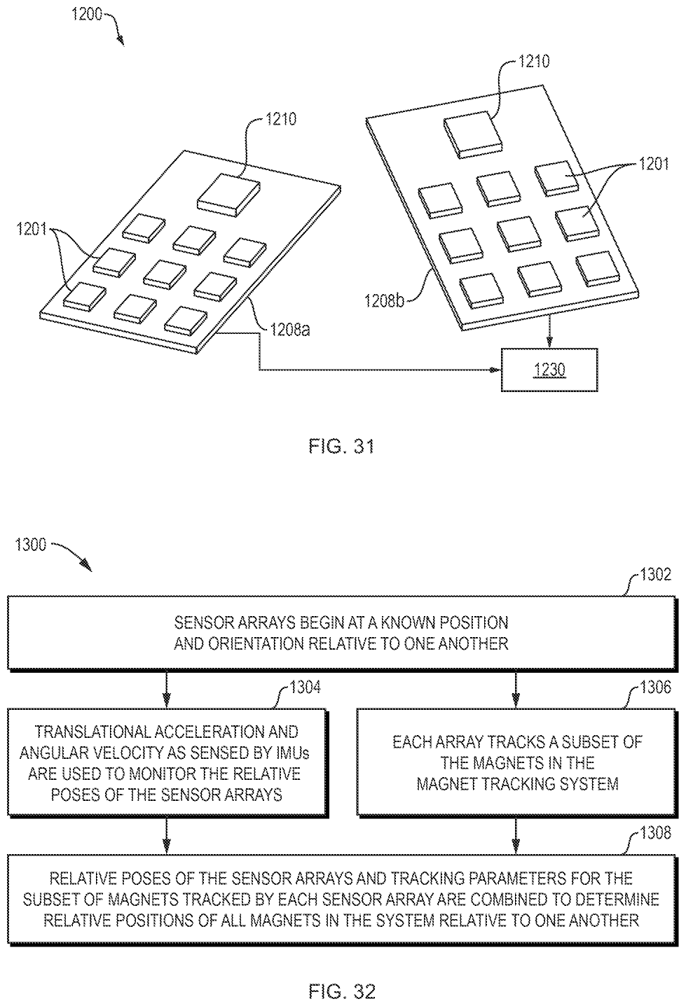

It should be noted that any of the features described herein can be combined with one another and with the features described in the noted publications, in any combination, in a method or system. For example, the monitoring of poses of a multiple sensing array system, as shown in FIG. 32, can be combined with mechanomyographic methods, as shown in FIG. 16. While some examples herein refer to single amputation levels in single extremities, the methods and devices described can be applied to other limbs and amputation levels.

The terms “magnetic bead” and “magnetic target” are used interchangeably herein. Where the term “magnetic bead” is used, it should be understood that the magnetic target need not be of a spherical geometry, and targets of other shapes are possible and included within the meaning of the term “magnetic bead,” including, for example, cube magnets

As defined herein, a “state” of a target, or targets, includes at least one of any of the members of the group consisting of the position, orientation, and strength of the target or targets. Specifically, the “state of the targets relative to each other” includes at least one of any of the members of the group consisting of the relative positions of the targets, the distance between the targets, and the relative orientation between the targets.

There are many methods to track visually-obscured objects. Magnetic-target tracking has the advantages of being low-cost, portable, and safe. However, current magnet tracking technologies are slow, precluding high-speed real-time magnetic-target tracking. This is due to the mathematics of magnet tracking, whereby magnet positions are traditionally determined via numerical optimization, which can suffer from instability and significant delays. Improved methods and systems for tracking one or more magnets with high speed and accuracy are provided. Additionally, validation of such methods is provided through demonstrations of real-time muscle length tracking.

The methods and systems described herein can provide for high-speed, real-time, multiple-magnetic-target tracking. Such methods and systems can use an analytic gradient of a magnetic field prediction error. Magnetic disturbances can be compensated for in real time using a simpler, more portable strategy than currently-published disturbance compensation methods. Validating the method in a physical system against state-of-the-art motion capture, increased maximum bandwidths of 336%, 525%, 635%, and 773% were demonstrated for the simultaneous tracking of 1, 2, 3, and 4 magnets, respectively, with tracking accuracy comparable to state-of-the-art magnet tracking, as further described herein.

Using pairs of implanted magnetic beads to wirelessly track muscle length and speed, a mechanical frequency sweep was applied to an in-vivo turkey gastrocnemius muscle and submillimeter agreement between magnetic-bead-derived real-time muscle length measurements and stereo X-ray videofluoroscopy was found. Longitudinal data using computed tomography was collected, and a minimum magnetic-bead separation distance in muscle of approximately two centimeters was found.

Magnetomicrometry can provide for peripheral nervous system control of wearable robots via real-time tracking of muscle lengths and speeds, as well as for the in-vivo tracking of biological tissues to elucidate biomechanical principles of animal and human movement.

Magnetomicrometry can be used to deliver intuitive, skillful control over bionic prostheses. Magnetomicrometry was developed to meet an immense need in prosthetic control. In the United States alone, there are likely 2 to 3 million persons living with the loss of a limb [25]. However, the commercially-available methods for controlling prosthetic limbs lag behind current robotic prosthesis technologies. On one side of the spectrum are fully non-invasive technologies, such as electromyography, ultrasound, and mechanomyography, all of which reside outside the body, but can have poor, unstable signal quality. On the other side of the spectrum are highly-invasive technologies, such as sonomicrometry, micro-channel arrays, and intrafascicular electrodes. These highly invasive technologies provide improved signal quality, but are expensive to implement and require delicate surgery and careful post-operative care. The field is currently missing a low-footprint, minimally-invasive tissue interface that can accurately monitor muscle actions, as well as muscle activation.

Systems and methods relating to magnetomicrometry, a new technology developed for tracking tissue lengths, speeds and properties, are presented. Magnetomicrometry generally involves implanted magnetic bead(s), which can be used to wirelessly track tissue lengths and tissue states, making it the first ever minimally-invasive real-time muscle tracking technology. Magnetomicrometry systems and methods can also be implemented with simple tracking hardware, making it economical, compact, and portable. With example prototype systems, real-time muscle length tracking with sub-millimeter accuracy and with precision to within a tenth of a millimeter has been demonstrated, as described further herein.

Significant progress has been made recently in the field of prosthetic control and in developing robotic prostheses. State-of-the-art devices, such as, for example, the robotic bebionic® hand (Ottobock, Austin, TX), have many degrees of freedom and are strong, durable tools. However, these devices fall short in their ability to provide full, intuitive, human-like control to the amputee.

The commercially-available method for controlling these advanced prostheses uses electrodes—bare metal conductors placed on the surface of the residual limb. These electrodes measure a signal called muscle activation, and this activation signal is used to control the speed of the prosthesis. Any change in electrode placement results in required recalibration, and even moisture changes throughout the day can degrade the signal. Though our lab, the Biomechatronics Group, has demonstrated significant improvements to this method, called electromyography (EMG), such an approach to prosthesis control has two fundamental limitations.

To accurately map muscle activation to muscle force, both the muscle length and the muscle velocity should be known [8]. A problem arises in that there currently does not exist a technology for measuring these parameters in humans in real time, and it is not possible to accurately determine muscle force, length, or speed from EMG information. Thus, using EMG alone, delivering complete biomimetic control over the prosthesis is difficult. With sensing muscle length and velocity, accurately determining muscle force commands from muscle activation can be provided, or, further, force control can be skipped and direct position control of the prosthesis can be provided. An objective of the provided methods and systems is to provide for full, intuitive, human-like control of a prosthesis by improving the fidelity of force and position control via muscle length and velocity measurements, while eliminating or reducing perceptible control delays.

Magnetomicrometry enables wireless sensing of tissue lengths. For example, a magnetomicrometry approach can use pairs of magnetic beads implanted in tissue, which passively transmit position information to magnetic field sensors external to the body. This technology wirelessly senses muscle lengths and speeds in a low-cost, compact, portable, passive, and safe manner.

Examples of low-latency tracking of multiple permanent magnetics with disturbance compensation are provided in the Taylor Reference [17], the entire teachings of which are incorporated herein.

Section 1 herein describes the results of preclinical work in turkeys with prototype methods and systems for low-latency magnet tracking. Features relating to biocompatibility, accuracy, and long-term implant stability are provided. Section 2 provides for further description of the challenges of magnet tracking, and Section 3 provides for methods for magnetic dipole strength measurement to provide for improved magnet tracking.

Section 4 provides for improvements to calibration of magnetic tracking systems and methods. Section 5 provides for how magnetic field disturbance techniques can be extended to more general disturbances. Section 6 provides for methods for common subexpression elimination towards time delay reduction of magnet tracking methods and tracking optimization. Lastly, Section 7 provides for further general improvements to magnet tracking methods and systems.

### 1. Minimally-Invasive Muscle Tracking Using Permanent Magnets

Low-footprint, minimally invasive tissue interfaces are provided that can accurately monitor muscle actions and that can overcome the various limitations of currently-available technologies. For example, fluoromicrometry, which uses X-rays for high precision tissue length tracking, is wireless but is limited to short bursts due to the ionizing radiation used and requires an entire room and significant processing time [1]. In contrast, sonomicrometry uses physically-tethered, implanted ultrasound crystals to yield high accuracy [14], but it is invasive and not easily miniaturized. And, electromyography, whether via surface electrodes or highly-invasive direct-to-nerve interfaces, only senses muscle activation, which, without muscle length and velocity, cannot be used alone to faithfully observe, understand, or utilize muscle action [8].

Magnetomicrometry is a new technology for tracking tissue lengths and speeds. Implanted magnetic beads are used to wirelessly track tissue lengths, making magnetomicrometry the first-ever minimally-invasive real-time muscle tracking technology. Furthermore, magnetomicrometry systems and methods can use simple tracking hardware, making such systems economical, compact, and portable. With this system, real-time muscle length tracking with sub-millimeter accuracy and with precision to within a tenth of a millimeter has been demonstrated. This new technology, magnetomicrometry, lays the groundwork for full, biomimetic control of prostheses and exoskeletons and an increased understanding of the biomechanical principles of animal and human movement.

Researchers have previously proposed implanting single magnets into muscles in an attempt to monitor movement [16], but the single-magnet-per-muscle approach is limited in various ways. Muscle length can be passively cycled by the motion of a joint, such as the elbow joint when engaged by an aggressive handshake, or actively cycled when flexed, such as when holding a glass of water. These two movements can confound one another if trying to measure either of them via the axial motion of a single magnet, and lateral magnet position can be challenging to use with a prosthesis due to changing pressures from the socket and surrounding tissues. Finally, any motion of the magnetic field sensors or residual limb relative to one another can translate directly into tracking error. These issues can be solved by the use of magnet pairs in each muscle.

In this new tracking strategy, pairs of magnetic beads are wirelessly tracked as described in the Taylor Reference, with lightweight magnetic field sensor arrays mounted to a prosthetic socket. The compact hardware used makes this technology not only portable but also minimally-invasive. Because magnetic fields are not affected by materials such as silicon, carbon fiber, or the human body, the magnetic field can pass undisturbed from the muscles to the sensors as if these other materials were not present.

An example magnetomicrometry system 100 is shown in FIG. 1. The system 100 includes implantable targets 101, 102 (e.g., magnetic beads). As illustrated, the targets 101, 102 are implanted into biological tissue, such as a muscle, e.g., a muscle 105, or a tendon. The targets 101, 102 can passively transmit position information through tissue to magnetic field sensors 106a-e disposed external to the body. The magnetic field sensors 106a-e can be in operative arrangement with a controller 110 to provide measurements for use with magnetomicrometry and/or mechanomyography methods, as described further herein.

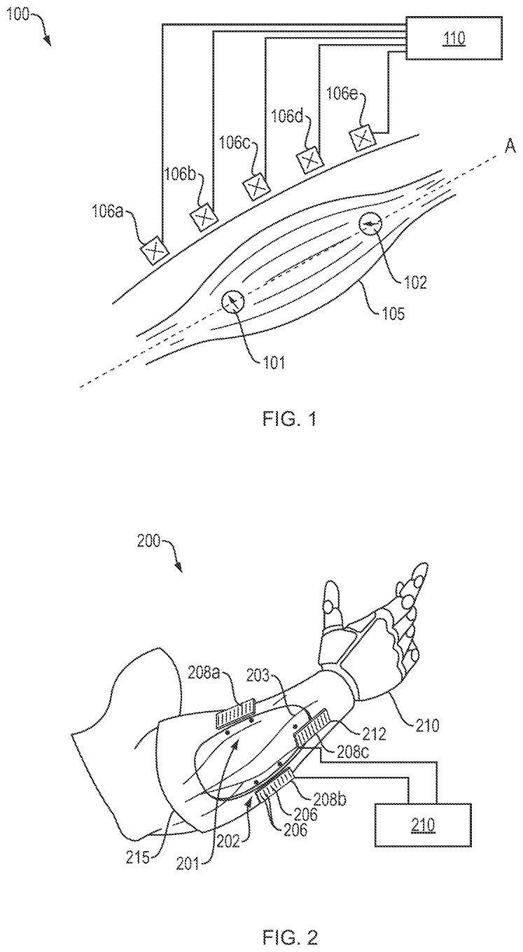

An example of a magnetomicrometry system for prosthetic control is shown in FIG. 2. The system 200 includes pairs of implantable targets 201, 202, and, optionally, an unpaired implantable target 203 (as can be used for mechanomyography, as described later herein). Magnetic field sensing arrays 208a-c are disposed external of the body. The magnetic field sensing arrays can each include one or more magnetic field sensors 206 and, optionally, at least one sensor 212 configured to detect a position and/or an orientation of the array (e.g., an accelerometer, an inertial measurement unit, etc.). Muscle length, velocity, and activation can be measured via detected movement of the implanted targets and used to impart control over a prosthesis 210 (e.g., as illustrated, a robotic hand). The magnetic field sensing arrays 208a-c can be in operative arrangement with a controller 210 (which can be disposed within the prosthesis 210 or an interface 215) to provide measurements for use with magnetomicrometry and/or mechanomyography methods, as described further herein.

An in-vivo turkey model was investigated to address biocompatibility, verify in-vivo tracking accuracy, and ensure long-term resistance of the implants to migration. The study methods and obtained results are further described in Example 1 in the Exemplification section below.

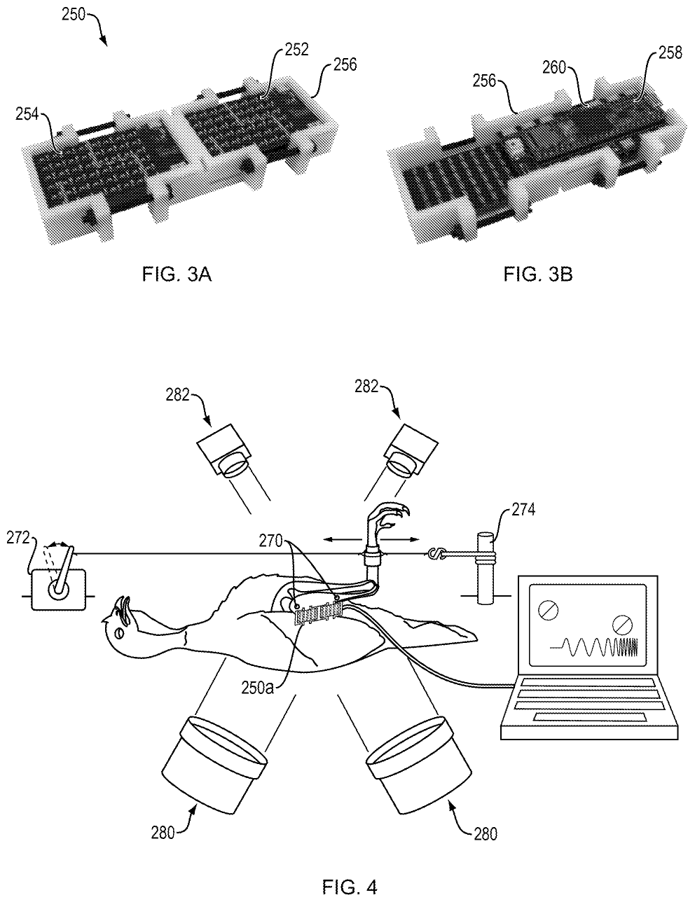

FIGS. 3A and 3B illustrate an example magnetic field sensing array 250, as was used in the experimentation described in Example 1. The magnetic field sensing array 250 includes two magnetic field sensor grids 252, 254, disposed within fixture 256. The sensor positioning fixture was also used to house a custom adapter board 258 which connected the microcontroller 260 to the sensors via flat flexible cables.

### 1.1 MRI-Safe Sensing Components

As described in Example 1, MRI-safe capacitors were used in the sensor arrays of the prototype devices. Traditional magnet tracking compensates for “hard” and “soft” iron effects, where some components that are rigidly affixed relative the magnetic field sensors cause unwanted disturbance by retaining magnetization or by warping the magnetic field seen by the magnetic field sensors. To combat this, the use of circuit components which do not warp or retain the magnetic field as seen by the magnetic field sensors can be used in magnetomicrometry and mechnomyography systems and devices.

For example, MRI-safe sensing components can be used for electrical components on the sensing array board. Specifically, the use of MRI-safe capacitors (e.g. those which contain little to no ferromagnetic or otherwise magnetizable material) can be used in magnetic field sensors. These MRI-safe capacitors can be on the same or opposite side (top or bottom) of the sensing board circuitry and may be shared between sensors as needed.

This use of MRI-safe capacitors is especially useful when proximity between sensors is tight, because a tight sensor layout can cause the capacitors to be positioned especially close to the sensors. If the capacitors distort the field, this increased proximity enlarges this distortion. With MRI-safe capacitors, the field distortion can be eliminated or reduced such that is no longer a factor for consideration in the positioning of the capacitors and sensors.

### 1.2 Biocompatibility

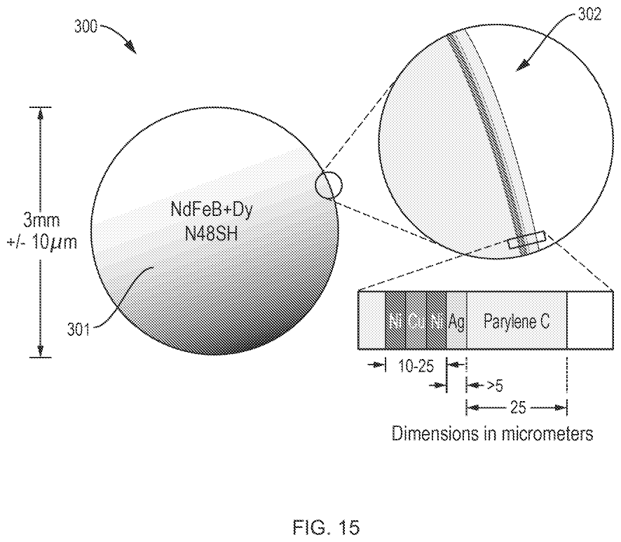

An example of an implantable target for magnetic tracking and providing for biocompatibility is shown in FIG. 15. The target 300 includes a base structure 301 that comprises a magnetic or magnetizable material. For example, the base structure can be formed from a neodymium iron boron+dysprosium base material. The target 301 further includes a shell structure 302 disposed about the base structure that includes layers of nickel, copper, gold, and parylene C.

The shell structure can include layers arranged, from the base structure, in order of nickel, copper, nickel, gold, and parylene C. A gold layer of the shell structure can have a thickness of at least about 5 μm. A paralyene C layer of the shell structure can have a thickness of at least about 25 μm.

An example shell structure is shown in FIG. 15. In the illustrated example, a neodymium iron boron+dysprosium base material is coated in nickel, copper, and then 99.99% pure conventional nickel plating, followed by a layer of gold (e.g., following ASTM B488, Type III, Code A, Class 5 (99.9% Pure Gold, ≤90 HK25 Hardness, at least 5 micrometers thick)), followed by a layer of AdPro adhesion promoter and a coating of Parylene C (e.g., at least 25 micrometers thick).

The gold layer of the shell structure can provide a backup in case of Parylene C failure, and, in addition, can increase the radio-opaqueness of the implant, enhancing the ability to see the implant via alternative technologies (e.g., static x-ray or CT scans). All of these coating steps can be performed with the magnetic beads unmagnetized, with a magnetization step at the very end of the manufacturing process, before or after sterilization.

### 1.3 Muscle Force Measurements: Implanted-Magnetic-Bead-Based Mechanomyography

As discussed above, magnetomicrometry is a tool for measuring muscle length and velocity in real time, and additional information can be needed to discern muscle intents and actions. To do this, magnetomicrometry can be paired with classical strategies, such as surface or needle electromyography, intrafascicular electrodes, or mechanomyography, to determine muscle force. Alternatively, or in addition, muscle force measurements can be obtained from implanted magnetic targets.

Previous work recording vibrations from isolated muscles in saline baths has revealed some underlying principles of mechanomyography, wherein muscle activation can be monitored by acoustic vibrations at the skin's surface using a microphone [3]. These experiments showed that, when activated, each muscle exhibits a lateral vibration (sideways, like a guitar string) on the order of 20-150 Hz. Thus, because the entire muscle vibrates laterally during activation at a frequency higher than that of the muscle flexion itself, the lateral vibration of the same magnetic beads used for muscle length and velocity sensing can be monitored. This lateral vibration frequency can then be used to estimate muscle activation, and, when combined with the length and velocity of the muscle, muscle force can be estimated using a biophysical muscle model (e.g., Hill Muscle Model, [2]). The detection and measurement of lateral vibrations can depend on the precision of magnetomicrometry and the amplitude of the lateral vibrations. The magnetomicrometry system described in Example 1 appears to be capable of monitoring lateral vibrations as low as around 10 to 20 micrometers, which can be sufficient for providing muscle force measurements.

A method of detecting muscle activation includes, with a magnetic field sensor (e.g., sensors 106a-e, or sensor arrays 208a-c), detecting lateral vibration of a target implanted at a muscle or tendon (e.g., targets 101, 102, 201, 202, 203). The method further includes estimating a level of muscle activation based on the detected lateral vibration.

For example, detecting lateral vibration can include detecting movements of the target in a range of about 10 μm to about 20 μm. Alternatively or in addition, detecting lateral vibration can include detecting vibrational movement of the target at frequencies of greater than about 10 Hz. The detection of lateral vibration can be of a single target or multiple targets. For example, detecting lateral vibration can include detecting lateral vibration of two or more targets disposed along an axis (e.g., axis A, FIG. 1). Optionally, an accelerometer can be included to detect vibrational movement of the magnetic field sensor relative to the target. In combination with magnetomicrometry to determine a length and a velocity of the muscle, as described above, a muscle force can be estimated based on the estimated level of muscle activation.

Mechanomyography, or acoustic myography, has been used previously for sensing of muscle activation via vibration sensors or acoustic sensors, but it has never been sensed previously through the tracking of one or more magnetic beads. One or more magnetic beads implanted in muscle (such as magnetic beads used for magnetomicrometry) can be employed to convey mechanomyographic signals as magnetic field information to magnetic field sensors external to the muscle. Such information can be conveyed to a computer via a wired or wireless connection, the computer and sensors being powered by a battery or some other power source. Specifically, mechanomyographic signals can result from muscle vibration along a single lateral dimension during muscle flexion. The lateral dimension is generally perpendicular to the longitudinal axis of the muscle.

For example, one or more magnetic beads can be implanted in a muscle, and, during flexion, these one or more magnetic beads undergo vibration along a single lateral dimension. The intensity and frequency of the one or more magnets' vibrations can be determined using the magnetic field information conveyed from the magnetic field sensors. Because the vibrations occur at a frequency higher than biomechanical movements, the lower frequencies (<10 Hz) can be filtered out and the higher frequencies (>10 Hz) can be used to determine muscle activation. This activation signal from mechanomyography can then be combined with muscle length and velocity signals from, for example, magnetomicrometry, providing a robust estimate of muscle force using a biophysical muscle model (e.g., the Hill Muscle Model) capable of predicting muscle force given inputs of muscle length, velocity and activation.

For example, each of one or more magnetic beads implanted in or on tissue can be monitored for position, and the three components of the position of each magnet can be separately analyzed for frequency content using, for instance, a Fourier or wavelet transform. These frequencies, between different components and between different magnets, can then be combined by, for example, weighted averaging to arrive at a final frequency with which to calculate muscle activation.

As a further example, the positions of multiple beads can be averaged before analyzing the frequency content of the three position components of the signal. For example, the previous N samples (where N is an integer) can be used to fit a simple linear regression in 3D-space using the three position components for each of the magnets or for the averaged position, and the frequency content of the resulting one-dimensional spatial information can then be analyzed.

More than one magnetic bead can be used, and knowledge of the placement of the magnetic beads can be employed to improve sensing capability. For example, with one magnetic bead distal and one magnetic bead proximal within a muscle, a line between the distal and proximal magnetic bead positions (e.g., a time-averaged line, or a regression line including additional beads) can be used to reduce the dimensions along which data analysis occurs by projecting the data from each bead onto a plane orthogonal to the line between the beads. The resulting two dimensions of position data for each bead can then be analyzed for frequency content separately, or can be reduced to one dimension using, for example, a simple linear regression through the previous N data points (where N is some integer), before being analyzed for frequency content. Alternatively, the position data from the multiple magnetic beads can be averaged before or after projection into 2D-space. Regardless of the method, a mean peak frequency can be output, and, optionally, an amplitude at this frequency. This single frequency, as well as its amplitude, if calculated, can then provide for calculation of muscle activation.

An example method 400 of magnetic-target-based mechanomyogrpahy is shown in FIG. 16. The method uses, as an example, a pair of implanted magnetic targets. The method includes, for each timestep, storing the three-dimensional positions of the two, tracked magnetic beads (402). A vector is computed from a time-averaged position of one of the two beads to the time-averaged position of the other of the two beads (404). The positions of each bead are projected onto a plane orthogonal to the vector, and the data is kept in 2D-space (406). The two projected bead positions are spatially averaged with one another, providing for lateral muscle position in 2D-space (408). The position from 408 is stored, and a linear regression is used to fit a line to the previous N lateral muscle positions (410). The previous N lateral muscle positions are projected onto the regression line, and the data is left in one-dimension (412). The one-dimensional data represents the primary dimension of lateral muscle vibration and is stored (414). A Fast Fourier Transform (FFT) is used to compute the mean peak frequency and amplitude of the previous N primary lateral muscle vibrations (416).

One or more accelerometers (and, optionally, an additional sensor such as an inertial measurement unit), can be used to identify any vibrations of the magnetic field sensors relative to the magnetic beads, so as to remove noise from the sensor vibrations from the signal of the lateral vibrations of the one or more magnetic beads.

Magnetic beads can be placed at muscles or tendons to sense lateral muscle vibrations.

### 1.4 Shear Wave Tensiometry Via Magnetic Beads Implanted in Muscles, Tendons, or Surrounding Tissue

Methods are provided for measuring a force exerted by or upon muscle-tendon units. Magnetic targets can be affixed external to or implanted within tendons for force measurement. Tendon strain measured via magnetomicrometry can be employed using a tendon elasticity model to estimate tendon force directly.

A velocity of a shear wave introduced to a tendon can be measured via the velocity of the shear wave propagation through that tendon. Such measurements are typically obtained via ultrasound measurements at two locations within the tendon or via measurement of the

acceleration of the tissue superficial to the tendon using two accelerometers [26]. Magnetic targets can provide for improved implementations of shear wave tensiometry in several regards, using one or more magnetic beads implanted at the tissue in or around the muscle-tendon unit. One benefit of using magnetic beads instead of using ultrasound or a pair of accelerometers is that magnetic beads can both provide high-resolution information in three-dimensions while doing so with a very small time delay. For example, using one or more magnetic beads, a shear wave can be measured in both transverse dimensions of the tendon, even if they are out of phase with one another. In a further example, two or more magnetic beads can be used, with the time delay between the vibrations of the beads being used to determine the velocity of shear waves or compression waves.

A method of estimating a physiological parameter of a muscle or tendon includes, with a magnetic field sensor (e.g., sensors 106a-e, or sensor arrays 208a-c), detecting vibration of a target implanted at a muscle or tendon (e.g., targets 101, 102, 201, 202, 203). The method further includes estimating at least one of muscle force and tendon force based on the detected vibration.

The method can further include applying a perturbance to the target or to a muscle-tendon unit comprising the muscle and the tendon. A timing of the vibration of the target relative to a timing of the perturbance or relative to a timing of a vibration of one or more additional targets implanted at the muscle-tendon unit can be measured. A speed of a shear wave or a compression wave in the muscle-tendon unit or surrounding tissue of the muscle tendon unit can be estimated based on the measured timing. The perturbance can include poking or vibrating the muscle-tendon unit or the surrounding tissue. For example, an applied magnetic field, such as can be supplied by an electromagnet, can actuate a magnetic bead affixed at the muscle-tendon unit or the surrounding tissue to initiate the perturbance. A physiological property of the muscle-tendon unit based on the estimated speed of the shear wave or the compression wave can be determined. The physiological property can be, for example, stiffness of the muscle, the tendon, or the surrounding tissue.

For example, pairs of magnetic beads can be positioned both in the tendon, both in the muscle, one in tendon and one in muscle, or both in tissue surrounding or near the tendon or muscle, including on the surface of the skin external to the body. These waves can be created

using a piezoelectric tapper as in [26], or they can be generated by some other vibration instrument. Alternatively, the vibrations can be caused by movement of the body or by the lateral vibrations of the muscle during activation, and, optionally, this muscle movement can be sensed using electromyography or magnetic-bead-sensed lateral vibrations, as described above. In an example method, a time delay between the source of the compression or shear wave (whether from a tapping device, muscle movement, electromagnet, or some other source) and the measurement of the vibration (whether via a magnet in the muscle, tendon, or surrounding tissue, including on the surface of the skin external to the body) can be used to determine the velocity of the wave. In a further example method, magnetic beads can be used to distinguish between mechanomyographic lateral vibrations, shear waves, and compression waves. For example, because mechanomyographic lateral vibrations are associated with muscle fibers and shear waves are associated with tendon fibers, the differences in physiology can give rise to differences in parameters such as wave velocity or wavelength, allowing the two measurements to be performed using overlapping sets of sensors. An electromagnetic coil can be used to vibrate a magnetic bead to create the shear wave or compression wave.

Another advantage provided by the use of magnetic beads is that the reflection from a transient wave, albeit smaller than an original wave, may be sensed with greater certainty. This allows for an ability to measure wave velocity using a single magnetic bead, or to provide a refined measurement when measuring wave velocity using multiple beads. Or in a further example, this can allow the use of a magnetic bead near the musculotendinous junction to be used to measure delay between the traveling of a tranverse wave from the muscle into the tendon and its reflection back into the muscle after traveling and returning the length of the tendon.

### 1.5 Applications

The use of magnetic targets, as described above, can provide for improvements and new capabilities in biomechatronics, including, for example improving prosthetic control, both in controlling robotic hands, as well as in controlling robotic legs and feet. Such methods and devices can also provide for improved control over orthotic and exoskeletal devices for support, rehabilitation, and augmentation. Additional applications include untethered animal and human biomechanics studies in natural environments, which can elucidate new principles of motion; virtual reality hand and tool tracking, which can benefit from this new tracking strategy due to its ability to provide fast, accurate position information without the need for line-of-sight; and, sensing of muscle lengths and velocities in real time, which can enable closed-loop muscle stimulation control feedback, opening the door to improved strategies for paralysis mitigation and recovery.

### 1.5.1 Control of Robotics Via Magnetic Beads

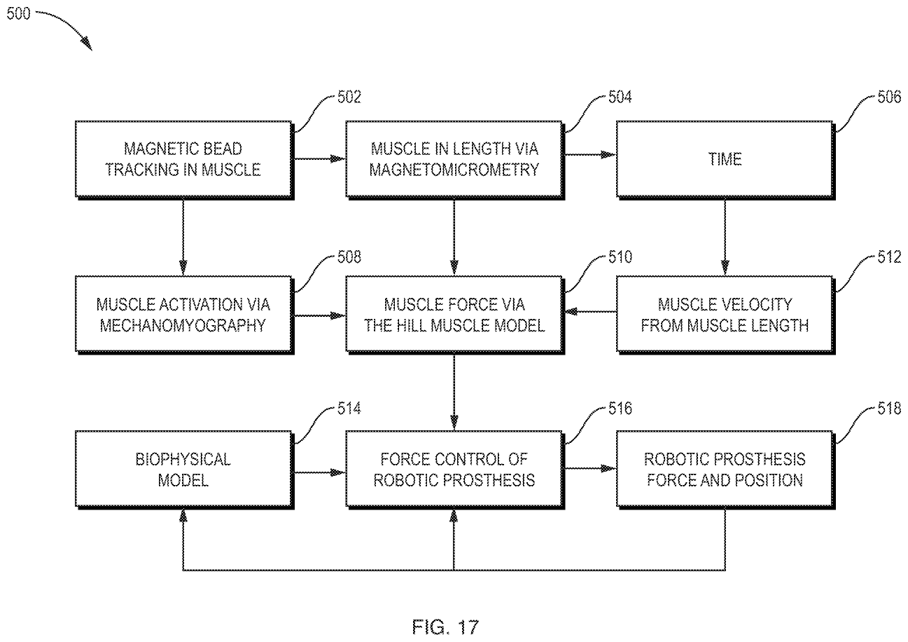

There are various ways that implanted magnetic beads can be used for the control of robotics, such as wearable robotics such as prosthesis, orthoses, and exoskeletons, or other external robotic devices such as humanoids, cars or power tools. An example control diagram is provided showing how magnetomicrometry and mechanomyography can be used in the control of, specifically, a robotic prosthesis (see FIG. 17). However, the same or similar control methodologies can be used for the control of an exoskeleton, orthosis or other such device.

As shown in FIG. 17, tracking of magnetic beads (502), as described above, can provide for muscle length measurements via magnetomicrometry (504) and muscle activation measurements via mechanomyography (508). With the monitoring of time (506), muscle velocity can also be obtained (512). Any or all of the obtained muscle length, muscle velocity, and muscle activation can be provided to a biophysical muscle model (e.g., the Hill Muscle Model) capable of predicting muscle force given inputs of muscle length, velocity and activation (510). The obtained muscle force can then be provided for force control of a robotic prosthetists (516). Force and/or position measurements obtained from the prosthesis can provide for closed-loop control (518) by providing feedback to a biophysical model of the anatomy (514) and to the force control paradigm (516).

Of course, there are many variations with which this control diagram can be used. For example, instead of using force control (516) to control a robotic prosthesis (e.g., prosthesis 210), the muscle length (504) or velocity (512) can be used to control the device using position or velocity control, respectively, in a master-slave configuration, or the force can be used to control the position or the velocity of the device. Alternatively, or in addition, electromyography can provide for mechanomyography in this control scheme. Any of these control methods can be performed with either open-loop or closed-loop control (e.g., feeding back information from the prosthesis as to position, velocity, or force), or using control methodologies, such as Kalman filtering, in the control. Any subset or combination of the inputs, outputs, and methodologies shown in FIG. 17 can be provided for control of a robotic prosthesis.

Furthermore, biological feedback mechanisms such as electrical stimulation, magnetic bead vibration via external coils, or tactile feedback via skin pressure or vibration, can be included in the control of the device. Further still, a measured length, velocity, and force of the muscle can be combined with a biophysical model of the limb including inertia terms to determine biomechanical information, such as joint states and torques, about the user of the device, which can then be fed back into the control algorithm for closed-loop kinetic or kinematic control.

### 1.5.2 Portable Biomechanics Monitoring Using Magnetic Beads—Portable Goniometry

Magnetic targets can be used for motion capture biomechanical studies “in the wild.” Due to the traditional need for an array of cameras, each of which requires line-of-sight to biomechanical markers, motion capture data collections are typically confined to a small lab space. The freedom to have a biomechanical subject (human or animal) move about in a natural setting imparts the ability to study the subject in a more relevant environment and also allows for the biomechanical signals to be used for additional human-computer interfacing. Further, the ability to monitor biomechanical motion capture markers without a need for line of sight allows for the subject to wear comfortable, loose fitting clothing during biomechanical monitoring, encouraging the subject to move in a more natural manner.

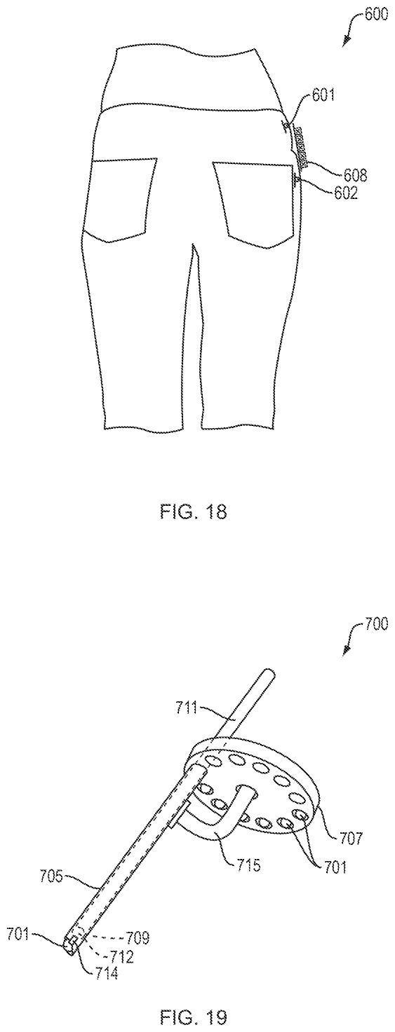

An example of a magnetic bead position sensing system and method that can provide for freedom in biomechanical motion capture monitoring is shown in FIG. 18. As shown in FIG. 18, the system 600 includes magnetic targets 601, 602 disposed at a joint (e.g., the hip) of a subject. A magnetic field sensing array 608 is disposed nearby the targets.

A method of monitoring biomechanical motion includes disposing at least one target (601, 602) on a subject at a location associated with a joint of the subject, and, with a magnetic field sensor array (608), detecting a change in state of the at least one target relative to the magnetic field sensor array, relative to another target disposed on the subject, or a combination thereof. A state of the joint can then be determined based on the detected change in state of the at least one target.

The human body has a diverse array of joints, with differing degrees of freedom and differing ranges of motion, thus requiring an array of different sensing options. Systems and methods involving magnetic target tracking can take many different forms to account for this diversity in ability of movement. It is not sufficient, for instance, to use a protractor to describe the position or the orientation of the shoulder and upper arm, because the shoulder allows the arm to move up and down (abduction/adduction), as well as forward and back (flexion/extension), as well as in rotation.

At least one magnetic bead can be used as a biomechanical marker to track a position or orientation of a joint for biomechanical monitoring. When the magnetic field sensing array is used as a biomechanical marker itself, the one or more beads can be tracked in position and orientation relative to the sensing array and to each other. When the magnetic field sensing array is not itself used as a biomechanical marker, the two or more beads can be tracked relative to one another. The biomechanical markers (e.g., the sensing array, if used as a marker, as well as the one or more magnetic beads) can be affixed at a body segment. For example, the markers can be fixed using an adhesive, such as tape, or the markers can be manufactured as a component of a sticker which affixes to the surface of the skin, or the markers can be embedded in clothing (e.g., tightly worn clothing). To enable the biomechanical monitoring to be performed more accurately or with fewer sensors, the orientations of the magnetic markers can be indicated to ensure optimal placement. For every position or orientation of the markers relative to one another, the position and orientation of the body segments relative to one another can be calculated.

As one example (see FIG. 18), the human hip can be monitored with two degrees of freedom using a magnetic marker placed directly on the surface of the skin over the lateral edge of the ilium (the outside of the hip bone), with the north pole directed anteriorly (directed forward, or toward the front of the body), a magnetic marker placed directly on the surface of the skin laterally to the femur toward the proximal end of the upper leg, with the north pole directed distally (directed down the leg, or toward the knee), and a magnetic field sensing array not used as a biomechanical marker (so that it is free to “float”) can be placed outside of clothing between the two markers to monitor their positions relative to one another. The relative positions and angles of the two magnetic markers can then be used to identify the position of the femur relative to the pelvis.

Methods and systems for magnetomicrometry, as described herein, can provide for tracking of the target(s) and/or sensor array(s). For determination of a joint state, tracking information obtained can be combined with information relating to the anatomical structure of the portion of the body at which the targets are disposed, including, for example, the type of joint and a number of degrees of freedom of the joint.

### 1.6 Magnetic Bead Insertion Device

As discussed in Example 1, in this early feasibility study, a needle and a pair of surgical scissors were used to make an insertion channel for the magnet, and a hollow plastic tube was used to insert the magnet with the aid of a wooden push rod, after which the muscle and skin were sutured closed and film dressing was applied. In clinical practice, there are instances in which the magnets may be inserted during a surgical procedure with the muscle similarly exposed. To generalize this procedure, a device is provided which is capable of additionally inserting the magnetic beads through the skin boundary without the need of a surgical procedure that exposes the muscle. The device can allow for either percutaneous or surgical implantation of one or more magnetic beads.

An insertion device 700, alternatively referred to as an injector, is shown in FIG. 19. The device includes a cannula 705 and a cartridge 707 configured to position an implantable target 701 at the cannula. A pushrod 711 is received in the cannula and configured to push the implantable target 701 from the cartridge through the cannula for delivery to a tissue. The cannula and the pushrod comprise a nonmagnetic material. A distal end 709 of the cannula can be of a complementary geometry to the implantable target to prevent damage to a shell structure of the target during delivery. For example, where the target is a spherical magnetic bead, the distal end of the cannula can be of a curved geometry. A distal surface 712 of the pushrod can include a spring structure 714 that can prevent damage to the shell structure of the target (e.g., an elastomeric material, a coil spring, etc.). The device can further include a mount 715 coupling the cartridge 707 to the cannula. The cartridge can be configured to retain a plurality of targets and can be rotatable about the mount to position each of the plurality of targets at the cannula.

An insertion kit or an insertion system can include a plurality of implantable targets (e.g., magnetic beads 300) and an insertion device (e.g., device 700). The implantable targets can be deliverable by the insertion device.

As described above in the discussion of fluoromicrometry, nonmagnetic beads have long been used for tracking tissue positions using X-rays, a technique called radiosteriometric analysis (RSA). To perform RSA, the nonmagnetic beads (typically made of tantalum) are injected, often percutaneously, into the tissue to be tracked. This injection process is performed using devices such as the tantalum bead inserter manufactured and sold by RSA Biomedical and Halifax Biomedical.

Magnetic bead tracking differs in that the injected beads are magnetized, and that they are often of larger diameter. Devices, such as injector 700, can inject permanently magnetized implantable components, such as spherical, cylindrical, or cube magnets (or any other geometry of magnet), into human or animal tissue via a nonmagnetic needle (e.g., barrel, cannula, or rigid tube) and using a nonmagnetic pushrod. The needle and the pushrod can be of a same or different material, composed of rigid nonmagnetic materials, such as titanium, copper, nonmagnetic stainless steel, gold, wood, silver, brass, glass, aluminum, zinc, marble, bronze, or a polymer (such as a plastic). The term “nonmagnetic” is defined herein to mean any material that is not temporarily or permanently magnetizable, such that the material does not electromagnetically interact with a permanently magnetized object when the material and the object are static relative to one another and no electric current is introduced into the system. The pushrod or needle can be coated in a biocompatible material to increase the biocompatibility of the surface and to decrease the stiffness of the surface, to decrease the possibility of the biocompatible coating of the inserted component being compromised (for instance, with a coating of gold or parylene).

One or more implantable magnetic components can be inserted into a nonmagnetic cartridge, such as in the revolver or magazine of a firearm, wherein the cartridge is inserted into the device for the injection of the one or more magnetic implants. The cartridge can have magnetized or ferromagnetic components to guide the magnetic flux, to help preserve the magnetization of the permanently magnetized components over time (days to decades), or to hold the beads in a particular location or orientation before being dislodged by the pushrod. The pushrod can include a spring in order to minimize the possibility of excessive force being placed on the bead. The pushrod can have a smooth tip that matches the geometry of the implant being injected into the body, or that is flat, beveled, or domelike. The above instances can be adapted to accommodate magnetic implants of various sizes, for instance spherical magnetic beads of 1 mm, 2 mm, 3 mm, 4 mm, or 5 mm diameter, or cylindrical or cube magnets in a similar range of sizes.

As an example of several of these instances being combined, FIG. 19 shows a plastic pushrod with a curved tip used to dislodge a spherical magnetic bead from a cartridge of multiple beads and guide it down a nonmagnetic stainless-steel cannula into a muscle, though an insertion path that has been created using the sharp end of the cannula. In FIG. 19, the cartridge is mounted so that it can revolve with respect to the cannula, and the pushrod is not rigidly affixed to the other components of the device.

The magnetic bead insertion device can be used in combination with an imaging device, such as ultrasound, an augmented reality device combined with previously-collected Mill data, or a magnetic bead tracking system, to guide the insertion of the magnetic beads.

### 2. Multi-Valued Inverse Existence Proof for N Sensors

### 2.1 Proof of Non-Guarantee of Single-Valued Inverse

It is shown that the inverse of the magnetic dipole field equations is not guaranteed to be single-valued. To do this, a demonstration is provided of the existence of at least one geometry, for any number of sensors and any magnet strength, where the magnet can cause identical magnetic fields at all of the sensors from multiple, distinct locations.

Reference is made herein to the Taylor Reference [17], the entire teachings of which are incorporated herein by reference.

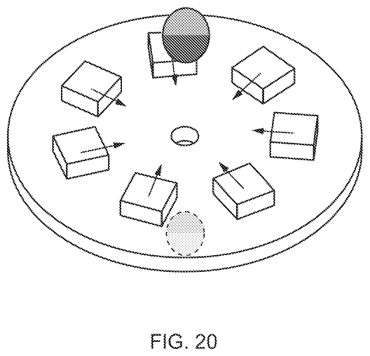

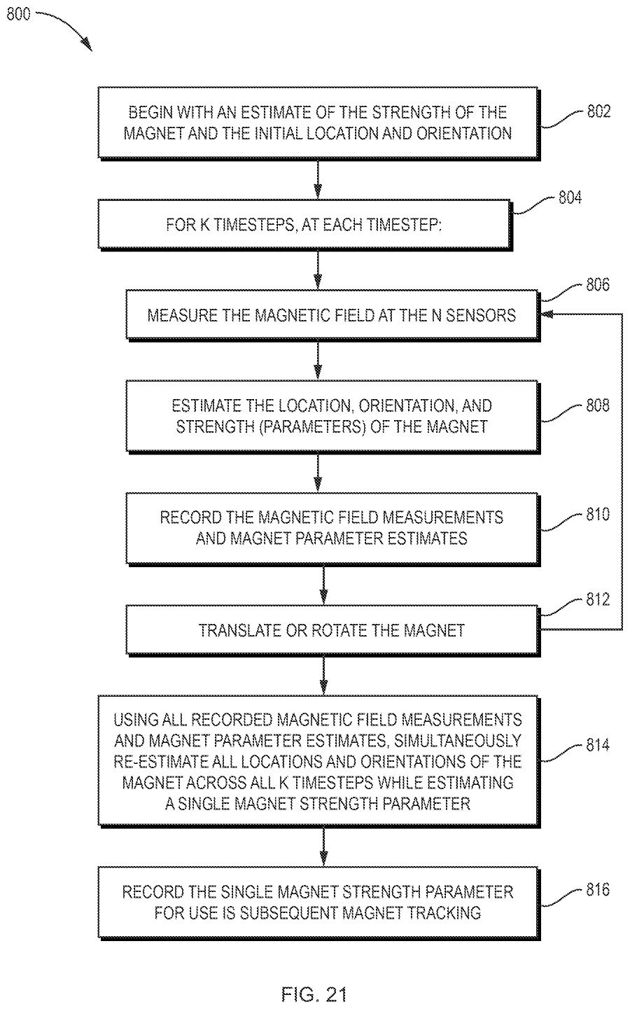

Consider N sensors on a circle, not necessarily evenly spaced, and a magnet oriented normal to the circle, positioned on a line normal to and intersecting the center of the circle, as illustrated in FIG. 20.

To investigate this situation, we first convert equations (8a) and (8c) of the Taylor Reference into cylindrical coordinates, and with y⁻=0 and θ=φ=0, we have

$${B}_{ρ} = \overline{m} ( \frac{3 \overline{ρ} \overline{z}}{{( {\overline{ρ}}^{2} + {\overline{z}}^{2} )}^{5 / 2}} ) \qquad ( 2.1 a )$$

$${B}_{z} = \overline{m} ( \frac{3 {\overline{z}}^{2}}{{( {\overline{ρ}}^{2} + {\overline{z}}^{2} )}^{5 / 2}} - \frac{1}{{( {\overline{ρ}}^{2} + {\overline{z}}^{2} )}^{3 / 2}} ) , \qquad ( 2.1 b )$$

where ρ⁻ is a vector giving any sensor's radial position relative to the magnet, and B_{ρ} is the radially-directed magnetic field sensed at all of the sensors. For simplicity, we consider the case where the magnetic field from the magnet is fully in the plane of the sensors at each of the sensors (i.e., B_{z}=0). We then have

$$0 = \overline{m} ( \frac{3 {\overline{z}}^{2}}{{( {\overline{ρ}}^{2} + {\overline{z}}^{2} )}^{5 / 2}} - \frac{1}{{( {\overline{ρ}}^{2} + {\overline{z}}^{2} )}^{3 / 2}} ) \qquad ( 2.2 )$$

$$= \overline{m} ( \frac{3 {\overline{z}}^{2} - ( {\overline{ρ}}^{2} + {\overline{z}}^{2} )}{{( {\overline{ρ}}^{2} + {\overline{z}}^{2} )}^{5 / 2}} ) \qquad ( 2.3 )$$

$$= \overline{m} ( \frac{2 {\overline{z}}^{2} - {\overline{ρ}}^{2}}{{( {\overline{ρ}}^{2} + {\overline{z}}^{2} )}^{5 / 2}} ) \qquad ( 2.4 )$$

$$⇒ 0 = 2 {\overline{z}}^{2} - {\overline{ρ}}^{2} \qquad ( 2.5 )$$

$$\overline{z} = \pm \frac{1}{\sqrt{2}} \overline{ρ} . \qquad ( 2.6 )$$

Thus, when the magnet is positioned above or below the circle of sensors at a height or depth of approximately 0.707 times the radius of the circle of sensors, the z-directed magnetic field seen by the sensors is zero. Because the magnet is oriented with the north pole facing up, if the magnet is positioned above the array, the magnetic fields are directed radially inwards, but if the magnet is positioned below the array, the magnetic fields are directed radially outwards with the same magnitude. However, reversing the polarity of the magnet reverses the radial direction of the magnetic field without changing the magnitude. For example, a magnet facing north-pole-up at a height above the sensors of 0.707 times the sensor circle radius will produce the same magnetic fields at the sensors as a magnet facing north-pole-down at a depth of 0.707 times the radius under the sensors (see FIG. 20).

Described mathematically, this is shown simply as

$${B}_{ρ} = \overline{m} ( \frac{3 \overline{ρ} \overline{z}}{{( {\overline{ρ}}^{2} + {\overline{z}}^{2} )}^{5 / 2}} ) \qquad ( 2.7 )$$

$$= \overline{m} ( \frac{3 \overline{ρ} ( \frac{1}{\sqrt{2}} \overline{ρ} )}{{( {\overline{ρ}}^{2} + {( \frac{1}{\sqrt{2}} \overline{ρ} )}^{2} )}^{5 / 2}} ) \qquad ( 2.8 )$$

$$= ( - \overline{m} ) ( \frac{3 \overline{ρ} ( - \frac{1}{\sqrt{2}} \overline{ρ} )}{{( {\overline{ρ}}^{2} + {( - \frac{1}{\sqrt{2}} \overline{ρ} )}^{2} )}^{5 / 2}} ) \qquad ( 2.9 )$$

$$= {\overline{m}}^{′} ( \frac{3 \overline{ρ} {\overline{z}}^{′}}{{( {\overline{ρ}}^{2} + {\overline{z}}^{′ 2} )}^{5 / 2}} ) \qquad ( 2.1 )$$

where m bar prime and z bar prime represent, respectively, the magnetic dipole weight of the magnet and the vertical position of the sensors relative to the magnet.

Having shown that, for this particular geometry, at least one multi-valued inverse exists for any strength magnet regardless of the number of sensors used to track the magnet, it is thus concluded that the inverse is not guaranteed to be single-valued for all geometries, regardless of the number of sensors used.

Additional configurations in which multiple, distinct magnet positions yield the same magnetic field at multiple sensor positions are possible. For instance, when tracking magnets with six degrees of freedom in the circular sensor configuration discussed above, for each different magnet height, a phantom magnet of differing magnetic dipole weight with a depth that differs from the magnet height can appear. As a further example, any locations relative to the magnet where the magnetic field vectors are both colinear and either parallel or anti-parallel can create a rotational degree of freedom about a pair of sensors when the colinear parallel or anti-parallel magnetic fields correspond with the sensor locations.

Now, perhaps it can be proved that a single-valued inverse is guaranteed when more than two sensors are used that are not at coplanar locations on a circle. However, the existence proof above is still relevant for these particular cases because it reveals locations of potential local minima and suggests how sensor geometries can be designed to avoid these minima. It also suggests the difficulty in finding an analytic inverse to the magnetic dipole field equation, as the analytic inverse would need to take into account sensor geometry and the possible presence of multiple solutions.

The use of the spatial magnetic field gradient has been shown to allow inversion from magnetic dipole field measurements, giving dipole locations via matrix inversion [23, 10, 22], but these direct-inversion methods require calculating the spatial gradient of the field, which, with presently-available magnetic field sensors, introduces non-negligible numerical error. More importantly, the direct-inversion techniques are currently limited to just a single magnet tracked via a single estimated magnetic field and gradient, calling for further refinement via optimization methods. Extending these direct-inversion techniques to the tracking of multiple magnets using many magnetic field sensors can improve the accuracy of direct-inversion using the spatial gradient and perhaps providing a high-speed, high-accuracy global search strategy for many magnets at once. Modifications to compensate for magnetic disturbance fields can be provided.

### 3. Measurement of Dipoles

The following section presents improvements to the methods and systems relating to tracking of magnetic objects found in WO2019/074950, “Method for Neuromechanical and Neuroelectromagnetic Mitigation of Limb Pathology” and in the Taylor Reference, the entire teachings of which are incorporated herein by reference.

A method for determining one or more of three sensor position parameters and three sensor orientation parameters for each of the sensors in a sensor array includes placing at least one target in at least one known location relative to a sensor array, whereby a signal from the at least one target at the sensors is detected, and recording at least one measurement of the signal at each of the sensors for each placement of the one or more targets. The method further includes estimating one or more parameters from the group consisting of x-position, y-position, z-position, yaw, pitch, and roll, of each of the sensors and estimating any unknown state parameters of the at least one target. A constant value for a magnetic dipole weight state parameter of the at least one target for each of the measurements is provided, and predicted values of the signal at each of the sensors for each of the measurements given the one or more estimated sensor parameters, the estimated target state parameters, and the provided constant magnetic dipole weight state parameter are calculated. A prediction error in the predicted values of the signal with reference to the values of the signals detected at the sensors is computed. A prediction error Jacobian matrix by analytically computing elements of the prediction error Jacobian matrix with respect to the estimated parameters of the sensors for each measurement is calculated and, from the prediction error and the prediction error Jacobian matrix, a state of the parameters of the sensors is determined.

### 3.1 Strength Initialization

In the Taylor Reference, Eqn. 7 was used to calculate an initial estimate of the magnetic dipole weight, followed by a short (˜30 s) six-degree-of-freedom tracking session in which the magnetic dipole weight was continuously tracked and recorded. The median of the recorded magnetic dipole weights for each magnet was then used as the known magnetic dipole weight value for that magnet in five-degree-of-freedom tracking. For maximum stability, accuracy, and bandwidth, each magnet's strength should be measured before tracking (as was performed in this work) whenever an application permits doing so.

It was assumed in the Taylor Reference that the residual flux density (the remanence), Bᵣⱼ, of a magnet is readily accessible information. This parameter is not, however, always reported by magnet vendors. Strategies are shared for using either the “N-Rating” or the surface field of a magnet to determine an initial estimate of the magnet's residual flux density.

In practice, in this work it was found to be tolerable for an initial estimate of the dipole strength to be incorrect by several orders of magnitude when starting six-degree-of-freedom tracking (i.e., when measuring the magnet's strength). However, in multiple-magnet six-degree-of-freedom tracking (e.g., when using a sensor array to measure the strength of two magnets at once), a more accurate initial estimate can improve stability at the start of tracking. Additionally, the formulas below aid in performing simulations to determine which factory-specified magnets will work best for a given application.

For optimal accuracy when sensor repeatability is in doubt, the magnetic dipole weight can be measured using the same sensors and calibration that can be used in five-degree-of-freedom tracking, since the strength that is measured by a sensor array is measured relative to the sensitivity of the given sensors.

Although the methods below are derived for spherical magnets, these same strategies can be used to determine an approximation for the strength of a magnet of similar geometry, such as a cube or a cylinder, for far-field tracking.

3.1.1 Strength from N-Rating

If the residual flux density is known, an initial guess for the magnetic dipole weight can be calculated via Eqn. 7 in the Taylor Reference. If either the N-rating or the magnetization of the magnet are known, these can be used to estimate the residual flux density. (If the magnetization Mⱼ is known

$$( where {M}_{j} = \frac{{m}_{j}}{{V}_{j}} ) ,$$

the residual flux density can be calculated from it as Bᵣⱼ=μ₀Mⱼ.) Using tables from several prominent magnet vendors [6, 7, 19], the rule of thumb can be empirically-derived, where Nⱼ is the N-rating of the jth magnet.

$${B}_{rj} = \frac{\frac{{N}_{j}}{6} + 6}{10} \qquad ( 3.1 )$$

However, if the manufacturer is known and the manufacturer has a table reporting the residual flux density, the manufacturer's residual flux density specifications can be used instead. The magnet specifications as reported by a manufacturer may not line up with the dipole magnitude as seen by the sensors, due to errors in the magnet specifications, the sensor specifications, or in the above empirically-derived formula.

3.1.2 Strength from Surface Field

Alternatively, an estimate of the strength of the magnet can be determined from the surface field of the magnet measured at its north or south pole by a gaussmeter (also known as a fluxmeter or teslameter, a gaussmeter is a magnetometer with a very high full-scale range) or as reported by the factory specifications of a given magnet. The residual flux density of a spherical magnet is not the same as its field at the north pole. Repeating Eqn. 8c of the Taylor Reference here for convenience, the z-directed field is given by:

$${B}_{iz} = {G}_{z} + {\sum }_{{j}^{′} = 1}^{{j}^{′} = M} {\overline{m}}_{{j}^{′}} ( \frac{3 {\overline{z}}_{{ij}^{′}} ( sin {θ}_{{j}^{′}} cos {ϕ}_{{j}^{′}} {\overline{x}}_{{ij}^{′}} + sin {θ}_{{j}^{′}} sin {ϕ}_{{j}^{′}} {\overline{y}}_{{ij}^{′}} + cos {θ}_{{j}^{′}} {\overline{z}}_{{ij}^{′}} )}{{( {\overline{x}}_{{ij}^{′}}^{2} + {\overline{y}}_{{ij}^{′}}^{2} + {\overline{z}}_{{ij}^{′}}^{2} )}^{5 / 2}} -$$

$$\frac{cos {θ}_{{j}^{′}}}{{( {\overline{x}}_{{ij}^{′}}^{2} + {\overline{y}}_{{ij}^{′}}^{2} + {\overline{z}}_{{ij}^{′}}^{2} )}^{3 / 2}} )$$

The surface field at the north pole of the jth spherical magnet is calculated by setting θⱼ=⁻xᵢⱼ=⁻yᵢⱼ=G_{z}=0 and z⁻ᵢⱼ=Rⱼ in this equation, giving

$$\begin{matrix}{B}_{sj} = {\overline{m}}_{j} ( \frac{3 {\overline{z}}_{ij} ( sin {θ}_{j} cos {ϕ}_{j} {\overline{x}}_{ij} + sin {θ}_{j} sin {ϕ}_{j} {\overline{y}}_{ij} + cos {θ}_{j} {\overline{z}}_{ij} )}{{( {\overline{x}}_{ij}^{2} + {\overline{y}}_{ij}^{2} + {\overline{z}}_{ij}^{2} )}^{5 / 2}} - \\ \frac{cos {θ}_{j}}{{( {\overline{x}}_{ij}^{2} + {\overline{y}}_{ij}^{2} + {\overline{z}}_{ij}^{2} )}^{3 / 2}} ) \\ = {\overline{m}}_{j} ( \frac{3 {R}_{j} ( {R}_{j} )}{{R}_{j}^{5}} - \frac{1}{{R}_{j}^{3}} ) \\ = {\overline{m}}_{j} \frac{2}{{R}_{j}^{3}}\end{matrix} \qquad ( 3.2 )$$

Then, comparing (3.2) with Eqn. 7 of the Taylor Reference gives:

Bᵣⱼ= 3/2Bᵣⱼ (3.3)

### 3.1.3 Other Methods

There are other methods to determine the strength of a magnet (for instance, the time integral of a coil rotated about the magnet can be used to determine the magnet strength). Moreover, if the magnet is non-spherical, the above methods can still be used to determine an acceptable initialization. The estimation does not have to be precise in order to begin six-degree-of-freedom tracking. For a single magnet, the initial estimate can be incorrect by several orders of magnitude and still be tracked. However, for multiple magnets, it becomes more necessary, for stability, to have better initial estimates.

### 3.2 Non-Negative Strength Constraint in Six-Degree-of-Freedom Tracking

On a practical note, it was found in this work that the tracking algorithm was more stable if negative magnetic dipole strengths were reversed in sign and orientation during six-degree-of-freedom tracking, especially when tracking multiple magnets at once. In other words, it was found that tracking stability was increased by multiplying m⁻ⱼ by −1 and subtracting π from θⱼ whenever the tracking algorithm determined that m⁻ⱼ<0. This was found to be particularly important in the stability of multi-magnet measurement.

### 3.3 Refinement to Dipole Measurement

It is possible to refine the magnetic dipole weight measurement beyond merely using the median of the recorded magnetic dipole weights during six-degree-of-freedom tracking. The measurement process can first be performed as described above and in view of the Taylor Reference, but, in addition to recording the magnetic dipole weight, the tracking data (e.g., position and orientation of the magnets) can be recorded as well. The median of the magnetic dipole weight, along with the five-degree-of-freedom data across all (or a representative subset) of the tracking timesteps can then be used as an initial estimate for a new optimization. This new optimization can be performed similarly to current tracking methods, except that instead of the error corresponding to a single timestep over all sensors, the optimization takes into account the error across all timesteps over all sensors. In other words, both the path and the magnetic dipole weight can be optimized, and the output can be a refined path with a single magnetic dipole weight corresponding to each magnet.

A description of example optimization is provided. As before, the magnetic field prediction error is used for the optimization cost function. At the ith sensor and kth timestep, the magnetic field prediction error, Eⱼₖ, is the difference between the measured magnetic field B^{\tilde{ }}ⱼₖ and the predicted magnetic field Bⱼₖ,

Eⱼₖ=Bⱼₖ−\tilde{B}ⱼₖ, (3.4)

For each timestep (the kth timestep), the derivatives of the magnetic field prediction errors with respect to each of the full-path magnetic dipole weight estimates are calculated and placed in a separate submatrix

$${M}_{k} = [ \begin{matrix}\frac{\partial }{\partial {\overline{m}}_{1}} {B}_{1 kx} & & \frac{\partial }{\partial {\overline{m}}_{M}} {B}_{1 kx} \\ \frac{\partial }{\partial {\overline{m}}_{1}} {B}_{1 ky} & … & \frac{\partial }{\partial {\overline{m}}_{M}} {B}_{1 ky} \\ \frac{\partial }{\partial {\overline{m}}_{1}} {B}_{1 kz} & & \frac{\partial }{\partial {\overline{m}}_{M}} {B}_{1 kz} \\ \frac{\partial }{\partial {\overline{m}}_{1}} {B}_{2 kx} & & \frac{\partial }{\partial {\overline{m}}_{M}} {B}_{2 kx} \\ \frac{\partial }{\partial {\overline{m}}_{1}} {B}_{2 ky} & … & \frac{\partial }{\partial {\overline{m}}_{M}} {B}_{2 ky} \\ \frac{\partial }{\partial {\overline{m}}_{1}} {B}_{2 kz} & & \frac{\partial }{\partial {\overline{m}}_{M}} {B}_{2 kz} \\ ⋮ & ⋱ & ⋮ \\ \frac{\partial }{\partial {\overline{m}}_{1}} {B}_{Nkx} & & \frac{\partial }{\partial {\overline{m}}_{M}} {B}_{Nkx} \\ \frac{\partial }{\partial {\overline{m}}_{1}} {B}_{Nky} & … & \frac{\partial }{\partial {\overline{m}}_{M}} {B}_{Nky} \\ \frac{\partial }{\partial {\overline{m}}_{1}} {B}_{Nkz} & & \frac{\partial }{\partial {\overline{m}}_{M}} {B}_{Nkz}\end{matrix} ] . \qquad ( 3.5 )$$

Defining Jₖ to be the Jacobian matrix corresponding to the kth measurement, where the elements corresponding to the full-path magnetic dipole weight have been removed and placed in Mₖ, the full-path dipole measurement Jacobian matrix is then constructed as

$$\overline{J} = [ \begin{matrix}{J}_{1} & 0 & … & 0 & {M}_{1} \\ 0 & {J}_{2} & … & 0 & {M}_{2} \\ ⋮ & ⋮ & ⋱ & ⋮ & ⋮ \\ 0 & 0 & … & {J}_{K} & {M}_{K}\end{matrix} ] . \qquad ( 3.6 )$$

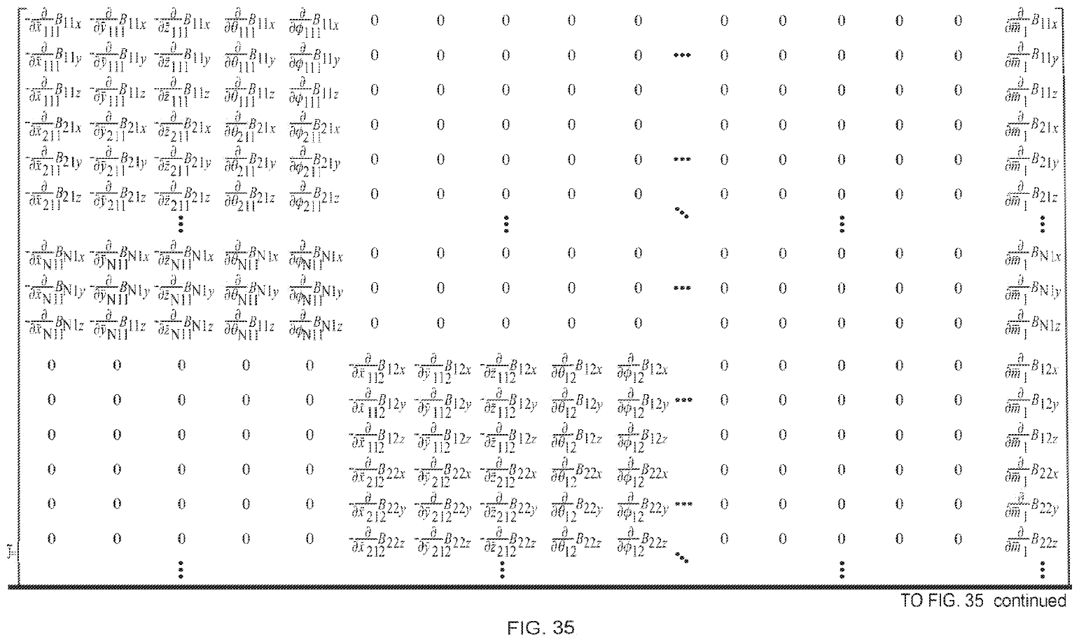

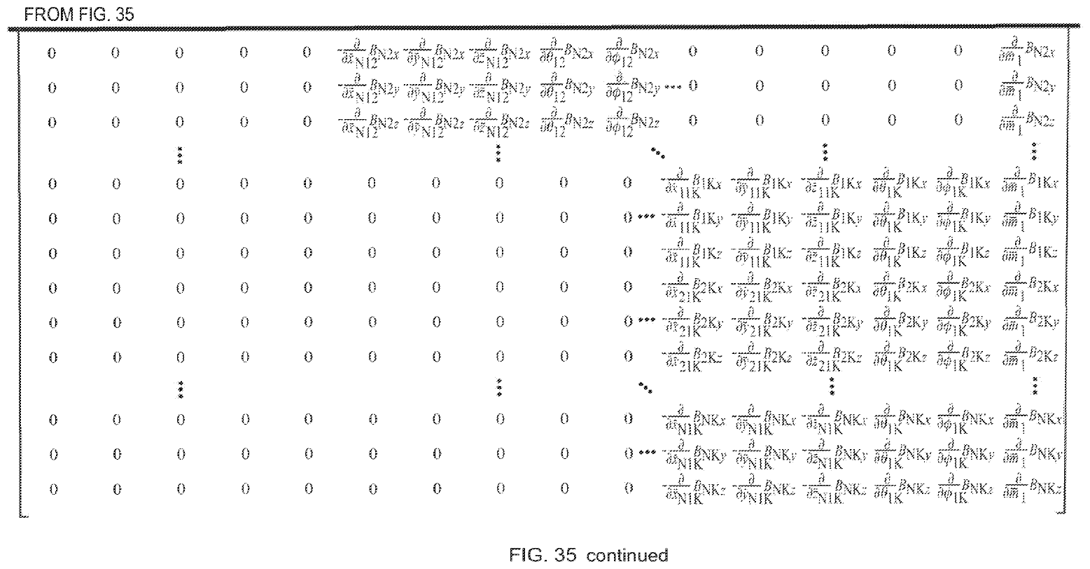

For example, with a single magnet measured by N sensors over K timesteps, this is constructed as shown in FIG. 35, where x⁻ᵢⱼₖ, y⁻ᵢⱼₖ, and z⁻ᵢⱼₖ are the distances in x, y, and z to the ith sensor from the jth magnet at the kth timestep, and θⱼₖ and φⱼₖ are the orientation from and around vertical of the jth magnet at the kth timestep.

Note that, as with the magnetic dipole weight in the example above, any other tracking parameter can be considered constant (with either an unknown or a previously known value) across some or all measurements. For instance, the position can be fixed while the orientation is allowed to change (e.g., the magnet being rotated while positioned in an indentation), the orientation can be fixed while the position is allowed to change (e.g., a magnet fixed in a block that is moved across a surface), and disturbance field of any given complexity can be accounted for and considered varying or constant across the measurements.

FIG. 21 is a flowchart illustrating an example method of performing a refined dipole measurement. The method 800 includes beginning with an estimate of the strength of the magnet and its initial location and orientation (802). For K timesteps, at each timestep (804): the magnetic field at the N sensors is measured (806), the parameters of the magnet (e.g., location, orientation, and strength) are estimated (808), the magnetic field measurements and magnet parameter estimates are recorded (810), and the magnet is translated or rotated (812). Using all or several recorded magnetic field measurements and magnet parameter estimates, the locations and orientations of the magnet across all K timesteps are re-estimated while estimating a single magnet strength parameter (e.g., a constant value) (814). The single magnet strength parameter is recorded for use in subsequent magnet tracking (816).

3.4 Minimum Distance of a Magnet from any Magnetometer

It can be prudent in considering the measurement and tracking of a magnet to also consider the full-scale range of the sensors that are used to measure and track the magnet. In this section, strategies are provided for making a conservative estimate of the minimum distance that is to be maintained between a single permanent magnet and any magnetic field sensor while not exceeding the full-scale range of the sensor.

To calculate a conservative estimate, the equation for magnetic field is reduced to a function of distance from the magnet along the axis that has the greatest magnetic field. The scenario is then considered in which this axis of the magnet is aligned with one of the three axes of the sensor. The axis of a magnet along which the magnetic field is greatest at a given radial distance is the z-axis of the magnet, so Eqn. 8c of the Taylor Reference is used, with M=N=1 and x⁻=⁻y=θ=G_{z}=0 at a distance of z⁻=dₘᵢₙ:

$$\begin{matrix}{G}_{max} = {G}_{z} + {\sum }_{{j}^{′} = 1}^{{j}^{′} = M} {\overline{m}}_{{j}^{′}} \\ ( \frac{3 {\overline{z}}_{{ij}^{′}} ( sin {θ}_{{j}^{′}} cos {ϕ}_{{j}^{′}} {\overline{x}}_{{ij}^{′}} + sin {θ}_{{j}^{′}} sin {ϕ}_{{j}^{′}} {\overline{y}}_{{ij}^{′}} + cos {θ}_{{j}^{′}} {\overline{z}}_{{ij}^{′}} )}{{( {\overline{x}}_{{ij}^{′}}^{2} + {\overline{y}}_{{ij}^{′}}^{2} + {\overline{z}}_{{ij}^{′}}^{2} )}^{5 / 2}} - \\ \frac{cos {θ}_{{j}^{′}}}{{( {\overline{x}}_{{ij}^{′}}^{2} + {\overline{y}}_{{ij}^{′}}^{2} + {\overline{z}}_{{ij}^{′}}^{2} )}^{3 / 2}} ) \\ = \frac{{B}_{r}}{3} {R}^{3} ( \frac{3 {d}_{min}^{2}}{{d}_{min}^{5}} - \frac{1}{{d}_{min}^{3}} ) \\ = \frac{2}{3} \frac{{B}_{r} {R}^{3}}{{d}_{min}^{3}}\end{matrix} , \qquad ( 3.7 )$$

where Eqn. 7 of the Taylor Reference was used to reparameterize the magnetic field in terms of the magnet radius, R. Rearranging (3.7) then gives:

$${d}_{min} = \sqrt[3]{\frac{2}{3} \frac{{B}_{r}}{{B}_{max}}} \cdot R , \qquad ( 3.8 )$$

where dₘᵢₙ and R have the same units as one another, and Bᵣ and Bₘₐₓ have the same units as one another. For a given magnet residual flux density and given sensor, then, the minimum distance that a single magnet can be tracked from that sensor is proportional to the magnet radius.

The LSM9DS1 and LIS3MDL magnetic field sensors (STMicroelectronics, NV) have a full-scale range of Bₘₐₓ=(2⁽¹⁶⁻¹⁾−1)·58·10⁻⁹ T, or 1.9 mT, so with these sensors it may not possible to track a magnet at a distance closer than the dₘᵢₙ corresponding to this Bₘₐₓ. However, the minimum distances corresponding to the recalibration point, Bₘₐₓ=5 mT, as well as to the maximum exposed field, Bₘₐₓ=100 mT (beyond which field level permanent damage to the sensors may occur), can also be considered. For magnets of various residual flux densities, Table 3.1 lists the minimum distances, in multiples of the magnet radius, corresponding to each of the above magnetic field limits when tracking a single magnet.

TABLE 3.1
Minimum Magnet Distance from a Single Magnet to an LSM9DS1 or LIS3MDL
Sensor in Multiples of Magnet Radius, Given Residual Flux Density

| | Residual Flux Density (T) | | | | | | | |
|---|---|---|---|---|---|---|---|---|
| | 0.8 | 0.9 | 1.0 | 1.1 | 1.2 | 1.3 | 1.4 | 1.5 |
| (N-Rating) | (N12) | (N18) | (N24) | (N30) | (N36) | (N42) | (N48) | (N54) |
| Tracking Minimum | 6.55 | 6.81 | 7.06 | 7.29 | 7.50 | 7.70 | 7.90 | 8.08 |
| Recalibration Point | 4.75 | 4.94 | 5.11 | 5.28 | 5.43 | 5.58 | 5.72 | 5.85 |
| Maximum Exposure Point | 1.75 | 1.82 | 1.89 | 1.95 | 2.00 | 2.06 | 2.11 | 2.16 |

Note from the table that the tracking minimum for a single N54 magnet is about eight radii from the center of the magnet to the nearest sensor (seven radii if using a weaker N24 magnet). Thus, the tracking minimum can be a significant constraint on a magnet tracking system, and consideration can be given to the sensor geometry required to meet the design requirements of a given application.

The worst-case scenario given above only remains valid when tracking no more than a single magnet. The tracking boundaries are further limited when tracking multiple magnets. To get the worst-case minimum distance for multiple magnets, Bₘₐₓ can be divided by the number of magnets being tracked. Table 3.2 lists the minimum distances from a pair of magnets to a magnetic field sensor.

TABLE 3.2
Minimum Magnet Distance from a Magnet Pair to an LSM9DS1 or LIS3MDL
Sensor in Multiples of Magnet Radius, Given Residual Flux Density

| | Residual Flux Density (T) | | | | | | | |
|---|---|---|---|---|---|---|---|---|
| | 0.8 | 0.9 | 1.0 | 1.1 | 1.2 | 1.3 | 1.4 | 1.5 |
| (N-Rating) | (N12) | (N18) | (N24) | (N30) | (N36) | (N42) | (N48) | (N54) |
| Tracking Minimum | 8.25 | 8.58 | 8.89 | 9.18 | 9.45 | 9.70 | 9.95 | 10.18 |
| Recalibration Point | 5.98 | 6.22 | 6.44 | 6.65 | 6.84 | 7.03 | 7.21 | 7.37 |
| Maximum Exposure Point | 2.21 | 2.29 | 2.38 | 2.45 | 2.52 | 2.59 | 2.66 | 2.72 |

Note that the tracking minimum distance for two magnets is about two radii larger than the tracking minimum for one magnet. Again, this can be considered a worst-case scenario because it assumes that the two magnets occupy precisely the same location, which is not physically possible for magnets of non-zero diameter (though it can be a valid assumption in an application where two magnets are located on opposite sides of a sensor). When a minimum separation between magnets is enforced in an application, this can be taken into account in the system design when specifying the tracking boundaries.

Finally, though magnetic field disturbances can be compensated for algorithmically, they can still add to the total magnetic field seen by the sensors. Expected maximum magnetic field disturbance levels can be accounted for in these tracking boundary calculations. This can be performed by subtracting the expected maximum magnetic field disturbance from Bₘₐₓ before dividing it by the total number of magnets being tracked. If full magnetic shielding is used, this may not be an issue.

The tables above rely on accurate radius and residual flux density information. Deviations in magnet radius or magnet residual flux density due to manufacturing tolerances can necessitate additional bounding of the tracking volume.

The values in Tables 3.1 and 3.2 depend on which sensor is used. For example, using the LIS2MDL, the tracking minimum corresponds to a larger full-scale range of 4.9152 mT, which is approximately the same as the recalibration point of the LIS3MDL. Furthermore, the LIS2MDL has a maximum exposure point corresponding to 1 Tesla, a magnetic field limit ten times larger than the LIS3MDL can withstand (the magnetic disturbance field is not listed for the LIS2MDL, so the recalibration point cannot be determined from the datasheet). The LIS2MDL, however, has a much lower sampling rate and suffers from increased nonlinearity, and these are also among the sensor characteristics that should be considered in the sensor selection for a particular application.

### 4. Detailed Explanation of Calibration

The following section presents improvements to the methods and systems relating to tracking of magnetic objects found in WO2019/074950, “Method for Neuromechanical and Neuroelectromagnetic Mitigation of Limb Pathology;” the teachings of which are incorporated by reference herein in their entirety.

A method of calibrating a magnetic field sensor array that comprises a plurality of magnets includes three-dimensionally rotating a magnetic field sensor array in a uniform magnetic field and recording data from each of the plurality of sensors. The method further includes calculating a non-rotating transformation of the recorded data using an ellipsoid fit of each of the plurality of sensors and calculating a scaling factor of each of the plurality of sensors based on relative dimensions of the ellipsoid fit. A transformed, scaled dataset for each of the plurality of sensors is obtained through application of the calculated non-rotating transformation and calculated scaling factor. The obtained transformed, scaled datasets are rotated to align, thereby providing for a determination of a relative orientation of each of the plurality of sensors to calibrate the magnetic field sensor array.

Magnetic field sensors are calibrated to account for “hard” and “soft” iron effects. “Hard” iron effects are offsets caused by magnetized components affixed to the same geometric frame as the sensor, and “soft” iron effects are magnetic field redirections caused by temporarily magnetizable components affixed to the same geometric frame as the sensor. When multiple sensors are calibrated simultaneously, the scaling of the sensors relative to one another is also considered, as well as their relative positions and orientations. In this section, methods of addressing the hard and soft iron effects are first provided. Then, recognizing the importance of position and orientation calibration the framework is extended to address how to perform position and orientation calibration on one or more arrays of magnetic field sensors. Previous work has relied upon fabrication tolerances for position and orientation alignment.

Throughout this section, the use of three-axis magnetic field sensors is assumed. Modifications to these methods can be made for single- or two-axis sensors.

4.1 Distortion Correction Via Ellipsoid Fitting with Relative Gains

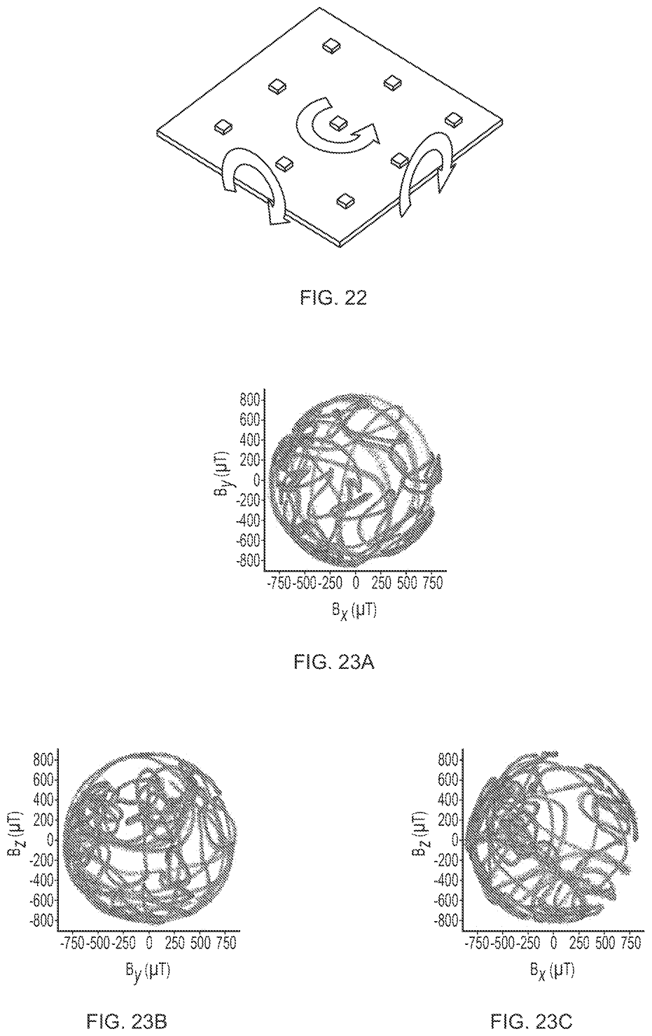

Hard and soft iron effects can be corrected for on a magnetic field sensor by first recording the magnetic field data at the sensor while rotating the sensor in a spatially-uniform ambient magnetic field (see FIG. 22), and then transforming this ellipsoidally-distributed magnetic field data into a sphere centered at the origin. Though it is possible to use just the midpoint between the minimum and maximum magnetic field data points along each axis of the rotated-sensor data to compensate for hard iron offsets, the ellipsoid-to-sphere transformation allows compensation for soft iron effects. An ellipsoid-to-sphere transformation is also less prone to error when the rotated-sensor dataset does not fully cover all possible sensor array orientations. Further, the ellipsoid-to-sphere calibration method has the advantage of accounting for any intrasensor coordinate axis non-orthogonalities due to manufacturing misalignment.

In this section, ellipsoid-to-sphere calibration methods are provided. A straightforward method for scaling the sensor gains by transforming the rotated-sensor-array data from all sensors into a sphere of equivalent size for the calibration of multiple sensors is also provided. With the transformation parameters known, these parameters can then be applied to all future measured magnetic field data, even when the measured field is no longer spatially uniform (e.g., when tracking magnets).

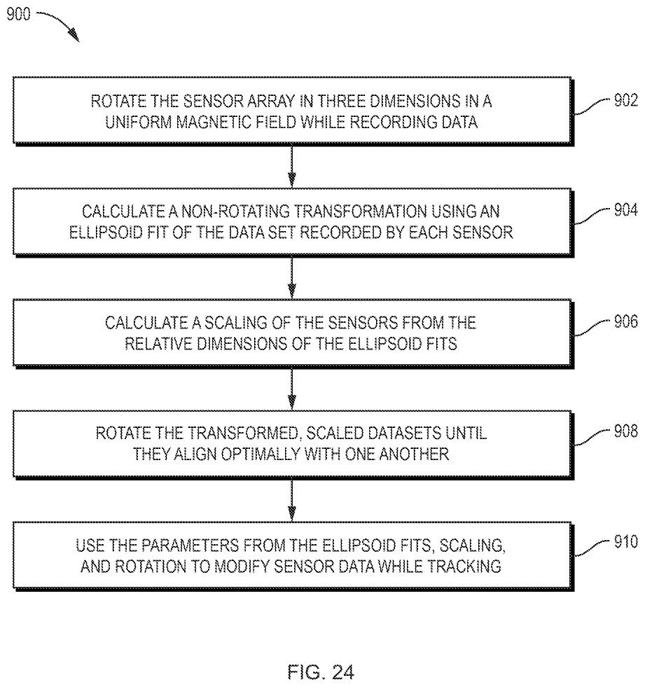

An overview of a calibration method is shown in FIG. 24. The method 900 includes rotating a sensor array in three dimensions in a uniform magnetic field (902) and calculating a non-rotating transformation using an ellipsoid fit of the data set recorded by each sensor (904). A scaling of the sensors from the relative dimensions of the ellipsoid fits is calculated (906). The transformed, scaled datasets are rotated until they align optimally with one another (908), and, using the parameters from the ellipsoid fits, scaling, rotation, sensor data is modified while tracking is performed (910).

To ensure that the field is spatially uniform in the volume used for calibration, calibration can be performed at a distance away from magnets, walls, tables, chairs, floor, etc., such that these items do not affect the magnetic field sampled during the calibration.

The mathematics for computing and implementing the ellipsoid-to-sphere transformation for a magnetic field sensor array are provided below. Following the work from [20], for the ith sensor we seek a symmetric positive-definite matrix

$${A}_{1} = [ \begin{matrix}{A}_{i} & {D}_{i} / 2 & {E}_{i} / 2 \\ {D}_{i} / 2 & {B}_{i} & {F}_{i} / 2 \\ {E}_{i} / 2 & {F}_{i} / 2 & {C}_{i}\end{matrix} ] , \qquad ( 4.1 )$$

a vector bᵢ=[Gᵢ,Hᵢ,Kᵢ]^{T}, and a constant Lᵢ such that, for \tilde{B}ᵢₖ=[\ddot{B}ᵢₖₓ,\ddot{B}_{iky},\ddot{B}_{ikz}]^{T}.

B̅ᵢₖ^{T}AⱼB̅ᵢₖ+bⱼ^{T}B̅ᵢₖ+Lᵢ=0 (4.2)

for all {B̅ᵢₖ}ₖ₌₁^{K} in the calibration data set. This is the parametric equation of an arbitrary ellipsoid and can be expanded as:

$$\frac{1}{3} ( ( {A}_{i} + {B}_{i} + {C}_{i} ) ( {\overline{B}}_{ikx}^{2} + {\overline{B}}_{iky}^{2} + {\overline{B}}_{ikz}^{2} ) + ( {A}_{i} - {C}_{i} ) ( {\overline{B}}_{ikx}^{2} + {\overline{B}}_{iky}^{2} + {\overline{B}}_{ikz}^{2} ) + ( {A}_{i} - {B}_{i} ) ( {\overline{B}}_{ikx}^{2} - 2 {\overline{B}}_{iky}^{2} + {\overline{B}}_{ikz}^{2} ) ) + {D}_{i} {\overline{B}}_{ikx} {\overline{B}}_{iky} + {E}_{i} {\overline{B}}_{ikx} {\overline{B}}_{ikz} + {F}_{i} {\overline{B}}_{iky} {\overline{B}}_{ikz} + {G}_{i} {\overline{B}}_{ikx} + {H}_{i} {\overline{B}}_{iky} + {K}_{i} {\overline{B}}_{ikz} + {L}_{i} = 0 \qquad ( 4.3 )$$

Performing the following linear combinations,

Aᵢ+Bᵢ+Cᵢ=Wᵢ (4.4a)

Aᵢ−Cᵢ=−UᵢWᵢ (4.4b)

Aᵢ−Bᵢ=−VᵢWᵢ (4.4c)

Dᵢ=−4MᵢWᵢ/3 (4.4d)

Eᵢ=−2NᵢWᵢ/3 (4.4e)

Fᵢ=−2pᵢWᵢ/3 (4.4f)

Gᵢ=−QᵢWᵢ/3 (4.4g)

Hᵢ=−RᵢWᵢ/3 (4.4h)

Kᵢ=−SᵢWᵢ/3 (4.4i)

Lᵢ=−TᵢWᵢ/3 (4.4j)

allows (4.3) to be written as

$$\frac{1}{3} ( {W}_{i} ( {\overline{B}}_{ikx}^{2} + {\overline{B}}_{iky}^{2} + {\overline{B}}_{ikz}^{2} ) - {U}_{i} {W}_{i} ( {\overline{B}}_{ikx}^{2} + {\overline{B}}_{iky}^{2} - 2 {\overline{B}}_{ikz}^{2} ) - {V}_{i} {W}_{i} ( {\overline{B}}_{ikx}^{2} - 2 {\overline{B}}_{iky}^{2} + {\overline{B}}_{ikz}^{2} ) - 4 {M}_{i} {W}_{i} {\overline{B}}_{ikx} {\overline{B}}_{iky} - 2 {N}_{i} {W}_{i} {\overline{B}}_{ikx} {\overline{B}}_{ikz} - 2 {P}_{i} {W}_{i} {\overline{B}}_{iky} {\overline{B}}_{ikz} - {Q}_{i} {W}_{i} {\overline{B}}_{ikx} - {R}_{i} {W}_{i} {\overline{B}}_{iky} - {S}_{i} {W}_{i} {\overline{B}}_{ikz} - {T}_{i} {W}_{i} ) = 0 \qquad ( 4.5 )$$

Multiplying both sides by −3/W, and moving the leftmost term to the right-hand side then gives:

$$( {\overline{B}}_{ikx}^{2} + {\overline{B}}_{iky}^{2} - 2 {\overline{B}}_{ikz}^{2} ) {U}_{i} + ( {\overline{B}}_{ikx}^{2} - 2 {\overline{B}}_{iky}^{2} + {\overline{B}}_{ikz}^{2} ) {V}_{i} + 4 {\overline{B}}_{ikx} {\overline{B}}_{iky} {M}_{i} + 2 {\overline{B}}_{ikx} {\overline{B}}_{ikz} {N}_{i} + 2 {\overline{B}}_{iky} {\overline{B}}_{ikz} {P}_{i} + {\overline{B}}_{ikx} {Q}_{i} + {\overline{B}}_{iky} {R}_{i} + {\overline{B}}_{ikz} {S}_{i} + {T}_{i} = ( {\overline{B}}_{ikx}^{2} + {\overline{B}}_{iky}^{2} + {\overline{B}}_{ikz}^{2} ) . \qquad ( 4.6 )$$

The variables in this equation can then be determined by using least-squares to solve

the matrix equation:

[symbol]

$$\text{?} \qquad ( 4.7 )$$

$$\text{?} \text{indicates text missing or illegible when filed}$$

or, most succinctly,

Aᵢs_{LS}_{ᵢ}=eᵢ (4.8)

Once this equation is solved for the values of s_{LSi}, these variables must be mapped back through the inverse of the linear combinations (4.4a)-(4.4j). First, we solve for Aᵢ, Bᵢ, and Cᵢ. From (6.4a)-(6.4c), we have

$$[ \begin{matrix}1 & 1 & 1 \\ 1 & 0 & - 1 \\ 1 & - 1 & 0\end{matrix} ] [ \begin{matrix}{A}_{i} \\ {B}_{i} \\ {C}_{i}\end{matrix} ] = [ \begin{matrix}{W}_{i} \\ - {U}_{i} {W}_{i} \\ - {V}_{i} {W}_{i}\end{matrix} ] \qquad ( 4.9 )$$

so Aᵢ, Bᵢ, and Cᵢ are given by

$$\begin{matrix}[ \begin{matrix}{A}_{i} \\ {B}_{i} \\ {C}_{i}\end{matrix} ] = {[ \begin{matrix}1 & 1 & 1 \\ 1 & 0 & - 1 \\ 1 & - 1 & 0\end{matrix} ]}^{- 1} [ \begin{matrix}{W}_{i} \\ - {U}_{i} {W}_{i} \\ - {V}_{i} {W}_{i}\end{matrix} ] \\ = \frac{1}{3} [ \begin{matrix}1 & 1 & 1 \\ 1 & 1 & - 2 \\ 1 & - 2 & 1\end{matrix} ] [ \begin{matrix}{W}_{i} \\ - {U}_{i} {W}_{i} \\ - {V}_{i} {W}_{i}\end{matrix} ] \\ = \frac{{W}_{i}}{3} [ \begin{matrix}1 & - 1 & - 1 \\ 1 & - 1 & 2 \\ 1 & 2 & - 1\end{matrix} ] [ \begin{matrix}1 \\ {U}_{i} \\ {V}_{i}\end{matrix} ]\end{matrix} \qquad ( 4.1 )$$

Now as described in [13], because we are transforming the data to a sphere, we wish to have tr(Aᵢ)=3, or Aᵢ+Bᵢ+Cᵢ=3, so we have Wᵢ=3, giving

$$[ \begin{matrix}{A}_{i} \\ {B}_{i} \\ {C}_{i}\end{matrix} ] = [ \begin{matrix}1 & - 1 & - 1 \\ 1 & - 1 & 2 \\ 1 & 2 & - 1\end{matrix} ] [ \begin{matrix}1 \\ {U}_{i} \\ {V}_{i}\end{matrix} ] \qquad ( 4.11 )$$

From (4.4d)-(4.4f), Di, Ei, and Fi are then given simply by

Dᵢ=−4Mᵢ (4.12a)

Eᵢ=−2Nᵢ (4.12b)

Fᵢ=−2pᵢ, (4.12c)

and substituting (4.11)-(4.12c) into (4.1) gives

$${A}_{i} = [ \begin{matrix}1 - {U}_{i} - {V}_{i} & - 2 {M}_{i} & - {N}_{i} \\ - 2 {M}_{i} & 1 - {U}_{i} + 2 {V}_{i} & - {P}_{i} \\ - {N}_{i} & - {P}_{i} & 1 + 2 {U}_{i} - {V}_{i}\end{matrix} ] \qquad ( 4.13 )$$

Finally, we note from (4.4j) that the constant Lᵢ=−Tᵢ.

To use the results of the least-squares fit (4.8) in transforming future magnetic field measurements, we first convert from the ellipsoid representation of (4.2), the parametric form

B̅ᵢₖ^{T}AᵢB̅ᵢₖ+bᵢ^{T}B̅ᵢₖ+Lᵢ=0, (4.2 revisited)

to an ellipsoid representation where an offset is applied first, followed by a transformation, the quadric form

(B̅ⱼₖ−vᵢ)^{T}Aᵢ(B̅ᵢₖ−vᵢ)=cᵢ, (4.14)

To represent the ellipsoid in this form, we first modify (4.14) so that we can calculate vᵢ and cᵢ by comparison with (4.2). Because Aᵢ is symmetric (see (4.1)), we can expand (4.14) as

B̅ᵢₖ^{T}AᵢB̅ᵢₖ−B̅ᵢₖ^{T}Aᵢvᵢ−vᵢ−vᵢ^{T}AᵢB̅ᵢₖ+vᵢ^{T}Aᵢvᵢ−cᵢ=0 (4.15a )

B̅ᵢₖ^{T}AᵢB̅ᵢₖ−(B̅ᵢₖ^{T}Aᵢvᵢ)^{T}−vᵢ^{T}AᵢB̅ᵢₖ+vᵢ^{T}Aᵢvᵢ−cᵢ=0 (4.15b)

B̅ᵢₖ^{T}AᵢB̅ᵢₖ−vᵢ^{T}Aᵢ^{T}B̅ᵢₖ−vᵢ^{T}AᵢB̅ᵢₖ+vᵢ^{T}Aᵢvᵢ−cᵢ=0 (4.15c)

B̅ᵢₖ^{T}AᵢB̅ᵢₖ−2vᵢ^{T}AᵢB̅ᵢₖ+vᵢ^{T}Aᵢvᵢ−cᵢ=0 (4.15d)

With (4.15d) and (4.2) in similar form, we can now equate coefficients of like terms to get

$$- 2 {v}_{i}^{T} {A}_{i} = {b}_{i}^{T} \qquad ( 4.16 a )$$

$${( - 2 {v}_{i}^{T} {A}_{i} )}^{T} = {( {b}_{i}^{T} )}^{T} \qquad ( 4.16 b )$$

$$- 2 {A}_{i} {v}_{i} = {b}_{i} \qquad ( 4.16 c )$$

$${v}_{i} = - \frac{1}{2} {A}_{i}^{- 1} {b}_{i} \qquad ( 4.16 d )$$

$${v}_{i} = - \frac{1}{2} {A}_{i}^{- 1} [ \begin{matrix}{G}_{i} \\ {H}_{i} \\ {K}_{i}\end{matrix} ] \qquad ( 4.16 e )$$

$${v}_{i} = - \frac{1}{2} {A}_{i}^{- 1} [ \begin{matrix}{Q}_{i} \\ {R}_{i} \\ {S}_{i}\end{matrix} ] \qquad ( 4.16 f )$$

where the vector vi contains the coordinates of the center of the ellipsoid, which are used to compensate for hard iron effects by offsetting the data. Equating the constant part of (4.14) with the constant part of (4.6) gives

Lᵢ=Vᵢ^{T}Aᵢvᵢ−cᵢ (4.17a)

cᵢ=Tᵢ+vᵢ^{T}Aᵢvᵢ (4.17b)

which we will use to scale the sensor measurements relative to one another. From Eqns. 11 and 12 of [11], this constant cᵢ is representative of the square of the field strength seen by the sensor (i.e., the constant cᵢ is equal to the square of the radius of the sphere after sensitivity scaling has been performed, or cᵢ=rᵢ²). We thus take the square root of this constant to get the sphere radius, rᵢ=√cᵢ. Using the average of the sphere radii across all N sensors,

$${r}_{avg} = \frac{1}{N} {\sum }_{i = 1}^{N} {r}_{i} , \qquad ( 4.18 )$$

a scaling factor can then be computed for each of the sensors,

$${g}_{i} = \frac{{r}_{avg}}{{r}_{i}} . \qquad ( 4.19 )$$

Using the method of [11] to perform a non-rotating transformation (so that the sensors do not rotate relative to one another), we compute the inverse soft-iron matrix as the square root of the ellipsoid matrix

Wᵢ⁻¹=Aᵢ^{½}. (4.20)

Once the calibration is performed, then while collecting data, for all data streams coming in, the data is offset, transformed, and scaled via the equation

\check{B}ᵢ=gᵢSᵢWᵢ⁻¹(\tilde{B}ᵢ−vᵢ), (4.21)

where Sᵢ is the sensor sensitivity given in the sensor's datasheet.

Performing this ellipsoid-to-sphere calibration and applying (4.21) to the calibration data gives a set of spheres which are approximately equal and concentric, as shown in FIGS. 23A-C.

### 4.2 Extension to Arbitrarily Oriented Magnetic Field Sensors

If the magnetic field sensors are not aligned along the same axes (e.g., if they are affixed to a curved rigid surface or to multiple, rigidly-connected but unaligned flat surfaces), the sensor orientations relative to one another (and, importantly, relative to the global magnetic field sensor array coordinate system) can be determined.

To calibrate these relative orientations, the point clouds from the distortion correction calibration, after offset, transformation, and scaling, can be rotated until they are optimally aligned with one another. Because the calibration data is ordered (points are matched at each time point), this alignment can be performed on a point-wise basis instead of resorting to more complex point set registration techniques.

We first define the measured magnetic field B^{\tilde{ }}ᵢ as a rotated version of the sensor data after the distortion correction calibration.

$$( 4.22 )$$

$$\begin{matrix}{\tilde{B}}_{i} {=}^{Δ} {R}_{z} ( {α}_{i} ) {R}_{y} ( {β}_{i} ) {R}_{x} ( {γ}_{i} ) {\tilde{B}}_{i} \\ = [ \begin{matrix}cos {α}_{i} & - s in {α}_{i} & 0 \\ sin {α}_{i} & cos {α}_{i} & 0 \\ 0 & 0 & 1\end{matrix} ] [ \begin{matrix}cos {β}_{i} & 0 & sin {β}_{i} \\ 0 & 1 & 0 \\ - sin {β}_{i} & 0 & cos {β}_{i}\end{matrix} ] [ \begin{matrix}1 & 0 & 0 \\ 0 & cos {γ}_{i} & - s in {γ}_{i} \\ 0 & sin {γ}_{i} & cos {γ}_{i}\end{matrix} ] [ \begin{matrix}{\check{B}}_{ix} \\ {\check{B}}_{i y} \\ {\check{B}}_{iz}\end{matrix} ] \\ = [ \begin{matrix}cos {α}_{i} & - s in {α}_{i} & 0 \\ sin {α}_{i} & cos {α}_{i} & 0 \\ 0 & 0 & 1\end{matrix} ] [ \begin{matrix}cos {β}_{i} & sin {β}_{i} sin {γ}_{i} & sin {β}_{i} cos {γ}_{i} \\ 0 & cos {γ}_{i} & - s in {γ}_{i} \\ - s in {β}_{i} & cos {β}_{i} sin {γ}_{i} & cos {β}_{i} cos {γ}_{i}\end{matrix} ] [ \begin{matrix}{\check{B}}_{ix} \\ {\check{B}}_{i y} \\ {\check{B}}_{iz}\end{matrix} ] \\ = [ \begin{matrix}cos {α}_{i} cos {β}_{i} & \begin{matrix}cos {α}_{i} sin {β}_{i} sin {γ}_{i} - \\ sin {α}_{i} cos {γ}_{i}\end{matrix} & \begin{matrix}cos {α}_{i} sin {β}_{i} cos {γ}_{i} + \\ sin {α}_{i} sin {γ}_{i}\end{matrix} \\ sin {α}_{i} cos {β}_{i} & \begin{matrix}sin {α}_{i} sin {β}_{i} sin {γ}_{i} + \\ cos {α}_{i} cos {γ}_{i}\end{matrix} & \begin{matrix}sin {α}_{i} sin {β}_{i} cos {γ}_{i} - \\ cos {α}_{i} sin {γ}_{i}\end{matrix} \\ - sin {β}_{i} & cos {β}_{i} sin {γ}_{i} & cos {β}_{i} cos {γ}_{i}\end{matrix} ] [ \begin{matrix}{\check{B}}_{ix} \\ {\check{B}}_{i y} \\ {\check{B}}_{iz}\end{matrix} ]\end{matrix} ,$$

where αᵢ, βᵢ, γᵢ are respectively our estimates of the yaw, pitch, and roll of the ith sensor. When no rotation is required (e.g., if the sensors are precisely aligned during manufacturing and assembly), B^{\tilde{ }}ᵢ=B^{\check{ }}ᵢ. A reference sensor is then chosen, which we will denote as sensor n. This reference sensor will define the orientation of the axes of the sensor array coordinate system. This sensor is defined to be oriented, such that αₙ=βₙ=γₙ=0 (and, as a consequence, B^{\tilde{ }}ₙ=B^{\check{ }}ₙ). The rotation parameters of the remaining sensors from the reference sensor are then each given by the solution to the optimization

$${argmin}_{{α}_{i} , {β}_{i} , {γ}_{i}} {\sum }_{k = 1}^{K} {‖ {\tilde{B}}_{nk} - {R}_{i} {\check{B}}_{ik} ‖}_{2} , \qquad ( 4.23 )$$

where we have defined Rᵢ, R_{z}(αᵢ)R_{y}(βᵢ)Rₓ(γᵢ). Upon close inspection of (4.23), it is found that the structure of this problem has previously received particular attention in the Kabsch algorithm (as a special case of the orthogonal Procrustes problem, in turn a special case of Wahba's problem) and can be solved using singular value decomposition [4, 5].

Once the rotation matrix Rᵢ for each sensor is computed, the measured magnetic field value (the final value used for comparison with predicted magnetic field values in the optimization) then becomes

\tilde{B}ᵢ=RᵢgᵢSᵢWᵢ⁻¹(B̅ᵢ−vᵢ). (4.24)

Defining Tᵢ=RᵢgᵢSᵢWᵢ⁻¹ gives

\tilde{B}ᵢ=Tᵢ(B̅ᵢ−vᵢ), (4.25)

or augmenting Tᵢ with −vᵢ to get the affine transformation matrix Tᵢ′=[RᵢgᵢSWᵢ⁻¹|−vᵢ], and using the augmented 4-vector B̅ᵢₖ′=[B̅ᵢₖₓ, B̅_{iky}, B̅_{ikz}, 1]^{T}, we have simply

\tilde{B}ᵢ=Tᵢ′B̅ᵢ′. (4.26)

### 4.3 Extension to Arbitrarily Positioned Magnetic Field Sensors

In Section 3 herein, we assumed that the positions of the magnetic field sensors were previously known while measuring the dipole strength of one or more magnets. Below, we present a method for determining sensor positions using a spherical magnet for position calibration, extending the technique of Section 3.3.

If the sensor positions are unknown, this calibration can simultaneously compute the sensor positions and the dipole's strength. This can be necessary because a magnet's strength is not consistent as viewed by one sensor array versus another.

As before, the magnetic field prediction error is used for the optimization cost function. At the ith sensor and kth timestep, the magnetic field prediction error, Eᵢₖ, is the difference between the measured magnetic field B^{\tilde{ }}ᵢₖ and the predicted magnetic field Bᵢₖ,

Eᵢₖ=Bᵢₖ−\tilde{B}ᵢₖ. (4.27)

Equation (4.27) is identical to (3.4), with the difference being in the way that Bᵢₖ is calculated, as a function of sensor positions.

For each timestep (the kth timestep), the derivatives of the magnetic field prediction errors with respect to each of the sensor position estimates, sᵢₓ, s_{iy}, and s_{iz}, are calculated and placed in a separate submatrix. Because the measured magnetic field B^{\tilde{ }}ᵢₖ of measurement k does not vary with respect to the estimated sensor positions (for example, ∂/∂sₓᵢEᵢₖ=∂/∂sᵢₓBᵢₖ), the derivatives of the cost function can be written as a function of Bᵢₖ alone. Thus, the Jacobian submatrix for sensor position error corresponding to the ith sensor and kth measurement can be calculated as

[symbol]

$$\begin{matrix}{P}_{ik} = [ \begin{matrix}\frac{\partial }{\partial {s}_{ix}} {E}_{ikx} & \frac{\partial }{\partial {s}_{iy}} {E}_{ikx} & \frac{\partial }{\partial {s}_{iz}} {E}_{ikx} \\ \frac{\partial }{\partial {s}_{ix}} {E}_{iky} & \frac{\partial }{\partial {s}_{iy}} {E}_{iky} & \frac{\partial }{\partial {s}_{iz}} {E}_{iky} \\ \frac{\partial }{\partial {s}_{ix}} {E}_{ikz} & \frac{\partial }{\partial {s}_{iy}} {E}_{ikz} & \frac{\partial }{\partial {s}_{iz}} {E}_{ikz}\end{matrix} ] \\ = [ \begin{matrix}\frac{\partial }{\partial {s}_{ix}} {B}_{ikx} & \frac{\partial }{\partial {s}_{iy}} {B}_{ikx} & \frac{\partial }{\partial {s}_{iz}} {B}_{ikx} \\ \frac{\partial }{\partial {s}_{ix}} {B}_{iky} & \frac{\partial }{\partial {s}_{iy}} {B}_{iky} & \frac{\partial }{\partial {s}_{iz}} {B}_{iky} \\ \frac{\partial }{\partial {s}_{ix}} {B}_{ikz} & \frac{\partial }{\partial {s}_{iy}} {B}_{ikz} & \frac{\partial }{\partial {s}_{iz}} {B}_{ikz}\end{matrix} ]\end{matrix} \qquad ( 4.28 )$$

The columns of derivatives in (4.28) are written in terms of sᵢₓ, s_{iy}, and s_{iz}, but Bᵢ in Eqns. 8a and 8c of the Taylor Reference is written in terms of x⁻ᵢⱼ, y⁻ᵢⱼ, and z⁻ᵢⱼ. From the definitions of x⁻ᵢⱼ, y⁻ᵢⱼ, and z⁻ᵢⱼ in (2.3) we see that x⁻ᵢⱼ, y⁻ᵢⱼ, and z⁻ᵢⱼ are functions of sᵢₓ, s_{iy}, and s_{iz}, respectively. The chain rule can thus be used for all M magnets at the kth measurement as

[symbol]

$$\frac{\partial {B}_{ik}}{\partial {s}_{ix}} = {\sum }_{{j}^{′} = 1}^{M} ( \frac{\partial {B}_{ik}}{\partial {\overline{x}}_{{ij}^{′} k}} \frac{\partial {\overline{x}}_{{ij}^{′} k}}{\partial {s}_{ix}} + \frac{\partial {B}_{ik}}{\partial {\overline{y}}_{{ij}^{′} k}} \frac{\partial {\overline{y}}_{{ij}^{′} k}}{\partial {s}_{ix}} + \frac{\partial {B}_{ik}}{\partial {\overline{z}}_{{ij}^{′} k}} \frac{\partial {\overline{z}}_{{ij}^{′} k}}{\partial {s}_{ix}} ) = {\sum }_{{j}^{′} = 1}^{M} ( \frac{\partial {B}_{ik}}{\partial {\overline{x}}_{{ij}^{′} k}} ( 1 ) + \frac{\partial {B}_{ik}}{\partial {\overline{y}}_{{ij}^{′} k}} ( 0 ) + \frac{\partial {B}_{ik}}{\partial {\overline{z}}_{{ij}^{′} k}} ( 0 ) ) \qquad ( 4.29 )$$

for sᵢₓ, and similarly for s_{iy} and s_{iz} to get

[symbol]

$${P}_{ij} = {\sum }_{{j}^{′} = 1}^{M} \text{?} \qquad ( 4.3 )$$

$$\text{?} \text{indicates text missing or illegible when filed}$$

in which we note that the derivative for each element is now computed as a summation over all M terms of Eqn. 8 (of the Taylor Reference). In other words, each of the elements of Pᵢₖ is a negated summation of the M elements of J corresponding to the ith sensor and kth measurement along each given axis. However, unless some advantage is found for position calibration using multiple magnets, the use of a single magnet (M=1) can be preferred, and in this case no summation is required.

The submatrix of the Jacobian of magnetic field prediction errors corresponding to all sensor position estimates at measurement k is then given by

$${P}_{k} = [ \begin{matrix}{P}_{1 k} & 0 & … & 0 \\ 0 & {P}_{2 k} & … & 0 \\ ⋮ & ⋮ & ⋱ & ⋮ \\ 0 & 0 & … & {P}_{Nk}\end{matrix} ] . \qquad ( 4.31 )$$

The dipole measurement matrix can then be augmented to give us the sensor position calibration matrix

$$\tilde{\tilde{J}} = [ \begin{matrix}{J}_{1} & 0 & … & 0 & {M}_{1} & {P}_{1} \\ 0 & {J}_{2} & … & 0 & {M}_{2} & {P}_{2} \\ ⋮ & ⋮ & ⋱ & & ⋮ & ⋮ \\ 0 & 0 & … & {J}_{K} & {M}_{K} & {P}_{K}\end{matrix} ] \qquad ( 4.32 )$$

where K is the number of samples taken at different time steps.

Note that this derivation above does not tell a perfect picture, since either a few distinct positions must be known (or some combination of known position and/or orientation for multiple points), or one of the sensors can be used as the “origin” sensor (likely a simpler option). Say that the reference sensor is sensor n. Then, Pₖ is given by

$${P}_{k} = [ \begin{matrix}{P}_{1 k} & 0 & … & 0 & 0 & … & 0 \\ 0 & {P}_{2 k} & … & 0 & 0 & … & 0 \\ ⋮ & ⋮ & ⋱ & ⋮ & ⋮ & ⋱ & ⋮ \\ 0 & 0 & … & {P}_{( n - 1 ) k} & 0 & … & 0 \\ 0 & 0 & … & 0 & 0 & … & 0 \\ 0 & 0 & … & 0 & {P}_{( n + 1 ) k} & … & 0 \\ ⋮ & ⋮ & ⋱ & ⋮ & ⋮ & ⋱ & ⋮ \\ 0 & 0 & … & 0 & 0 & … & {P}_{Nk}\end{matrix} ] \qquad ( 4.33 )$$

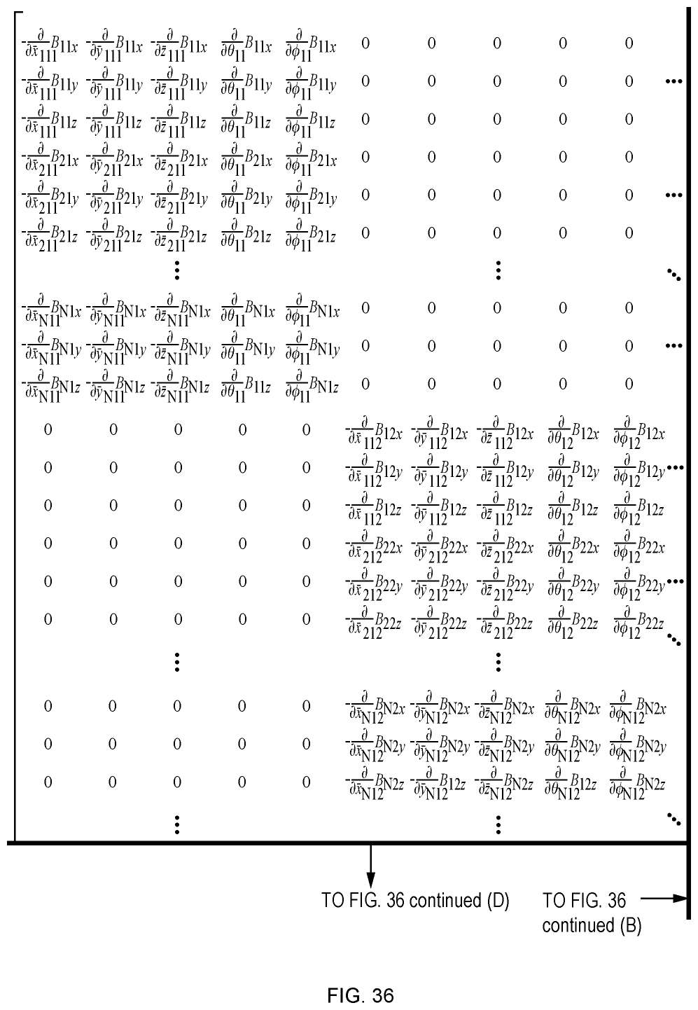

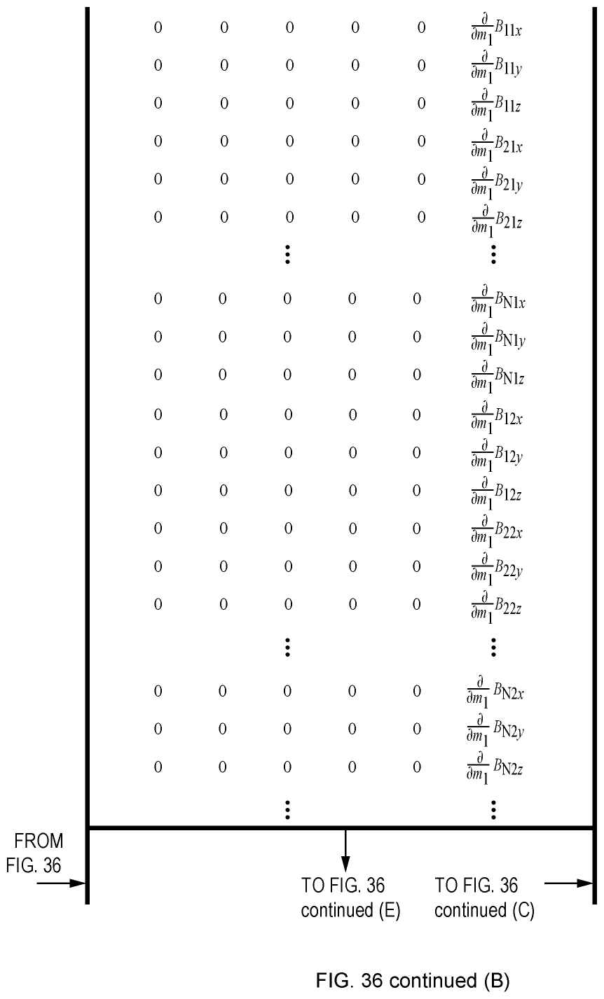

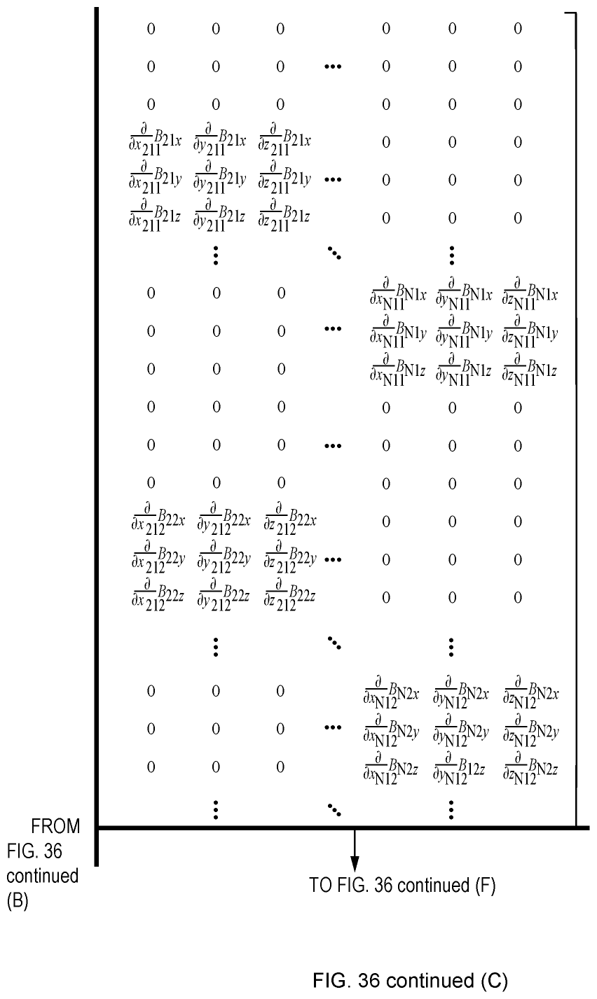

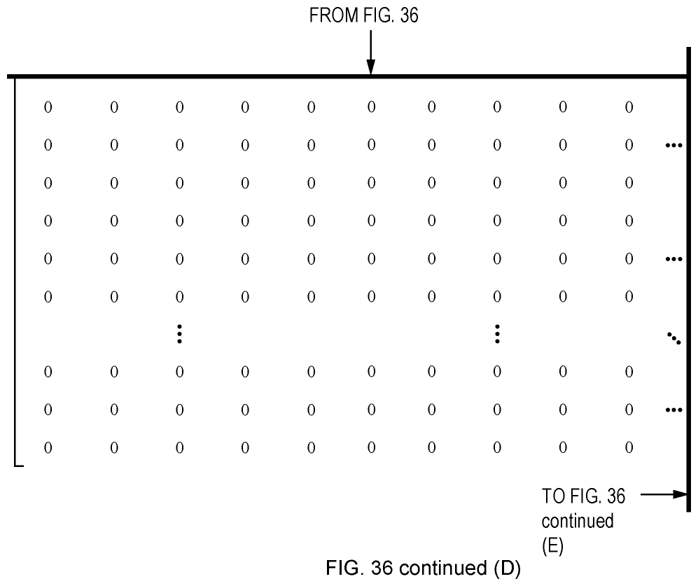

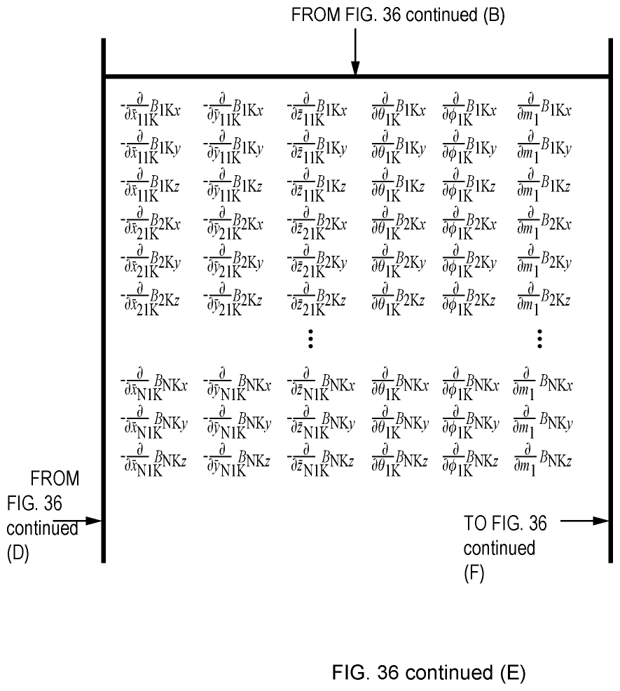

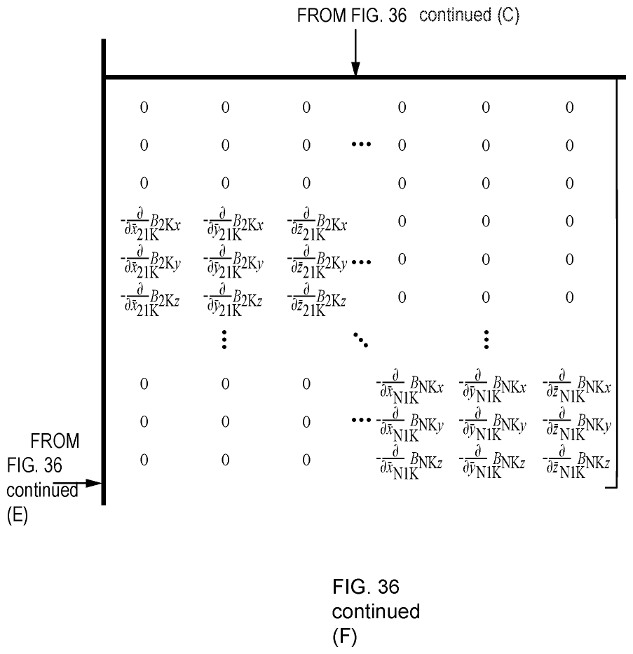

Using the notation given above and letting n=1, and when calibrating positions with a single magnet, the sensor position calibration matrix appears in expanded form as shown in FIG. 36.

### 4.4 Further Technical Considerations

Throughout this section, the assumption was made that the sensors are linear. This assumption had no bearing on the given approach to calibrating sensor orientations and positions, or on the given method for measuring dipole strengths. However, nonlinearity of the sensors (e.g., of the LIS3MDL or LSM9DS1 magnetic tunnel junctions) can warp the magnetic field measurements, and soft iron saturation can warp the actual magnetic field at the sensor. Though it was suggested in the Taylor Reference that the magnetization inhomogeneity of spherical permanent magnets can contribute to the tracking error observed when magnets are close to the sensing array, tracking errors at near-field caused by sensor nonlinearity are likely greater in magnitude. Upon inspection of the datasheet for the LIS3MDL (which is equivalent to the LSM9DS1), when sensing a 600 μT magnetic field with a full-scale range of 1200 μT, the typical specification of the nonlinearity is 0.12% of full-scale range, or 1.44 μT, roughly half the RMS noise of the sensor. This error can be even more substantial when sensing larger magnetic fields, as well as when sensing with a full-scale range of 1600 μT. It may thus be valuable to consider implementing nonlinearity calibration on the sensors to increase tracking accuracy at near-field. However, such calibration techniques can be non-trivial to implement, and in combining and extending the strategies above, can potentially adversely affect tracking latency.

In the Taylor Reference, spike noise was filtered out using a three-point median filter. It was originally thought that the spike noise was quantum mechanical shot noise from the magnetic tunnel junction of the LSM9DS1 tunneling magnetoresistors used in the Taylor Reference. With further investigation, however, it was discovered that this spike noise was just due to communication issues, which was corrected by enabling the block data update bit on the LIS3MDL tunneling magnetoresistors described in Example 1 herein.

Upon further inspection of the LIS3MDL datasheet, the LIS3MDL has a temperature sensor output change vs. temperature of 8 LSB/° C. Thus, a single degree change in temperature can cause a magnetic field sensing error of 464 nT, greater even than the RMS noise of 410 nT reported for the sensor. To account for these temperature effects, as much as possible the sensors were calibrated at the same temperature as was expected in all experiments. However, a swing in temperature of, for instance, the ten degrees Celsius difference between room temperature and the temperature of a turkey leg, can easily skew the magnetic field readings by 11 times the RMS noise. If this skewing is normally distributed, when using many sensors, this should have a negligible effect on the tracking accuracy in our application.

In the Taylor Reference a backwards labeling of the magnetic field sensing axes in the LSM9DS1 datasheet was erroneously followed. Because, mathematically, the reversal of all three axes is equivalent to the tracking of magnets that are reversed in polarity, this went undetected until the seventh-generation sensor array employed in Example 1, when a bar magnet was used to aid in setting up the sensor array coordinate axes. Thus, in the Taylor Reference, the magnets were tracked as if they were aligned south-pole-up in the tracking wands, instead of north-pole-up as intended and physically implemented, much to our confusion during tracking, but without any effect on the validity of the results.

### 5. Extension of Disturbance Rejection to Higher Order Terms of the Disturbance

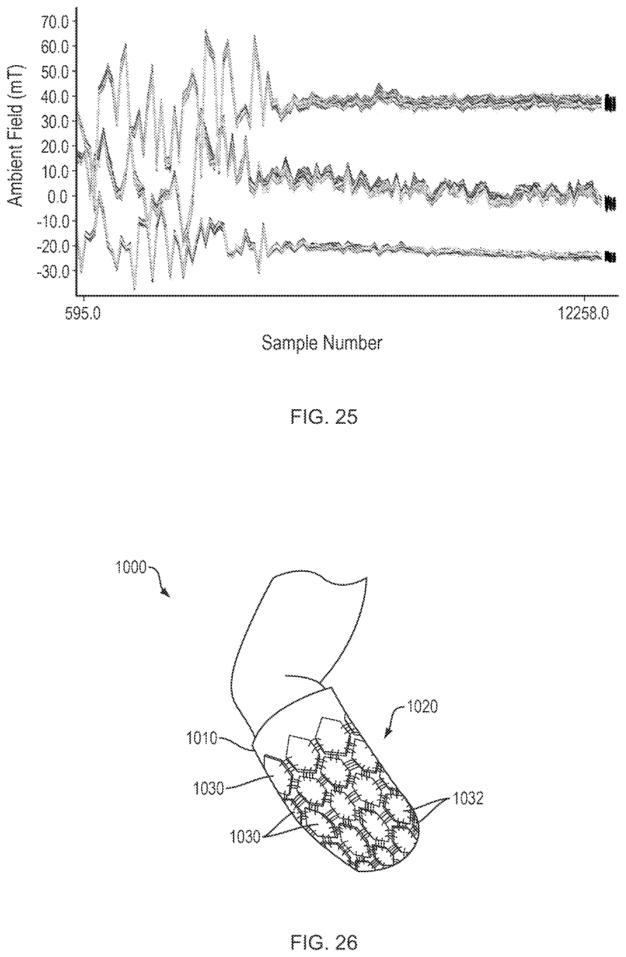

As discussed in the Taylor Reference, magnetic disturbance fields can be compensated for by tracking the disturbance fields as part of a magnet tracking optimization algorithm. When objects causing or warping disturbance fields are sufficiently far away, the disturbance presents as a spatially-uniform field. For example, when moving a magnetic compass side-to-side far away from any magnets, the reading on the compass changes very little, indicating that the field is relatively consistent within the local volume traversed. For example, FIG. 25 shows magnetic field fluctuations as measured inside a train when being passed by another train traveling in the opposite direction on an adjacent track. Notice that the corresponding x, y, and z axes across all of the sensors move up and down together. In other words, a uniform disturbance field assumption can reject all far-field magnetic disturbances, regardless of the source.

However, as these objects come nearer to the magnetic field sensor array, the disturbance becomes increasingly non-uniform. That is, the disturbance presents as a spatial gradient. Even in FIG. 25, more careful inspection reveals that the measurements across equivalent axes are not precisely equal. These nonuniform disturbances can be compensated for in much the same way that non-uniform disturbances were compensated for in the Taylor Reference, by including the spatial gradient components of the disturbance field in the tracking parameters.

### 5.1 Compensation for the Spatial Gradient of the Disturbance

Modifying Eqn. 8 of the Taylor Reference to include the spatial gradient of the disturbance field gives

$${B}_{i} = G + {s}_{i} \cdot 𝒢 + {\sum }_{{j}^{′} = 0}^{{j}^{′} = M} ( \frac{3 {r}_{{ij}^{′}} ( {\overline{m}}_{{j}^{′ T}} \cdot {r}_{{ij}^{′}} )}{{r}_{{ij}^{′}}^{5}} - \frac{{\overline{m}}_{{j}^{′}}}{{r}_{{ij}^{′}}^{3}} ) , \qquad ( 5.1 )$$

where G is the spatially-uniform (zeroth order) component of the disturbance field and G is a tensor containing the first-order spatial gradient information. In expanded form, this is shown as

$${B}_{i} = G + [ \begin{matrix}{s}_{ix} & {s}_{iy} & {s}_{iz}\end{matrix} ] [ \begin{matrix}{G}_{{x}_{x}} & {G}_{{y}_{x}} & {G}_{{z}_{x}} \\ {G}_{{x}_{y}} & {G}_{{y}_{y}} & {G}_{{z}_{y}} \\ {G}_{{x}_{z}} & {G}_{{y}_{z}} & {G}_{{z}_{z}}\end{matrix} ] + {\sum }_{{j}^{′} = 0}^{{j}^{′} = M} ( \frac{3 {r}_{{ij}^{′}} ( {\overline{m}}_{{j}^{′ T}} \cdot {r}_{{ij}^{′}} )}{{r}_{{ij}^{′}}^{5}} - \frac{{\overline{m}}_{{j}^{′}}}{{r}_{{ij}^{′}}^{3}} ) , \qquad ( 5.2 )$$

where G_{xy} is the first-spatial-derivative with respect toy of the x-directed disturbance field. Note that, fully generalizing, G_{xy} and G_{yx} represent distinct properties. Now G introduces nine additional parameters to the optimization, so a new submatrix is concatenated to the Jacobian matrix

$$\begin{matrix}{Dxyz}_{i} = [ \begin{matrix}\frac{\partial }{\partial {G}_{{x}_{x}}} {B}_{ix} & \frac{\partial }{\partial {G}_{{y}_{x}}} {B}_{ix} & \frac{\partial }{\partial {G}_{{z}_{x}}} {B}_{ix} & \frac{\partial }{\partial {G}_{{x}_{y}}} {B}_{ix} & \frac{\partial }{\partial {G}_{{y}_{y}}} {B}_{ix} & \frac{\partial }{\partial {G}_{{z}_{y}}} {B}_{ix} & \frac{\partial }{\partial {G}_{{x}_{z}}} {B}_{ix} & \frac{\partial }{\partial {G}_{{y}_{z}}} {B}_{ix} & \frac{\partial }{\partial {G}_{{z}_{z}}} {B}_{ix} \\ \frac{\partial }{\partial {G}_{{x}_{x}}} {B}_{iy} & \frac{\partial }{\partial {G}_{{y}_{x}}} {B}_{iy} & \frac{\partial }{\partial {G}_{{z}_{x}}} {B}_{iy} & \frac{\partial }{\partial {G}_{{x}_{y}}} {B}_{iy} & \frac{\partial }{\partial {G}_{{y}_{y}}} {B}_{iy} & \frac{\partial }{\partial {G}_{{z}_{y}}} {B}_{iy} & \frac{\partial }{\partial {G}_{{x}_{z}}} {B}_{iy} & \frac{\partial }{\partial {G}_{{y}_{z}}} {B}_{iy} & \frac{\partial }{\partial {G}_{{z}_{z}}} {B}_{iy} \\ \frac{\partial }{\partial {G}_{{x}_{x}}} {B}_{iz} & \frac{\partial }{\partial {G}_{{y}_{x}}} {B}_{iz} & \frac{\partial }{\partial {G}_{{z}_{x}}} {B}_{iz} & \frac{\partial }{\partial {G}_{{x}_{y}}} {B}_{iz} & \frac{\partial }{\partial {G}_{{y}_{y}}} {B}_{iz} & \frac{\partial }{\partial {G}_{{z}_{y}}} {B}_{iz} & \frac{\partial }{\partial {G}_{{x}_{z}}} {B}_{iz} & \frac{\partial }{\partial {G}_{{y}_{z}}} {B}_{iz} & \frac{\partial }{\partial {G}_{{z}_{z}}} {B}_{iz}\end{matrix} ] \\ = [ \begin{matrix}{s}_{ix} & 0 & 0 & {s}_{iy} & 0 & 0 & {s}_{iz} & 0 & 0 \\ 0 & {s}_{ix} & 0 & 0 & {s}_{iy} & 0 & 0 & {s}_{iz} & 0 \\ 0 & 0 & {s}_{ix} & 0 & 0 & {s}_{iy} & 0 & 0 & {s}_{iz}\end{matrix} ] \\ = [ \begin{matrix}{s}_{ix} {I}_{3} & {s}_{iy} {I}_{3} & {s}_{iz} {I}_{3}\end{matrix} ]\end{matrix} \qquad ( 5.3 )$$

The further augmented Jacobian matrix including the gradient of the disturbance field, which we'll call J^{\hat{ }}\hat{ }, is then given by

$$\hat{\hat{J}} = [ \begin{matrix}{J}_{11} & {J}_{12} & … & {J}_{1 M} & {I}_{3} & {s}_{1 x} {I}_{3} & {s}_{1 y} {I}_{3} & {s}_{1 z} {I}_{3} \\ {J}_{21} & {J}_{22} & … & {J}_{2 M} & {I}_{3} & {s}_{2 x} {I}_{3} & {s}_{2 y} {I}_{3} & {s}_{2 z} {I}_{3} \\ ⋮ & ⋮ & ⋱ & ⋮ & ⋮ & ⋮ & ⋮ & ⋮ \\ {J}_{N 1} & {J}_{N 2} & … & {J}_{NM} & {I}_{3} & {s}_{Nx} {I}_{3} & {s}_{Ny} {I}_{3} & {s}_{Nz} {I}_{3}\end{matrix} ] \qquad ( 5.4 )$$

Note that some of these elements are not meaningful when working with a planar array, so they may not be used in practice when the sensors lie on a single flat circuit board.

One could continue to higher-order dimensions, with a 27-element tensor for the second-order, an 81-element tensor for the third-order, and further, but the added disturbance tracking sophistication can degrade tracking performance, especially when using a smaller number of sensors.

At some level, when an intricately complicated disturbance field is expected, alternative solutions can be considered. For instance, additional magnetic dipoles can be tracked (with the earth itself being tracked as a large dipole far away). Alternatively, lightweight strategies for implementing magnetic shielding in ways that eliminate or simplify the magnetic disturbance can be employed.

### 5.1.1 Simplification of the Spatial Gradient Disturbance Compensation

On the other hand, if we allow the assumption that the source of the disturbance(s) is dipolar in nature (e.g., that the magnetic field is not being generated by electric current in, for instance, a long wire), we can apply the conditions of symmetry and zero-trace to G, as in [9], to simplify the disturbance tracking, as

[symbol]

$${B}_{i} = {G}_{0} + [ \begin{matrix}{s}_{ix} & {s}_{iy} & {s}_{iz}\end{matrix} ] [ \begin{matrix}{G}_{{x}_{x}} & {G}_{{x}_{y}} & {G}_{{z}_{x}} \\ {G}_{{x}_{y}} & {G}_{{y}_{y}} & {G}_{{z}_{y}} \\ {G}_{{z}_{x}} & {G}_{{z}_{y}} & - {G}_{{x}_{x}} - {G}_{{y}_{y}}\end{matrix} ] + {\sum }_{{j}^{′} = 0}^{{j}^{′} = M} ( \frac{3 {r}_{{ij}^{′}} ( {\overline{m}}_{{j}^{′ T}} \cdot {r}_{{ij}^{′}} )}{{r}_{{ij}^{′}}^{5}} - \frac{{\overline{m}}_{{j}^{′}}}{{r}_{{ij}^{′}}^{3}} ) , \qquad ( 5.5 )$$

where G can now be described by five parameters. We then have the reduced-size disturbance-gradient Jacobian submatrix

[symbol]

$$\text{?}$$

$$\text{?} \text{indicates text missing or illegible when filed}$$

which further simplifies the Jacobian matrix to

[symbol]

$$\hat{J} = [ \begin{matrix}{J}_{11} & {J}_{12} & … & {J}_{1 M} & {I}_{3} & {\overline{D}}_{{xyz}_{1}} \\ {J}_{21} & {J}_{22} & … & {J}_{2 M} & {I}_{3} & {\overline{D}}_{{xyz}_{2}} \\ ⋮ & ⋮ & ⋱ & ⋮ & ⋮ & ⋮ \\ {J}_{N 1} & {J}_{N 2} & … & {J}_{NM} & {I}_{3} & {\overline{D}}_{{xyz}_{N}}\end{matrix} ] . \qquad ( 5.7 )$$

### 5.2 Flexible Magnetic Shielding

In magnetomicrometry, as discussed above, uniform, linear, and even nonlinear magnetic disturbance fields can be compensated for using software. However, if the disturbance field is linear or nonlinear, this complicates the software, and becomes increasing more difficult if the field needs to be described by higher order terms. Further, if the magnetic disturbance is large, there is the potential for damage to the magnetic field sensors.

As discussed above, one solution to the disturbance problem is to introduce magnetic shielding. Past use of the shielding technique for tracking magnetic beads in the body have used the full-room (Faraday cage) shielding, or placement of a cylindrical shield, open on both sides, around the part of the body wherein magnetic beads are to be tracked.

The full-room shielding is limited because it does not allow for disturbance compensation outside the room, so it isn't a portable solution. The cylindrical solution is limited because it must be large in order to fit the limb, and generalizing it requires creating a solution that is unnecessarily large for wrapping around, for instance, extremities that are smaller.

Devices and methods for shielding which surpasses these limitations and allows for items of variable geometries to be optimally shielded are provided. Two examples of wearable shielding assemblies 1000, and 1050 are described below. In both of these examples, the shielding is external to the magnet-sensor system. In either of these examples, the shielding can be embedded in the socket as part of a lamination or placed around the outside of the socket.

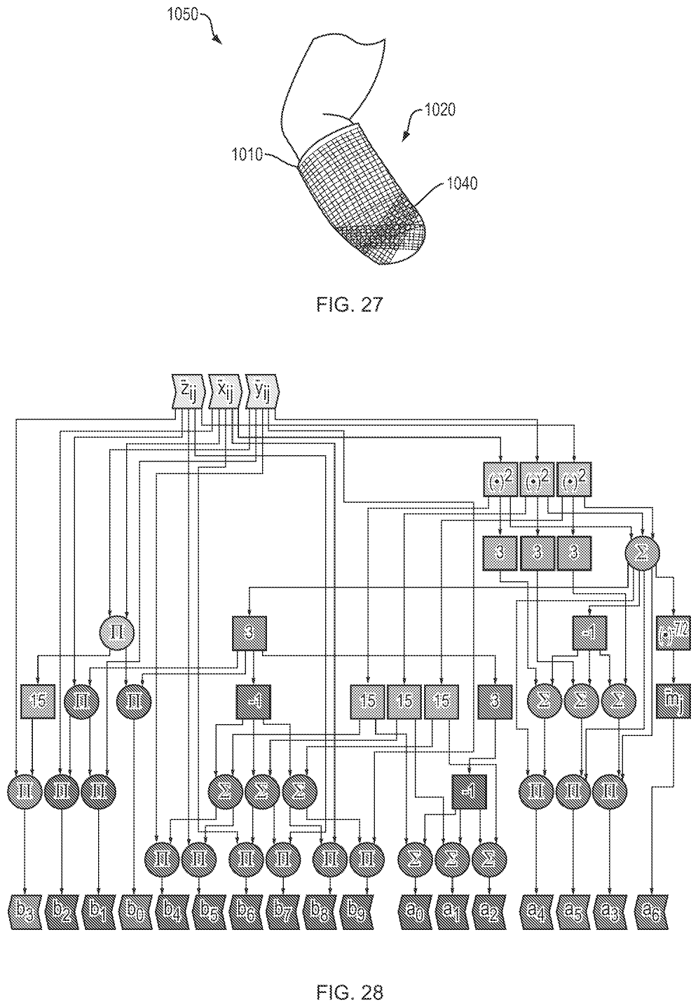

As illustrated in FIGS. 26 and 27, a wearable shielding assembly 1000, 1050 can include an array of sensors (e.g., sensors 106a-e, or magnetic field sensors 208a-b, sensors not visible in FIGS. 26 and 27) configured to detect a state change of at least magnetic target implanted at a tissue (e.g., targets 101, 102, 201, 202, targets not visible in FIGS. 26 and 27). The shielding assembly further includes a wearable receptacle 1010 within which the array of sensors can be disposed. A geometrically-reconfigurable material 1020 is disposed about the wearable or is integral with the wearable. Examples of geometrically-reconfigurable material are provided below. The geometrically-reconfigurable material can be flexible and can, optionally, be rigidly affixed to the wearable receptacle after application.

### 5.2.1 Tiled Magnetic Shielding

FIG. 26 illustrates an example shielding 1000 that includes a plurality of ferromagnetic tiles 1030. In this example, multiple ferromagnetic tiles can be connected together to create a full magnetic shield. The connections between the multiple tiles in this invention can be made by connectors 1032, such as, for example snap fasteners, hooks, buttons, hook-and-loop fasteners (such as Velcro, or 3M Dual-Lock fasteners), glue, suture threads, knots, and elastic bands. These connections can be established prior to use or can be adjusted by the user. The ferromagnetic tiles in this invention can be thin or thick and may be multi-material (multi-layered), including MuMetal® (Magnetic Shield Corp., IL), steel, and other ferromagnetic materials. These tiles can be a repeated size and shape but may also vary in shape and size to provide for a customizable fit.

Unlike in lamellar armor, the individual tiles, called plates or scales in lamellar armor, need not overlap. Thus, the lacing or other means of connection between the scales can be made of a flexible material, with gaps being allowed between tiles when the shielding is stretched. Alternatively, the tiles may be attached to, for example, the outside of a socket, using a method of attachment such as stickers, glue, epoxy or Velcro disposed on a back side of each of the tiles, in place of or in addition to the connections directly between the tiles.

### 5.2.2 Layered Metal Mesh for Magnetic Shielding

FIG. 27 illustrates another example of shielding 1050 that includes a ferromagnetic mesh 1040. In this example, metal mesh (also known as hardware cloth or chicken wire, depending upon material and context) is applied around the region where magnets targets are being tracked. Application can occur as a single wrap around the region, for instance, around a lower leg as illustrated, or can occur as multiple wrappings of one material or of multiple different materials. For example, one or more layers of MuMetal mesh can be applied as a base layer for the shielding, followed by steel chicken wire for additional outer layers. Full coverage of the metal mesh is not necessary for magnetic shielding to occur, so a small number of wraps can be used to minimize the weight and volume of the shielding. The wrap can be incorporated into the composite structure of a prosthetic socket, or incorporated into a composite structure of an orthotic interface between an exoskeleton and the human body.

### 6. Common Subexpression Elimination, Practical Notes, and Further Improvements

The following section presents improvements to the methods and systems relating to tracking of magnetic objects found in WO2019/074950, “Method for Neuromechanical and Neuroelectromagnetic Mitigation of Limb Pathology;” the teachings of which are incorporated by reference herein in their entirety.

A method of tracking one or more permanent magnets includes detecting a signal from each of the one or more permanent magnets, calculating an analytically-derived Hessian matrix with respect to the detected signals, and determining a state of each of the one or more permanent magnets based on the calculated Hessian matrix.

The method can further include calculating a predicted magnetic field value for each of the one or more permanent magnets and calculating a Jacobian matrix with respect to the detected signals. Determining the state of each of the one or more permanent magnets can be further based on the calculated predicted magnetic field values and the calculated Jacobian matrix. Calculation of the Hessian matrix, the Jacobian matrix, and the predicted magnetic field values can be performed simultaneously using common subexpression elimination. The calculation of the Hessian matrix can be parallelized.

### 6.1 Common Subexpression Elimination

There are many common subexpressions encountered when calculating the magnetic field prediction error and its derivatives. As discussed in the Taylor Reference, to increase the speed of the algorithm, common subexpression elimination was implemented, evaluating repeated subexpressions only once whenever possible. Noting that the implementation of common subexpression elimination is best hand-optimized [15], the common subexpression elimination solution used was first developed on paper before being implemented on a computer. Local variables were used in the code whenever possible, and the number of variables used was kept as low as possible to increase the use of fast-access registers for memory storage (a technique most efficiently implemented in a compiled language). This section describes the structure of subexpression calculations that can be used to achieve low-latency tracking.

In the equations below, some expressions represent variable declarations, some expressions indicate self-referential variable modification, and some expressions represent non-self-referential variable reassignment. Some final variable assignments to variables were used in the final step of the calculation, and some final variable assignments to variables were not used in the final step of the calculation. All calculations were executed from top to bottom, then from left to right (fully computing each column before moving to the next one).

All equations were verified for accuracy using a symbolic solver.

### 6.1.1 Common Subexpression Elimination of the Jacobian Matrix Elements

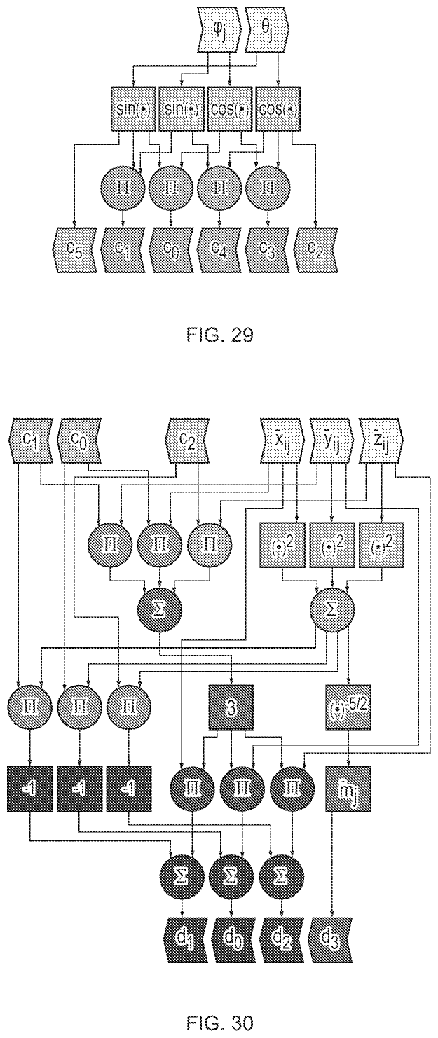

For each submatrix of the Jacobian, subexpressions were calculated, as shown in FIGS. 28 and 29, as

[symbol]

$$\begin{matrix}{a}_{0} = {\overline{x}}_{ij}^{2} \\ {a}_{1} = {\overline{y}}_{ij}^{2} \\ {a}_{2} = {\overline{z}}_{ij}^{2} \\ {a}_{7} = {a}_{0} + {a}_{1} + {a}_{2} \\ {a}_{3} = {a}_{7} ( 3 {a}_{0} - {a}_{7} ) \\ {a}_{4} = {a}_{7} ( 3 {a}_{1} - {a}_{7} ) \\ {a}_{5} = {a}_{7} ( 3 {a}_{2} - {a}_{7} )\end{matrix} \begin{matrix}{a}_{0} = 15 {a}_{0} \\ {a}_{1} = 15 {a}_{1} \\ {a}_{2} = 15 {a}_{2} \\ {b}_{3} = {\overline{x}}_{ij} {\overline{y}}_{ij} \\ {a}_{8} = 3 {a}_{7} \\ {b}_{0} = {b}_{3} {a}_{8} \\ {b}_{2} = {\overline{z}}_{ij} {a}_{8} \\ {b}_{5} = {a}_{0} - {a}_{8} \\ {b}_{7} = {a}_{1} - {a}_{8} \\ {b}_{9} = {a}_{2} - {a}_{8}\end{matrix} \begin{matrix}{a}_{8} = 3 {a}_{8} \\ {a}_{0} = {a}_{0} - {a}_{8} \\ {a}_{1} = {a}_{1} - {a}_{8} \\ {a}_{2} = {a}_{2} - {a}_{8} \\ {a}_{6} = {\overline{m}}_{j} {a}_{7}^{- 7 / 2}\end{matrix}$$

$$\begin{matrix}{b}_{1} = {\overline{y}}_{ij} {b}_{2} \\ {b}_{2} = {\overline{x}}_{ij} {b}_{2} \\ {b}_{3} = 15 {b}_{3} {\overline{z}}_{ij} \\ {b}_{4} = {\overline{y}}_{ij} {b}_{5} \\ {b}_{5} = {\overline{z}}_{ij} {b}_{5} \\ {b}_{6} = {\overline{x}}_{ij} {b}_{7} \\ {b}_{7} = {\overline{z}}_{ij} {b}_{7} \\ {b}_{8} = {\overline{x}}_{ij} {b}_{9} \\ {b}_{9} = {\overline{y}}_{ij} {b}_{9}\end{matrix} \begin{matrix}{c}_{2} = {θ}_{j} \\ {c}_{3} = {ϕ}_{j} \\ {c}_{4} = sin {c}_{3} \\ {c}_{3} = cos {c}_{3} \\ {c}_{5} = sin {c}_{2} \\ {c}_{2} = cos {c}_{2} \\ {c}_{0} = {c}_{8} {c}_{3} \\ {c}_{1} = {c}_{5} {c}_{4} \\ {c}_{3} = {c}_{3} {c}_{2} \\ {c}_{4} = {c}_{4} {c}_{2}\end{matrix}$$

giving variables equal to the expressions

[symbol]

$$\begin{matrix}{a}_{0} = 3 ( 2 {\overline{x}}_{ij}^{2} - 3 {\overline{y}}_{ij}^{2} - 3 {\overline{z}}_{ij}^{2} ) \\ {a}_{1} = 3 ( - 3 {\overline{x}}_{ij}^{2} + 2 {\overline{y}}_{ij}^{2} - 3 {\overline{z}}_{ij}^{2} ) \\ {a}_{2} = 3 ( - 3 {\overline{x}}_{ij}^{2} - 3 {\overline{y}}_{ij}^{2} + 2 {\overline{z}}_{ij}^{2} ) \\ {a}_{3} = ( {\overline{x}}_{i}^{2} + {\overline{y}}_{i}^{2} + {\overline{z}}_{i}^{2} ) ( 2 {\overline{x}}_{ij}^{2} - {\overline{y}}_{ij}^{2} - {\overline{z}}_{ij}^{2} ) \\ {a}_{4} = ( {\overline{x}}_{i}^{2} + {\overline{y}}_{i}^{2} + {\overline{z}}_{i}^{2} ) ( - {\overline{x}}_{ij}^{2} + 2 {\overline{y}}_{ij}^{2} - {\overline{z}}_{ij}^{2} ) \\ {a}_{5} = ( {\overline{x}}_{i}^{2} + {\overline{y}}_{i}^{2} + {\overline{z}}_{i}^{2} ) ( - {\overline{x}}_{ij}^{2} - {\overline{y}}_{ij}^{2} + 2 {\overline{z}}_{ij}^{2} ) \\ {a}_{6} = {{\overline{m}}_{j} ( {\overline{x}}_{ij}^{2} + {\overline{y}}_{ij}^{2} + {\overline{z}}_{ij}^{2} )}^{- 7 / 2}\end{matrix} \begin{matrix}{b}_{0} = 3 ( {\overline{x}}_{i}^{2} + {\overline{y}}_{i}^{2} + {\overline{z}}_{i}^{2} ) {\overline{x}}_{ij} {\overline{y}}_{ij} \\ {b}_{1} = 3 ( {\overline{x}}_{i}^{2} + {\overline{y}}_{i}^{2} + {\overline{z}}_{i}^{2} ) {\overline{y}}_{ij} {\overline{z}}_{ij} \\ {b}_{2} = 3 ( {\overline{x}}_{i}^{2} + {\overline{y}}_{i}^{2} + {\overline{z}}_{i}^{2} ) {\overline{x}}_{ij} {\overline{z}}_{ij} \\ {b}_{3} = 15 {\overline{x}}_{ij} {\overline{y}}_{ij} {\overline{z}}_{ij} \\ {b}_{4} = 3 {\overline{y}}_{ij} ( 4 {\overline{x}}_{ij}^{2} - {\overline{y}}_{ij}^{2} - {\overline{z}}_{ij}^{2} ) \\ {b}_{5} = 3 {\overline{z}}_{ij} ( 4 {\overline{x}}_{ij}^{2} - {\overline{y}}_{ij}^{2} - {\overline{z}}_{ij}^{2} ) \\ {b}_{6} = 3 {\overline{x}}_{ij} ( - {\overline{x}}_{ij}^{2} + 4 {\overline{y}}_{ij}^{2} - {\overline{z}}_{ij}^{2} ) \\ {b}_{7} = 3 {\overline{z}}_{ij} ( - {\overline{x}}_{ij}^{2} + 4 {\overline{y}}_{ij}^{2} - {\overline{z}}_{ij}^{2} ) \\ {b}_{8} = 3 {\overline{x}}_{ij} ( - {\overline{x}}_{ij}^{2} - {\overline{y}}_{ij}^{2} + 4 {\overline{z}}_{ij}^{2} ) \\ {b}_{9} = 3 {\overline{y}}_{ij} ( - {\overline{x}}_{ij}^{2} - {\overline{y}}_{ij}^{2} + 4 {\overline{z}}_{ij}^{2} )\end{matrix}$$

$$\begin{matrix}{c}_{0} = sin {θ}_{j} cos {ϕ}_{j} \\ {c}_{1} = sin {θ}_{j} sin {ϕ}_{j} \\ {c}_{2} = cos {θ}_{j} \\ {c}_{3} = cos {θ}_{j} cos {ϕ}_{j} \\ {c}_{4} = cos {θ}_{j} sin {ϕ}_{j} \\ {c}_{5} = sin {θ}_{j} ,\end{matrix}$$

and yielding the submatrix

[symbol]

$${J}_{ij} = {a}_{0} \text{?} .$$

$$\text{?} \text{indicates text missing or illegible when filed}$$

Assigning the remaining repeating elements of this submatrix to three additional local variables,

_{yz}=b₃c₀+b₇c₁+b_{g}c₂

a_{zz}=b₅c₀+b₃c₁+b₈c₂

a_{xy}=b₄c₀+b₆c₁+b₃c₂,

gives Jacobian submatrices of the form

[symbol]

$$\text{?}$$

$$\text{?} \text{indicates text missing or illegible when filed}$$

noting the variable a₆ multiplied to all of the elements of the matrix.

When computing the full Jacobian matrix, it can be fastest to compute these submatrices column-wise first, noting that the orientation information contained in variables c₀ through c₅ is common through all submatrices corresponding to an individual magnet, and thus allowing the sines and cosines to be computed just once per magnet.

### 6.1.2 Common Subexpression Elimination of the Magnetic Field Prediction Error Calculation

Now applying the same strategy as above to the magnetic field prediction calculation, as shown in FIG. 30, while as much as possible preserving similar notation as in the calculations above, subexpressions for the magnetic field prediction error were calculated as

[symbol]

$${a}_{0} = {\overline{x}}_{ij}^{2}$$

$${a}_{1} = {\overline{y}}_{ij}^{2}$$

$${a}_{2} = {\overline{z}}_{ij}^{2}$$

$${a}_{7} = {a}_{0} + {a}_{1} + {a}_{2}$$

$${a}_{8} = {\overline{m}}_{j} {{a}_{7}}^{- 5 / 2}$$

$${c}_{2} = {θ}_{j}$$

$${c}_{3} = {ϕ}_{j}$$

$${c}_{4} = sin {c}_{3}$$

$${c}_{3} = cos {c}_{3}$$

$${c}_{5} = sin {c}_{2}$$

$${c}_{2} = cos {c}_{2}$$

$${c}_{0} = {c}_{5} {c}_{3}$$

$${c}_{1} = {c}_{5} {c}_{4}$$

$${d}_{3} = 3 ( {\overline{x}}_{ij} {c}_{0} + {\overline{y}}_{ij} {c}_{1} + {\overline{z}}_{ij} {c}_{2} )$$

$${d}_{0} = {\overline{x}}_{ij} {d}_{3} - {a}_{7} {c}_{0}$$

$${d}_{1} = {\overline{y}}_{ij} {d}_{3} - {a}_{7} {c}_{1}$$

$${d}_{2} = {\overline{z}}_{ij} {d}_{3} - {a}_{7} {c}_{2} ,$$

giving relevant local variables equal to the expressions

[symbol]

$${a}_{8} = \frac{{\overline{m}}_{j}}{{( {\overline{x}}_{ij}^{2} + {\overline{y}}_{ij}^{2} + {\overline{z}}_{ij}^{2} )}^{\frac{5}{2}}}$$

$${d}_{0} = 3 {\overline{x}}_{ij} ( {\overline{x}}_{ij} sin ( {θ}_{j} ) cos ( {ϕ}_{j} ) + {\overline{y}}_{ij} sin ( {θ}_{j} ) sin ( {ϕ}_{j} ) + {\overline{z}}_{ij} cos ( {θ}_{j} ) ) - ( {\overline{x}}_{ij}^{2} + {\overline{y}}_{ij}^{2} + {\overline{z}}_{ij}^{2} ) sin ( {θ}_{j} ) cos ( {ϕ}_{j} )$$

$${d}_{1} = 3 {\overline{y}}_{ij} ( {\overline{x}}_{ij} sin ( {θ}_{j} ) cos ( {ϕ}_{j} ) + {\overline{y}}_{ij} sin ( {θ}_{j} ) sin ( {ϕ}_{j} ) + {\overline{z}}_{ij} cos ( {θ}_{j} ) ) - ( {\overline{x}}_{ij}^{2} + {\overline{y}}_{ij}^{2} + {\overline{z}}_{ij}^{2} ) sin ( {θ}_{j} ) sin ( {ϕ}_{j} )$$

$${d}_{2} = 3 {\overline{z}}_{ij} ( {\overline{x}}_{ij} sin ( {θ}_{j} ) cos ( {ϕ}_{j} ) + {\overline{y}}_{ij} sin ( {θ}_{j} ) sin ( {ϕ}_{j} ) + {\overline{z}}_{ij} cos ( {θ}_{j} ) ) - ( {\overline{x}}_{ij}^{2} + {\overline{y}}_{ij}^{2} + {\overline{z}}_{ij}^{2} ) cos ( {θ}_{j} ) .$$

The magnetic field prediction error was then computed as

$${E}_{ix} = ( {G}_{x} + {\sum }_{{j}^{′} = 0}^{{j}^{′} = M} {d}_{0} {a}_{8} ) - {\tilde{B}}_{ix} \qquad ( 6.2 a )$$

$${E}_{iy} = ( {G}_{y} + {\sum }_{{j}^{′} = 0}^{{j}^{′} = M} {d}_{1} {a}_{8} ) - {\tilde{B}}_{iy} \qquad ( 6.2 b )$$

$${E}_{iz} = ( {G}_{z} + {\sum }^{{j}^{′} = M} {d}_{2} {a}_{8} ) - {\tilde{B}}_{iz} \qquad ( 6.2 c )$$

Following a similar strategy to that above, it can be best to compute the prediction of the magnetic field contributions from each magnet first, to avoid recomputing sines and cosines. Magnetic field contributions from each magnet were summed to vector components as they were computed, and then, afterwards, the disturbance field and measured field were added and subtracted, respectively.

### 6.2 Practical Notes

Common notation was deliberately used between the subexpressions for the magnetic field prediction vector and the subexpressions for the Jacobian matrix. As indicated by this common notation, further common subexpression elimination can be performed by combining the magnetic field prediction vector and Jacobian matrix calculations, further reducing the latency of the optimization. However, for simplicity, in this work, it was chosen not to combine these two calculations, because the optimization algorithm employed, the Levenberg-Marquardt algorithm, does not request the optimization function and its derivative the same number of times. To combine the calculations, the source code of this algorithm can be altered to ensure that the magnetic field prediction values are not overwritten whenever only the derivative is requested. Use with other optimization algorithms or other hardware may make combining these two calculations more clearly advantageous.

In addition, parallelization was found to be inefficient with the hardware used in this work, but the computation of the magnetic field prediction error Jacobian matrix is a parallel process. With specialized hardware, the Jacobian matrix can be calculated using up to MN different processing streams, and the part of the magnetic field prediction calculation before the summation step can also be parallelized along with the Jacobian matrix. Further, an inspection of the common subexpression elimination flow diagrams (see FIGS. 28-30) reveals that these calculations are well-suited for implementation on an ASIC (application-specific integrated circuit) or an FPGA (field-programmable gate array) using hardware description language, on which full parallelization without delays from overhead can be possible.

A significant part of the time delay in these calculations is due to the sine and cosine operations, so any sizeable advance in the speed of these operations can have a non-negligible effect on the speed of the tracking algorithm. Noting that the sine and cosine operations in these subexpressions are both performed on both θⱼ and φⱼ, and further noting that there is some redundancy of information between sine and cosine, the computational latency was able to be

further reduced by using the sincos( ) function [21]. This modification was implemented after the results of the Taylor Reference, but before the experiments described in Example 1 herein. There are other methods for combining the sine and cosine operations, but the use is again hardware-specific. For example, an implementation of CORDIC tested with our algorithm was found to be slow on macOS, but CORDIC may be the best choice on another platform.

Depending upon the particular optimization implementation, the Jacobian matrix can be transposed or vectorized from its current structure before usage with an optimization algorithm. With this current implementation, it was found to be fastest to store the Jacobian directly as a vector instead of using multiple dimensions, allowing storage and access with fewer operations. The final step of the common subexpression elimination techniques discussed above can be implemented in a manner which directly produces this vectorization of the Jacobian using a sparse matrix multiplied by a vector containing the orientation information, but without extremely specialized hardware, this would likely not be as efficient as computing each of the elements as discussed above, unnecessarily requiring additional memory and increasing the number of common subexpressions required.

While every effort was made to minimize the number of local variables in each function, in the case of the products boco and boci, where these operations are only repeated when tracking the dipole strength, these duplicate subexpressions were allowed to remain. This decision was just for simplicity, noting that low latencies were only required when tracking magnetic dipoles of known size. Similarly, when tracking magnets with reduced dimensionality (e.g., position tracking on a surface with fixed orientation), some of the intermediate variables used above need not be computed, and the particular common subexpression elimination method can be adapted to the particular degrees of freedom used.

### 6.3 Improvement Via Newton's Method

It is worthy of considerable focus to investigate the use of second-order optimization using Newton's Method of optimization. In Chapter 2 and Chapter 3, we used the Levenberg-Marquardt algorithm to minimize the sum of the squared magnetic field prediction error,

$$S = {\sum }_{i = 1}^{N} {\sum }_{{*=}_{\{ x , y , z \}}}^{} {E}_{i *}^{2} , \qquad ( 6.3 )$$

subject to the tracking parameters

p=(x₁,y₁,z₁,θ₁,ϕ₁,\ddot{m}₁, . . . ,x_{M},y_{M},z_{M},θ_{M},ϕ_{M},\ddot{m}_{M},Gₓ,G_{y},G_{z}).

The first order partial derivative of S with respect to the jth tracking parameter is given by

$${( ∇ S )}_{j} = 2 {\sum }_{i = 1}^{N} {\sum }_{{*=}_{\{ x , y , z \}}}^{} {E}_{i *} \frac{\partial {E}_{i *}}{\partial {p}_{j}} , \qquad ( 6.4 )$$

which is the (doubled) dot product of the jth column of the Jacobian matrix of E with E, where E=(E₁ₓ,E_{1y},E_{1z}, . . . ,E_{Nx},E_{Ny},E_{Nz})|. More succinctly, these partial derivatives can be expressed as the vector

∇S=2J^{T}E. (6.5)

This vector ∇S was the gradient of the optimization used for magnet tracking with the LevenbergMarquardt algorithm.

To use Newton's Method, the second order partial derivatives of S, corresponding to the matrix elements of the Hessian of S, is also provided

$${H}_{jk} = 2 {\sum }_{i = 1}^{N} {\sum }_{{*=}_{\{ x , y , z \}}}^{} ( \frac{\partial {E}_{i *}}{\partial {p}_{j}} \frac{\partial {E}_{i *}}{\partial {p}_{k}} + {E}_{i *} \frac{{\partial }^{2} {E}_{i *}}{\partial {p}_{j} \partial {p}_{k}} ) . \qquad ( 6.6 )$$

The summed first term of this equation is simply the dot product of the jth column of the Jacobian matrix of E with the kth column of the Jacobian matrix of E. The summed second term is an element of the tensor contraction of the Hessian of E with E. In other words, if the elements of the Hessian of E are given by

$${{H}^{′}}_{i * jk} = \frac{{\partial }^{2} {E}_{i *}}{\partial {p}_{j} \partial {p}_{k}} , \qquad ( 6.7 )$$

then (6.6) can be rewritten as

$${H}_{jk} = 2 {\sum }_{i = 1}^{N} {\sum }_{{*=}_{\{ x , y , z \}}}^{} ( \frac{\partial {E}_{i *}}{\partial {p}_{j}} \frac{\partial {E}_{i *}}{\partial {p}_{k}} + {E}_{i *} {{H}^{′}}_{i * jk} ) , \qquad ( 6.8 )$$

and the Hessian of S is given by the matrix equation

H=2(J^{T}J+EH′), (6.9)

where H′ is the Hessian of E, and EH′ represents the tensor contraction of the Hessian of E with E. Representing this tensor contraction as H=EH′ and defining the “half-Hessian” of S as H̅ Δ ⅓ H gives

H⁻=J^{T}J+H. (6.10)

Borrowing scale invariance from the Levenberg-Marquardt algorithm, the equation for the vector step size δ can then be represented as

(H⁻+λdiag(H⁻))δ=J^{T}E. (6.11)

Upon solving (6.11) for δ, the tracking parameters can then be iteratively updated as

pₜ₊₁=pₜ−δ (6.12)

Newton's method, when combined with the scale invariance parameter from the Levenberg-Marquardt algorithm as in (6.11), can exhibit increased stability in the magnet tracking problem. Further, it is anticipated that the additional computational delay required to calculate the Hessian elements can be compensated for by a reduced step count needed to reach convergence.

Common subexpression elimination (and parallelization, with applicable hardware) can substantially accelerate the calculation of the Hessian matrix, making the solving of the matrix equation (6.11) the dominant driver of time-delay. The use of common subexpression elimination can be applied simultaneously to the calculation of the Hessian matrix, Jacobian matrix, and magnetic field predictions. Further, the simplicity of executing the matrix algebra for Newton's method can open the potential to implement the entire algorithm on an FPGA. As such, the time delay of the tracking algorithm can be substantially further reduced.

### 7. Further Advances

To address the high magnet tracking time delays in state-of-the-art multiple-magnet tracking, a high-speed multiple-magnetic-target tracking algorithm was developed, with latencies low enough to enable reflexive prosthesis control. Then, finding limitations to the use of magnet tracking in portable applications, a disturbance rejection algorithm was developed which can be implemented entirely in software. To address the need for a framework to calibrate multiple magnetic field sensors at once, previous ellipsoid fitting techniques were extended and improved to adapt them specifically to multi-sensor calibration. Lastly, seeing a need for a minimally-invasive technology to accurately track muscle lengths and speeds in real time, a new technology, magnetomicrometry was developed to meet this need, and the technology was validated by tracking muscle lengths in an animal model.

A framework for the next generation of magnetomicrometry is provided, which comes with a host of additional improvements. The orientation and position calibration discussed in Section 4 herein and the improved magnetic dipole strength measurement discussed in Sections 3 and 4 can enable new sensor geometries and improved accuracy. For example, magnetic field sensors can be placed on a curved array (see FIG. 34), stacked in multiple layers, or both, without a need to precision assemble or optically measure the positions and orientations of the components. Assembly of a curved array, such as shown in FIG. 34, can, for instance, be performed by fabricating the circuit on a flexible board and using an adhesive to affix it to a rigid non-planar geometry. For example, epoxy can be used to attach flexible magnetic field sensing circuitry flush with an outer surface of a prosthetic socket, ensuring that the magnetic field sensors are placed as proximal to the magnetic beads as possible. This flexible magnetic field sensing circuitry can take a form similar to the multiple sensor grids fabricated for the experiments described in Section 1 and Example 1 herein, or it can take a form similar to an LED tape light strip, allowing it to be continuously wrapped around the prosthetic socket.

An example of a non-planar sensing array 1100 is shown in FIG. 34. As illustrated, the non-planar sensing array is curved, but other geometries are possible, including irregularly curved and angled devices. The array 1100 includes a plurality of sensing elements 1101 and can be positioned against anatomy, or in or on a prosthetic device that encloses an anatomy (not shown for clarity), in which magnetic targets 101 are implanted.

A method of assembling a non-planar sensing array includes fabricating a plurality of sensors (e.g., magnetic field sensors) on a flexible circuit board and affixing the flexible circuit board to a non-planar and rigid substrate (e.g., a prosthetic socket).

### 7.1 Sensing of Systems of Permanent Magnets Wherein the Relative Positions of Multiple Sensing Arrays are Tracked via Inertial Measurement Units

One advantage of using multiple magnetic beads within a single tissue (e.g., muscle) is that this sensing modality is not affected by movements of the sensing array relative to the muscle. When multiple sensing arrays are used, these sensing arrays can be rigidly fixed in position and orientation relative to one another to ensure that movement of the arrays does not result in sensing noise. For example, the multiple arrays can be fixed to a carbon fiber prosthetic socket. There may arise situations, however, in which it is best to not have a rigid element holding the arrays together. For example, for an amputee with an osseo-integrated prosthesis, a socket is not needed. In this instance, it may be better to have the sensing arrays attached independently. Methods and systems providing for independent, flexible attachment of multiple sensing arrays to track systems of magnetic beads are provided. In such systems and methods, not all magnetic beads need be sensed by all sensing arrays in order for tracking to occur of each bead relative to the system of beads.

Such methods and systems provide for the use of multiple sensor arrays and in which the arrays are tracked relative to one another.

An example system in shown in FIG. 31. In this example, more than one sensing array is included (e.g., sensing arrays 1208a, 1208b, each with a plurality of sensing elements 1201 configured to detect a state of at least one magnetic target at a tissue). Each sensing array 1208a, 1208b includes a position sensor 1210 (e.g., an inertial measurement unit, accelerometer, etc.) to monitor a position and orientation of each sensing array (hereafter referred to as the pose of the sensing array) relative to the at least one other array. A controller 1230 in a wired or wireless arrangement with the sensing arrays can be configured to receive position data of the sensing arrays.

The pose of each array can be compared against the pose of the other arrays. For example, one of the arrays can be designated as the reference array, with a global coordinate system defined to be that seen by the reference array. The pose of all other sensing arrays can then be calculated with respect to the reference array. Regardless of how the coordinate system is defined, all sensing arrays of the system can be brought into a common coordinate system. More particularly, the positions and orientations of each of the magnetic field sensors on each sensing array are then calculated in this new common coordinate system using the calculated relative pose of the sensing arrays, and the magnetic beads are then tracked with respect to the common coordinate system.

An example method is shown in FIG. 32. The method 1300 includes the sensor arrays beginning at a known position and orientation relative to one another (1302). Translation acceleration and angular velocity as sensed by an inertial measurement unit (IMU) of each array can be used to monitor the relative poses of the sensor arrays (1304) and, in parallel, each array can track at least a subset of the magnetic targets included in or at an anatomy (1306). The relative poses of the sensor arrays and tracking parameters for at least a subset of magnets tracked by each array are combined to determine relative positions of all magnets in the system relative to one another (1308).

The sensor arrays can be connected to one another in a manner that restricts their relative degrees of freedom and enables the remaining relative degrees of freedom to be sensed with greater simplicity. For example, one or more hinges can be used for two or more sensor arrays, with one or more potentiometers to sense the angle between the sensor arrays. In another example, one or more linear bearings can be used to reduce the relative movement of the arrays to a single degree of freedom, and the relative positions can be measured via a distance sensor, such as an infrared sensor. In either of the above implementations, a flex sensor, IMU, or other sensing strategy can be used to determine the relative poses of the sensor arrays.

### 7.4 Preferred Sensor Magnetic Bead Tracking

When tracking magnetic beads, there can be a tradeoff between accuracy and tracking speed. From a hardware perspective, this tradeoff can be driven by the number of sensors that are used when tracking and the number of magnets that are tracked simultaneously by a shared set of sensors. This section describes an example system and method in which this tradeoff can be overcome by tracking multiple magnets for different sets of sensors that are themselves monitored with respect to their relative locations. The following describes a method whereby this tradeoff can be overcome by adjusting how the magnetic field sensors are used in the tracking.

A method is described for the strategic use of subsets of sensor data which can result in improved tracking accuracy. By using highly relevant subsets of the sensor data for tracking, the tracking accuracy and the size of the tracking volume can be improved upon with reduced tracking requirements.

For example, with the system 1200, the controller 1230 can further be configured to determine a distance and difference in orientation between each of the at least two sensing arrays and at least one magnetic target. Optionally, at least one magnetic target can be associated with one of the at least two sensor arrays based upon the determined distances and orientations.

### 7.4.1 Nearest Sensor Ranking for Magnetic Bead Tracking, Via Tracked Magnetic Bead Location

In this example, the tracked location of the magnetic beads is used to rank the relevance of the sensors. For each bead at each timepoint, the location of the bead as tracked at the previous timepoint is used (or the previous position, velocity, orientation, angular velocity, and/or accelerations of the bead can be used to predict the current timepoint parameters), the distance between the bead and the sensor is calculated, and all sensors are ranked according to their distance from the tracked location of the bead, with shorter distances imparting higher ranking. For efficiency, the square of the distance may instead be used to perform this ranking. To rank the sensors when multiple beads are used, the ranking can be performed for each of the beads independently and the two rankings can be merged. There are many different ways that these rankings can be merged, but one example of this merging iterates between the two lists in compiling a new list while ensuring that each independent list makes an equal (or off-by-one) number of contributions to the merged list. To track the magnets using a subset of the set of sensors, the subset is chosen giving priority to higher ranked sensors first.

### 7.4.2 Strongest Sensor Ranking for Magnetic Bead Tracking, Via Estimated Magnetic Bead Magnetic Field Contribution at Each Sensor

While the example described in Section 7.4.1 can provide a simple, efficient rule of thumb for selecting a subset of sensors, it may not yield the highest accuracy. Instead, the sensors that are the most relevant with respect to each magnetic bead can be considered to be the sensors which measure the greatest magnetic fields. The example method and system described herein addresses this relevance by ranking the sensors based upon the magnetic field contribution of each of the magnetic beads. In this example, for each bead at each timepoint, the location of the bead as tracked at the previous timepoint is used, and the predicted magnetic field is calculated along each axis of each sensor. For each magnetic bead, the sensors are ranked according to the strength (magnitude) of the magnetic field as measured at the sensors. This ranking can be performed using the magnitude of the vector magnetic field predicted from all three axes at once, or it can be performed ranking all axes of all sensors independently (resulting in tracking which does not necessarily include all three components of each sensor used in the tracking). When multiple magnetic beads are tracked, the independent rankings can be combined as described in Section 7.4.1. Again, in this instance, the subset of the set of sensors chosen can be selected based upon priority, with higher ranked sensors being selected first.

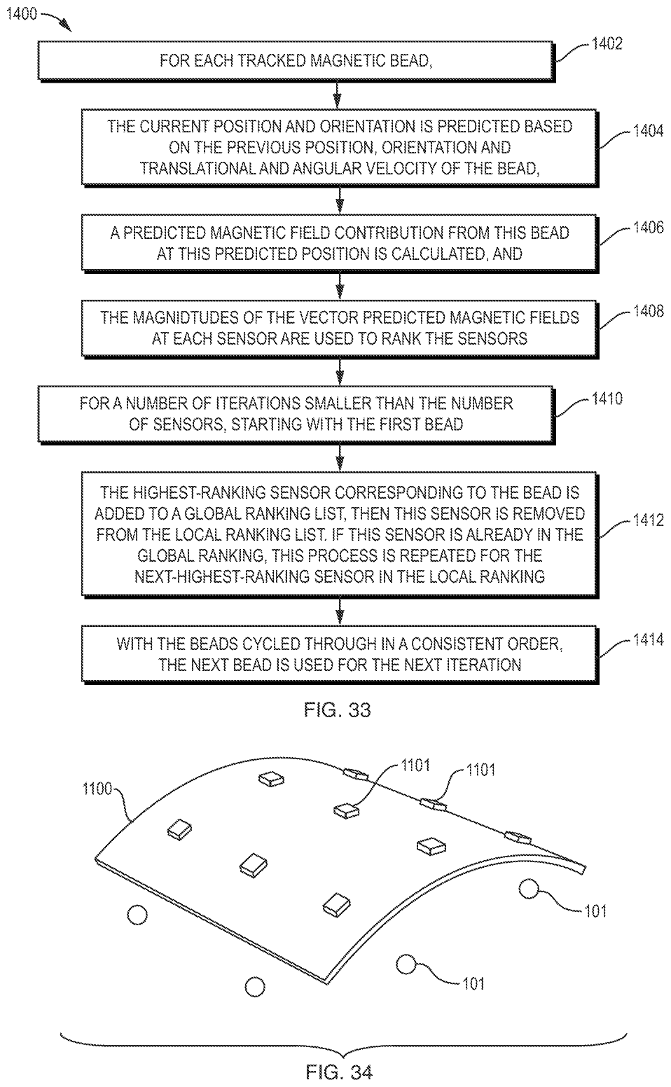

A flowchart is shown in FIG. 33 illustrating an example method 1400 of ranking for magnetic bead tracking. The method includes, for each tracked magnetic bead (1402), predicting the current position and orientation based on the previous position, orientation, and translational and angular velocity of the bead (1404). The method further includes calculating a predicted magnetic field contribution from the considered bead at the predicted position (1406) and ranking the sensors of the system based on the magnitudes of the vector predicted magnetic fields at each sensor (1408). For a number of iterations smaller than the number of sensors, and starting with the first bead (1410), the highest-ranking sensor corresponding to the bead is added to a global ranking list and the sensor is removed from the local ranking list (1412). With the beads cycled through in a consistent order, a next bead is used for a next iteration (1414).

### 7.5 Further Considerations

In addition to improved geometry, improved tracking methods such as the use of the Hessian to implement Newton's method (as discussed in Section 6.3 herein) can allow for further increased tracking stability, and can further reduce magnet tracking time delay. The use of specialized hardware, such as a hybrid or full FPGA approach, can substantially reduce the time delay required for magnet tracking. Increased tracking stability and reduced tracking time delay further allow tracking more magnets simultaneously in real time, allowing additional flexibility in sensor positioning when tracking many magnets at once.

To further maintain both accuracy and time delay while extending tracking range over a larger volume, an algorithm that makes use of only the nearest sensors to each magnet can be used. This could, for instance, first use all sensors to determine the initial magnet locations, and then use the tracked magnet positions to sort the sensors according to magnet proximity. Alternatively, the signal-to-noise ratio for each sensor can be ranked based on each magnet's predicted magnetic field.

In theory, an instability should exist when the magnet is aligned along its reference axis. Though no practical issues were encountered around vertical orientation in this work, if instability along the reference axis is determined to be non-negligible, the use of quaternions may not be sufficient to solve this issue due to the symmetry of the magnet. However, using an alternative reference axis whenever the magnet is oriented near the primary reference axis can solve this issue, if it exists. The use of the Hessian matrix via Newton's method of optimization can be sufficient to remove any potential of the instability.

No clear method for estimating the error of magnet tracking on-the-fly has been proposed, but it is possible to use the final Jacobian matrix of each tracking step to estimate the error of the current magnet position and orientation estimate using propagation of uncertainty. Because the Jacobian matrix gives the change in magnetic field error with respect to position and orientation parameters, each element of the Jacobian matrix can be inverted to give the error in position and orientation parameters that would be induced by an error in the magnetic field measurement. Assuming that the magnetic field error has a known, normal distribution, these inverted Jacobian matrix elements can then be weighted by the standard deviation of this normal distribution and averaged to compute an estimate of the error. If more precision in the error estimate is needed (including in the asymmetry of the error estimate about the estimated pose), the Hessian elements can be used to further refine this error estimate using the inverse of the second derivative elements. These error estimates can then be used as part of a Kalman filter to, in turn, increase the tracking accuracy.

All sensors are inherently noisy. When magnets are tracked using an optimization algorithm, the algorithm performs the equivalent function of smoothing out this noise, combining the data from multiple sensors to determine a single estimate for each parameter. Assuming that the magnetic field sensor noise is normally distributed (that the noise is chiefly Johnson and flicker noise), and that the error in a single position or orientation parameter that would be caused by one individual component of a three-axis sensor in isolation is linearly related to the error in sensing, it would then be expected that there exists a corresponding error distribution for each tracking parameter, comprising errors which correspond to each component of each three axis sensor. Though the optimization used for magnet tracking approaches the magnet parameter estimation holistically rather than individually, these theoretical individual tracking parameter error distributions can be teased out and one can then calculate their standard deviations and use these standard deviations as a proxy for error estimation of each of the parameters during each tracking step.

To determine how these individual error estimates can be calculated, we note that the Jacobian matrix in our magnet tracking algorithm provides the derivative of the magnetic field prediction, and that at the end of each tracking step we have available the estimated magnet parameters (e.g., x₁) and both the predicted magnetic field (e.g., B_{1(1)y}, referring to the predicted magnetic field contribution from the first magnet on the y-axis component of the first sensor) and measured magnetic field (e.g. \tilde{B}_{1y}) values for each component of each sensor. For a single component of a sensor and a single magnet parameter, then, when tracking a single magnet, we have for example, in point-slope form the equation

$${B}_{1 ( 1 ) y} - {\tilde{B}}_{1 y} = \frac{\partial {B}_{1 y}}{\partial {x}_{1}} ( {x}_{1} - {\tilde{x}}_{1 ( 1 y )} ) \qquad ( 7.1 )$$

where x^{\tilde{ }}_{1(1y)} is the magnet parameter as corrected using the measured magnetic field. If we then let

e_{x1(1y)}=x₁−x^{\tilde{ }}_{1(1y)} (7.2)

represent the estimated error in estimate of xi corresponding to the measured magnetic field B^{\tilde{ }}_{1y}, we have

$${e}_{x 1 ( 1 y )} = \frac{{B}_{1 ( 1 ) y} - {\tilde{B}}_{1 y}}{\frac{\partial {B}_{1 y}}{\partial {x}_{1}}} \qquad ( 7.3 )$$

and in generalizing these particular parameters to the context of multiple magnet tracking, we can subtract the predicted magnetic field contribution from all magnets other than the first magnet to retain validity of the equation as

$${e}_{x 1 ( 1 y )} = \frac{{B}_{1 ( 1 ) y} - ( {\tilde{B}}_{1 y} - ( {B}_{1 y} - {B}_{1 ( 1 ) y} ) )}{\frac{\partial {B}_{1 y}}{\partial {x}_{1}}} \qquad ( 7.4 )$$

$$= \frac{{B}_{1 y} - {\tilde{B}}_{1 y}}{\frac{\partial {B}_{1 y}}{\partial {x}_{1}}} \qquad ( 7.5 )$$

$$= \frac{{E}_{1 y}}{( \frac{\partial {B}_{1 y}}{\partial {x}_{1}} )} \qquad ( 7.6 )$$

This gives us a distribution of errors of our singled-out parameter corresponding to all of the sensor components, which distribution we can then use to better understand the accuracy of our parameter estimation. For instance, we can then calculation the standard deviation of the distribution of the estimated errors in the x₁ estimate as

$${e}_{x {1}_{UNWEIGHTED}} = \sqrt{\frac{{e}_{x 1 ( 1 x )}^{2} + {e}_{x 1 ( 1 y )}^{2} + {e}_{x 1 ( 1 z )}^{2} + ⋯ + {e}_{x 1 ( Nx )}^{2} + {e}_{x 1 ( Ny )}^{2} + {e}_{x 1 ( Nz )}^{2}}{3 N}} \qquad ( 7.7 )$$

A vector of all of the standard deviations in this unweighted form can be calculated via the generalized equation

$${e}_{UNWEIGHTED} = ( \frac{1}{3 N} {( J {°}^{- 2} )}^{T} E {°}^{2} ) {°}^{\frac{1}{2}} \qquad ( 7.8 )$$

where [symbol] represents element-wise (Hadamard) operations on the matrices. To account for the skewing that occurs in the values that are far away, the weighting

$${w}_{ij} = \frac{{r}_{ij}^{- 4}}{3 {\sum }_{i = 1}^{N} {r}_{ij}^{- 4}} \qquad ( 7.9 )$$

can be applied to each submatrix corresponding to i and j of the element-wise-squared-inverse Jacobian matrix instead of division by 3N.

In summary, to determine an estimate of the error for each of the tracking parameters, at the end of each tracking step, the Jacobian can be inverted element-wise and squared element-wise, weighted by the inverse fourth power of the distance from the magnets to the sensors to account for skewing, then multiplied by the element-wise squared magnetic field prediction error column-vector. The element-wise square root of the resulting column-vector should then be used in estimating the tracking parameter errors.

Even without these improvements, magnetomicrometry is well-positioned to have a tremendous impact in many fields. However, these next-generation modifications will serve to increase the robustness, speed, and accuracy of this tool, enabling it to have an even farther-reaching effect.

Additional descriptions of systems and methods relating to tracking of magnetic objects can be found in WO2019/074950, “Method for Neuromechanical and Neuroelectromagnetic Mitigation of Limb Pathology;” the teachings of which are incorporated herein in their entirety.

Additional descriptions of systems and methods relating to tracking of multiple targets, such as permanent magnets, can be found in the following publication: Cameron R Taylor, Haley G Abramson, and Hugh M Herr. Low-latency tracking of multiple permanent magnets. IEEE Sensors Journal, 19(23):11458-11468, 2019; the teachings of which are incorporated herein in their entirety.

### EXEMPLIFICATION

### Example 1

### Minimally-Invasive Muscle Tracking Using Permanent Magnets

An in-vivo turkey model with a prototype magnetomicrometry system was investigated to address biocompatibility, verify in-vivo tracking accuracy, and ensure long-term resistance of the implants to migration.

### Methods

All animal experiments were approved by the Institutional Animal Care and Use Committees at Brown University and the Massachusetts Institute of Technology.

For surgical implantation of the magnetic beads, four turkeys were placed on anesthesia under 3-4% isofluorane, then plucked on both legs and scrubbed with Povidone-Iodine 10% (PVP-I). Turkeys were then transferred to a hot pad, intubated and actively ventilated, while monitoring pulse oximetry, breathing rate, and body temperature. The alternate leg was then wrapped in a sterile covering, the perimeter of the surgical field was draped with sterile cloth, and the leg was sterilized with PVP-I. At each insertion site (the distal and proximal ends of the gastrocnemius and iliotibialis cranialis muscles), a 16 gauge needle and a thin pair of unopened surgical scissors were consecutively used to make an insertion channel smaller than the diameter of the magnet. The magnet was then press-fit into the end of a sterile hollow plastic tube, dipped in saline, and inserted into the channel using depth markings on the plastic tube for reference. A sterile wooden rod (longer than the plastic tube) was then guided fully into the bore of the plastic tube and used to gently, but firmly, hold the magnet in place while removing the plastic tube from the muscle. The wooden rod was then removed, and nonmagnetic forceps were used to suture the muscle closed at the insertion site using 6-0 nonabsorbable silk suture for the muscle and 4-0 Vicryl absorbable suture for the skin, after which Tegaderm (3M) transparent film dressing was applied to the skin around the insertion site.

For biocompatibility, all magnets (3-millimeter-diameter N35 neodymium-iron-boron spherical magnets, initially coated in nickel) were coated in parylene C (BJA Magnetics). Each magnet's strength was then measured and recorded, and the magnet was rinsed in 70% ethanol by volume (in distilled water) followed by three rinses with distilled de-ionized water. Each magnet was then placed in a labeled, vented container and sterilized using ethylene oxide, after which they were allowed 48 hours to degas before surgical implantation.

During surgical implantation, magnet pairs were inserted, with the aid of a sterile ruler, at various separation distances between approximately 20 and 70 millimeters. Immediately after surgical implantation and at time intervals (multiple weeks) after the implantation, computed tomography (CT) scans (Animage Fidex Veterinary CT Scanner) were used to monitor the distances between the beads. Turkeys were placed on anesthesia under 3-4% isofluorane, and for each leg, the turkey lay prone with the leg of interest flush with, centered on, and parallel to the scanning table, with the foot positioned as cranial and medial to the body as possible. Each leg was scanned separately to simplify positioning in the scanner and reduce the possibility of needing to repeat scans. In each CT scan, a reference object (an acrylic bar with magnets press-fit into two measured, pre-drilled holes) was included to ensure consistency in scale. A medical image viewer (Horos) was used to determine the three-dimensional positions of the magnetic beads in each muscle, and these positions were used to calculate the magnetic bead separation distances.

To track the magnets, two custom sensing boards, each with 48 LIS3MDL magnetic field sensors (STMicroelectronics) in a 6×8 grid spaced at 4.83 millimeters, were held together by a 3d-printed fixture (Connex 500, Stratasys) at 60 millimeters apart from board center to board center, forming a single, 96-magneticfield-sensor array (see FIGS. 3A-3B). Nylon nuts and bolts (McMaster-Carr) were used to secure the sensing boards to the fixture. A custom adapter board was used to connect a Teensy 3.6 microcontroller (PJRC) to the sensing boards using flexible flat cables (Molex), and on-board 4-to-16 line decoders (74HC154BQ, Nexperia) were used to individually enable magnetic field sensors for SPI communication (10 MHz clock).

The magnetic field at each of the sensors was measured with a sampling rate of 300 Hz. To minimize onboard magnetic field distortion, all capacitors used (Vishay) were MRI-safe. The tracking algorithm was run in real-time on a Macbook Air (13-inch, Early 2014) with 8 GB of RAM and an Intel i7 CPU running at 1.7 GHz.

To validate magnetomicrometry (see FIG. 4), for each of the legs of each of the four turkeys, the 96-magnetic-field-sensor array 250a was secured with veterinary tape to the outside of the turkey's leg over the magnetic bead pair 270 in the gastrocnemius muscle. With the turkey anesthetized (3-4% isofluorane), an electric motor 272 (Aurora Scientific 310B-LR) was used to apply a mechanical frequency sweep to the turkey's ankle (10-second exponential chirp from 0.7 to 7 Hz), with a spring 274 (surgical tubing) providing an opposing force. Throughout this frequency sweep of the ankle (and thus, of the passively-cycled gastrocnemius muscle), the magnetic field sensor array was used, as described in the Taylor Reference, to track the length of the gastrocnemius muscle using the distance between the magnetic beads in real time. For comparison, the distance between the magnetic beads, which are radio-opaque, was also simultaneously monitored via fluoromicrometry (stereo X-ray videofluoroscopy), with the X-ray sources 282 above the turkey and the image intensifiers 280 positioned below. All fluoromicrometry data was post-processed in XMALab, whenever possible automating the processing using 25% “threshold offset in percent,” manually performing tracking when reprojection error exceeded one pixel, and without performing any temporal filtering to smooth the data. Time syncing was used to perform initial alignment of the magnetomicrometry and fluoromicrometry curves, but due to inconsistency in the time sync signal from the X-ray system, optimization was used to fine-tune the temporal alignment of the data.

To investigate static tracking between magnetomicrometry and fluoromicrometry, for evaluating compatibility between the two technologies as well as sensing depth for the magnetomicrometry, two magnets were placed into a 1×10 LEGO plate at various known distances apart from one another, and the magnetic field sensor array was placed above the magnets at various sensing heights.

To then further investigate the presence of translational offsets between the sensor arrays, and to further investigate sensing depth, the two magnets were again placed into the 1×10 LEGO plate at 24 millimeters apart from one another, with a single magnetic field sensor board above the magnets at a finer interval of sensing heights.

Turkeys were labeled with identification cuffs by number and color, Green #5 (Turkey A), BlueGreen #2 (Turkey B), Yellow #1 (Turkey C), and Blue #6 (Turkey D).

### Results: Magnetomicrometry

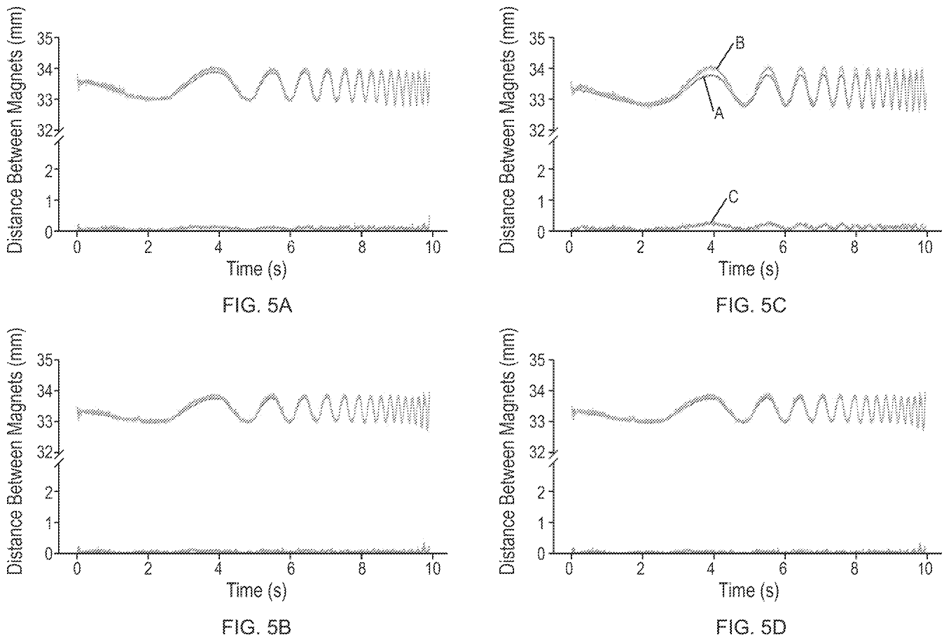

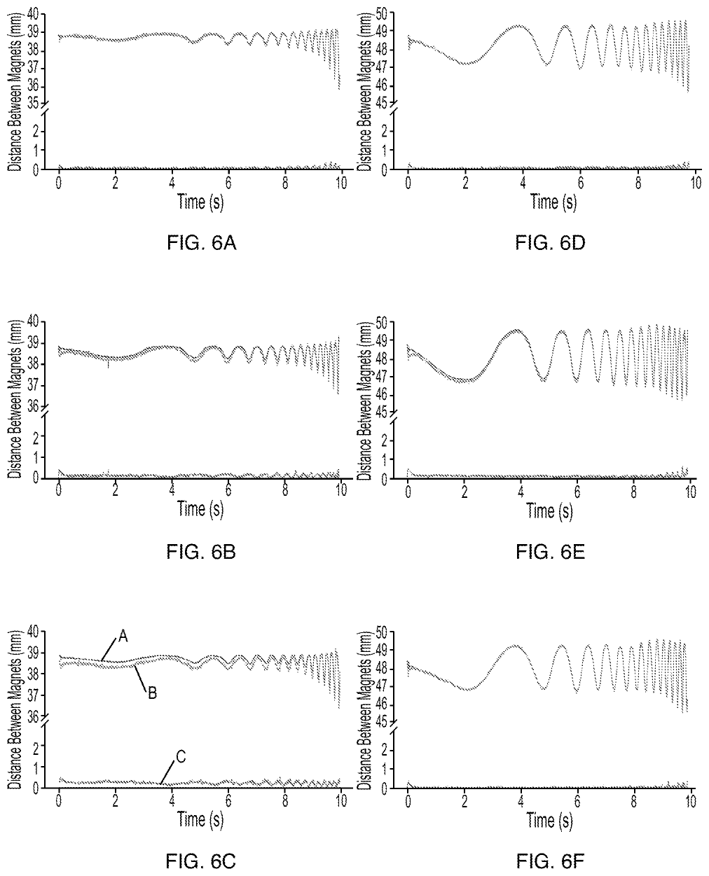

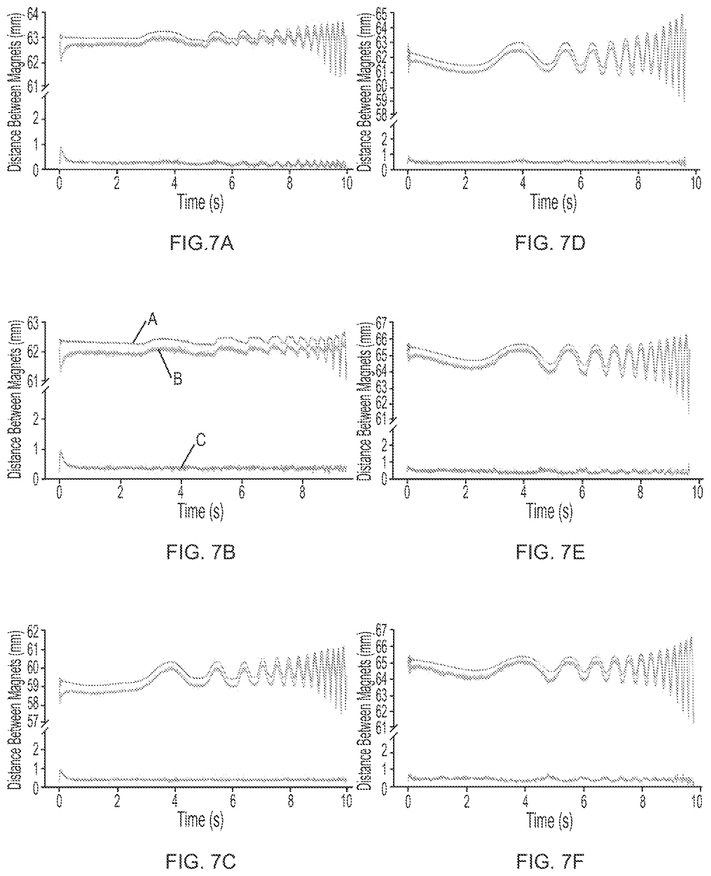

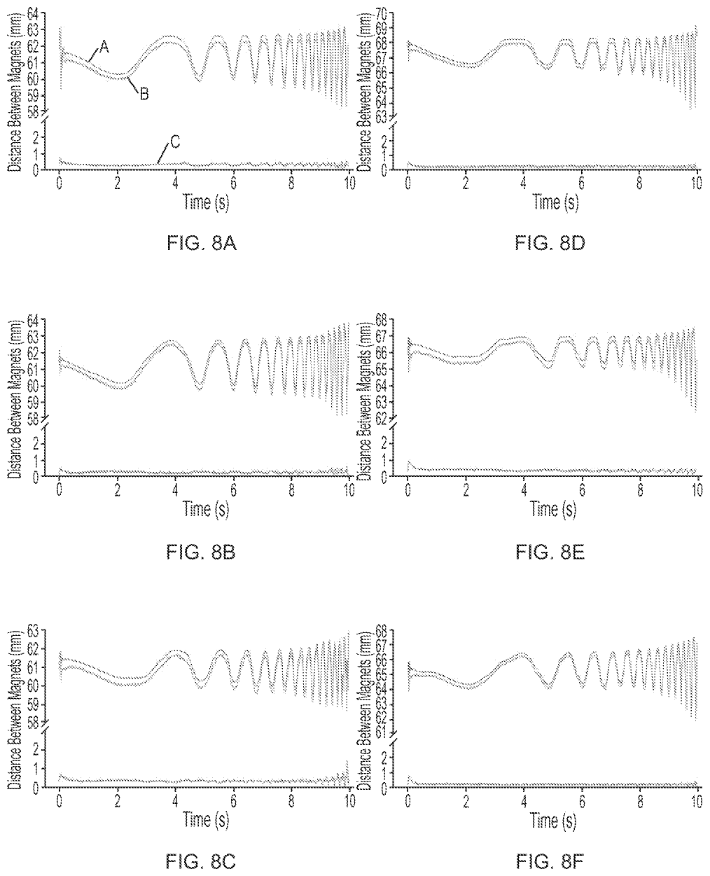

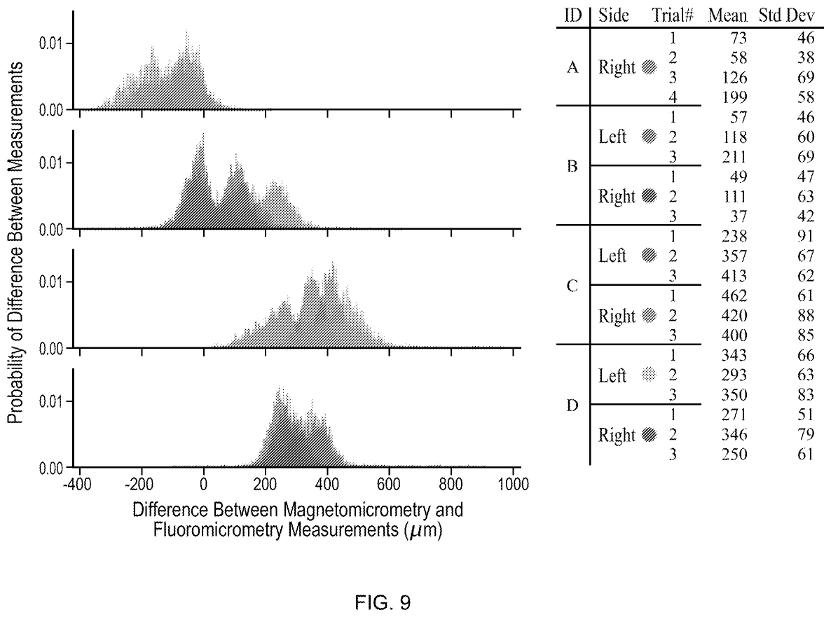

The results of the in-vivo turkey magnetomicrometry studies are shown in FIGS. 5-9, with FIGS. 5-8 showing the distance between the magnets over time throughout the frequency sweep for each trial of each leg of each turkey. The experiments corresponding to Turkey A (FIGS. 5A-D) represent frequency sweeps on the right leg only, because the magnetic bead pair in the left gastrocnemius of Turkey A migrated together, as described below with respect to long-term stability of the implants against migration. In the remaining turkeys, B through D (FIGS. 6A-F, 7A-F, and 8A-F), plots show the three frequency sweeps corresponding to each leg. In all plots, magnetomicrometry is shown against fluoromicrometry, as well as the absolute difference between them. In FIG. 9, the differences between magnetomicrometry and fluoromicrometry are given, in histogram form, for all of the results shown in FIGS. 5-8. Inspection of these figures shows a consistent offset between magnetomicrometry and fluoromicrometry for each set of trials for a given leg. This offset is most clearly demonstrated in the trials for Turkeys C and D. It is expected that this issue is due to misalignment of the magnetic field sensing boards, because they were attached to one another using a 3D-printed fixture and plastic screws. It is thus expected that these consistent offsets seen can be corrected with calibration or high-precision fixturing. As shown by the table in FIG. 9, the standard deviation of the differences is consistently below 100 micrometers, suggesting that with correction for positioning, the system is capable of at least this precision.

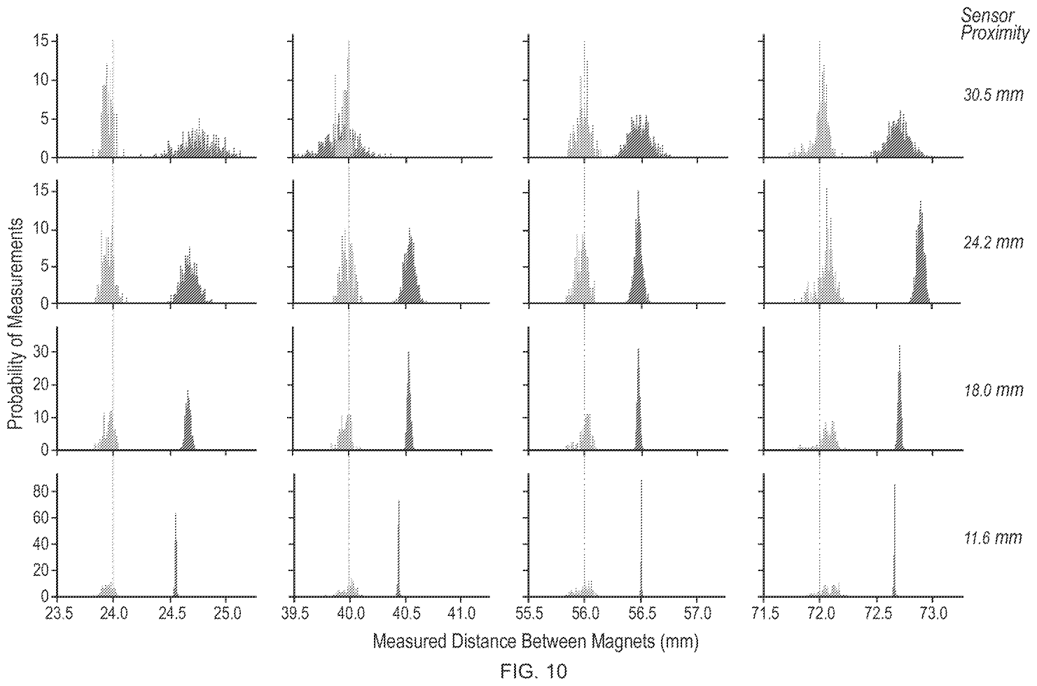

To further compare magnetomicrometry and fluoromicrometry in a controlled, static setting, ensuring that the two technologies did not interfere with one another and investigating the need for proximity of the sensors to the magnets, the magnetic field sensor array was set at various heights and the magnetic beads were separated to various distances, and both technologies were used to simultaneously measure the distances between the magnets. FIG. 10 shows these results, where the magnetic beads were placed at distances of approximately 24, 40, 56, and 72 mm from one another and the sensor was placed at various heights above the magnetic beads. Note that, with the magnetic field sensors close to the magnetic beads, magnetomicrometry has a measurement standard deviation that is significantly lower than fluoromicrometry, but as the magnetic field sensors are distanced from the beads, the magnetomicrometry measurement standard deviation approaches and then becomes larger than that of the fluoromicrometry measurement standard deviation. Also note that, again, with the exception of the 40 mm magnetic bead separation measured with a 30.5 mm sensor proximity, there is a consistent offset between the magnetomicrometry and fluoromicrometry measurements, again suggesting the need for precision fixturing or sensor position calibration.

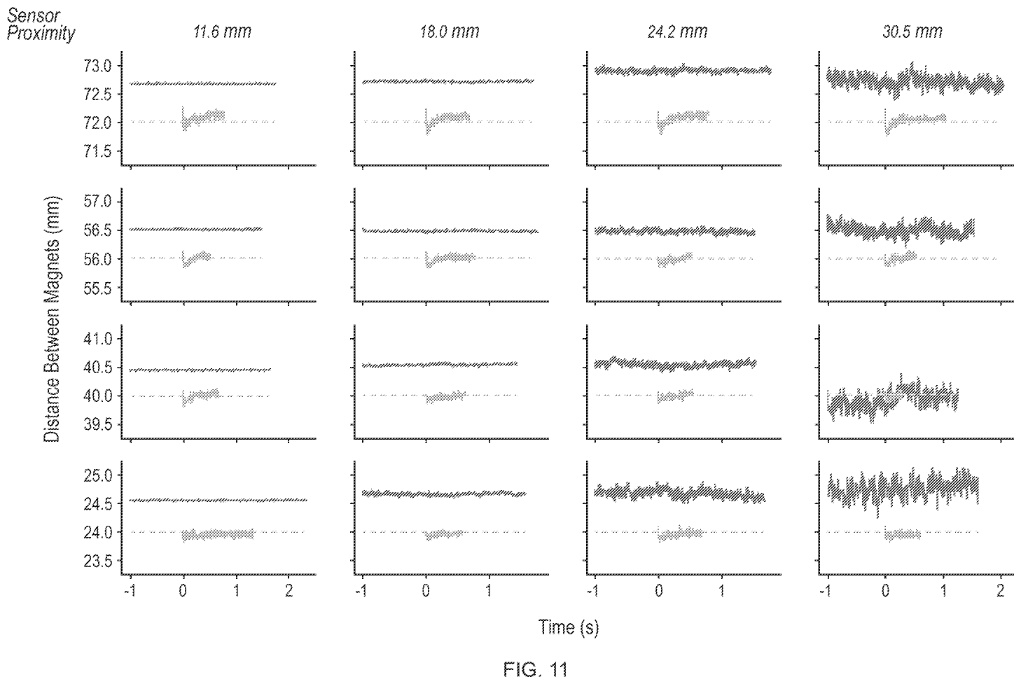

Visualizing this same data temporally (see FIG. 11), we find that the fluoromicrometry does not interfere with the magnetomicrometry measurements (the magnetomicrometry measurements are consistent before, during, and after the X-ray irradiation).

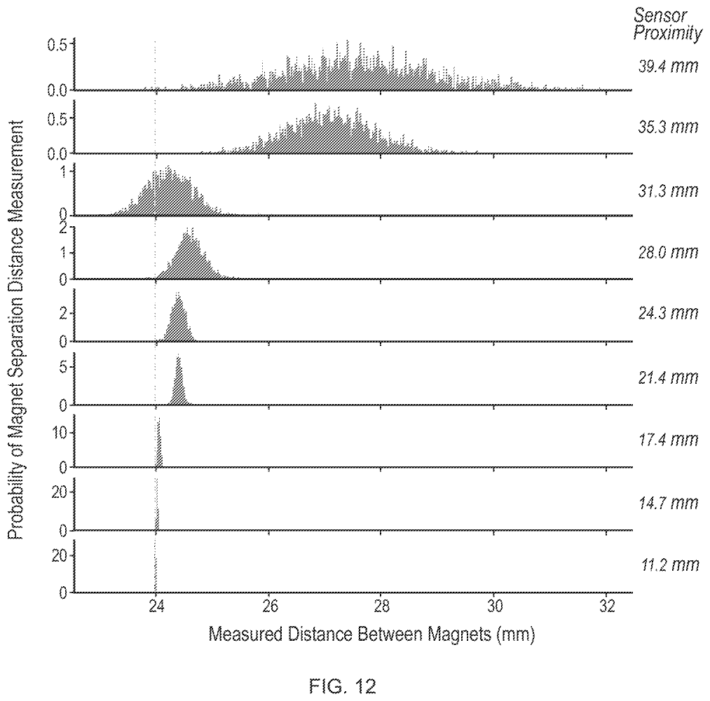

Finally, to further investigate the offset that was encountered between the magnetomicrometry and fluoromicrometry measurements, and to better understand accuracy at various magnetic field sensor proximities, the static experiment was repeated using just one single board (without fluoromicrometry) above magnetic beads spaced approximately 24 mm apart in the 1×10 LEGO plate (see FIG. 12 for the results). Using a single board ensures that the positions of the magnetic field sensors are all well-known relative to one another. In this experiment, separation distance measurements were found to have high-accuracy at near proximity and sub-millimeter accuracy continuing to a sensing distance of just over 30 mm, followed by a pronounced increase in error thereafter.

### Results: Long-Term Implant Stability Against Migration

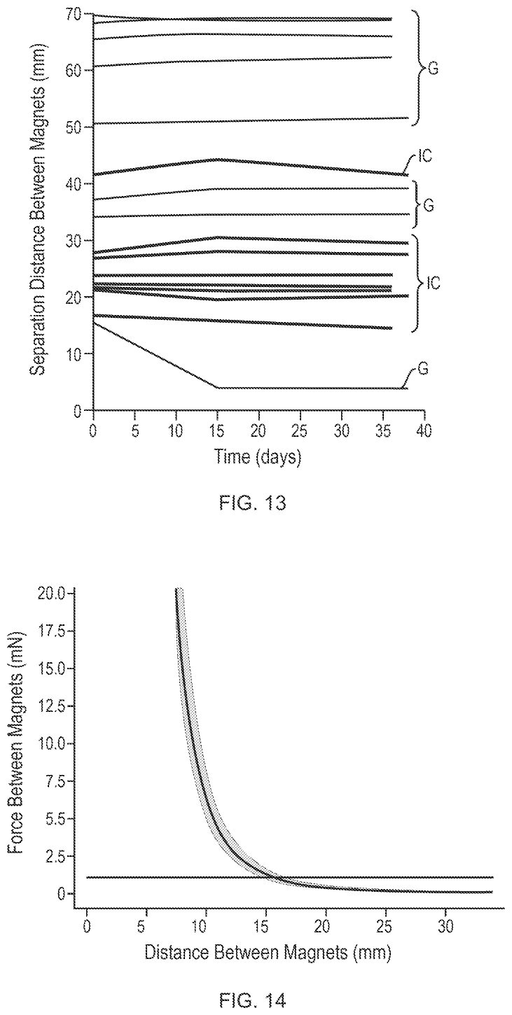

The computed tomography (CT) scans of the gastrocnemius and iliotibialis cranialis muscles were used to create a migration plot (see FIG. 13) showing the separation distances of the magnetic bead pairs over time. The magnetic bead pair that was implanted closest to one another, with an initial separation distance measured at 15.3 mm, did undergo migration, as can be seen by the 3 mm final separation of this pair in FIG. 13. The second closest magnet pair, with an initial separation distance measured at 16.7 mm, did not fully migrate. However, as the migration study is still ongoing at the time of this writing, this pair may yet experience additional migration. Upon observing all other magnetic bead pairs, it appears that the beads at longer separation distances are all resilient long-term to migration, suggesting magnetic beads should be implanted with separation distances of at least 20 mm or more.

### Discussion: Magnetomicrometry

The results presented above suggest that magnetomicrometry is viable for human prosthetic and exoskeletal control. The results presented above demonstrate sub-millimeter accuracy, and with improved sensor positioning or calibration, it appears that the accuracy resolution can be easily brought to well below 100 micrometers. Even in the absence of improved sensor positioning, the precision of the measurements is sufficient to faithfully track normalized muscle lengths and velocities in real time. In other words, when the geometry of an application permits, it is expected that comparable to superior accuracy can be achieved from magnetomicrometry as would be found in fluoromicrometry, but with the additional benefits of being portable, low-cost, and real-time.

The frequency sweep data that was collected was performed via passive cycling of the muscle via movement of the ankle joint, but larger excursions are expected when the muscle is actively cycled.

The data shown in FIG. 11, from the static measurement interference test, demonstrates that the process of fluoromicrometry does not interfere with the magnetomicrometry measurements. However, these results do not provide insight into whether the magnetomicrometry interferes with the fluoromicrometry.

In the single-board static magnetomicrometry experiments (see FIG. 12), the spread in the measurement (i.e., the standard deviation) can be explained simply by the normally-distributed Johnson and flicker noise, but the mean error cannot be explained in this same way, so the mean offset deserves additional investigation to determine the cause.

### Discussion: Long-Term Implant Stability Against Migration

Long-term implant stability depends on the properties of muscle tissue, the size and coating of the magnets, and the forces that the magnets exert on one another. The interaction between muscle tissue properties and the size and coating of the magnets on long-term stability is best empirically investigated, but the forces that magnets exert on one another is given by a simple mathematical model. Using equation (37) of [24] and assuming that the magnets in each pair of magnetic beads are aligned with one another, the magnitude of the force between two magnets in a magnet pair is given as:

$$\begin{matrix}F = ‖ \frac{3 {μ}_{0} {m}_{1} {m}_{2}}{4 π {d}^{4}} ( \hat{d} ( {\hat{m}}_{1} \cdot {\hat{m}}_{2} ) + {\hat{m}}_{1} ( \hat{d} \cdot {\hat{m}}_{2} ) + {\hat{m}}_{2} ( \hat{d} \cdot {\hat{m}}_{1} ) - \\ 5 \hat{d} ( \hat{d} \cdot {\hat{m}}_{1} ) ( \hat{d} \cdot {\hat{m}}_{2} ) ) ‖ \\ = \frac{3 {μ}_{0}}{2 π} \frac{{m}_{1} {m}_{2}}{{d}^{4}} ,\end{matrix} \qquad ( 1.1 )$$

where d is the separation distance between the magnets. Thus, with the above assumption on magnet alignment, Eqn. 1.1 can be used to determine an estimate of the force between the magnets. In Table 1.1, the magnet strengths as measured before implantation are compiled along with the standard deviation of these strength measurements and the minimum and maximum measured magnet separation distances after implantation.

TABLE 1.1
Implanted Magnetic Bead Pair Strengths and Separations

| | | | Magnetic Dipole Strength (mA · m²) | | | | | |
|---|---|---|---|---|---|---|---|---|
| | | | Magnet 1 | | Magnet 2 | | Separation (mm) | |
| Turkey ID | Side | Muscle | Strength | Std Dev | Strength | Std Dev | Max | Min |
| A | L | Gastroc | 10.3 | 2.0 | 10.4 | 0.6 | 15.3 | 3.6 |
| | | ITC | 9.0 | 0.5 | 9.0 | 0.5 | 21.2 | 19.4 |
| | R | Gastroc | 9.1 | 0.5 | 9.2 | 0.4 | 34.6 | 34.0 |
| | | ITC | 9.2 | 1.9 | 9.3 | 0.5 | 44.2 | 41.5 |
| B | L | Gastroc | 9.7 | 0.7 | 9.7 | 0.6 | 39.2 | 37.1 |
| | | ITC | 9.6 | 0.8 | 9.6 | 0.5 | 30.4 | 27.7 |
| | R | Gastroc | 9.4 | 0.4 | 9.4 | 0.4 | 51.6 | 50.5 |
| | | ITC | 9.4 | 0.4 | 9.6 | 0.3 | 27.9 | 26.7 |
| C | L | Gastroc | 10.2 | 0.5 | 10.2 | 0.5 | 66.5 | 65.6 |
| | | ITC | 8.1 | 1.5 | 10.1 | 1.4 | 21.5 | 21.0 |
| | R | Gastroc | 9.9 | 0.4 | 9.9 | 0.5 | 69.3 | 68.4 |
| | | ITC | 10.1 | 0.4 | 9.9 | 0.4 | 16.7 | 14.3 |
| D | L | Gastroc | 9.9 | 0.4 | 9.9 | 0.4 | 62.3 | 60.7 |
| | | ITC | 9.8 | 1.9 | 10.0 | 0.4 | 22.1 | 21.6 |
| | R | Gastroc | 10.0 | 0.5 | 10.0 | 0.4 | 69.7 | 68.9 |
| | | ITC | 9.8 | 1.4 | 10.1 | 1.0 | 23.8 | 23.6 |

It may be tempting to use Eqn. 1.1 with Table 1.1 to calculate a maximum allowable force between magnets. For instance, using the first row of the Table 1.1, corresponding to the magnetic bead pair that migrated, it is tempting to consider using the strength of the migrated magnet pair minus the measurement standard deviation for each magnet, along with the maximum measured separation distance of the migrated magnet pair, to determine a somewhat conservative estimate of maximum allowable force. This approach, however, would be flawed. Assuming a neodymium iron boron volumetric mass density of 7.5 g/cm³, a 3 mm diameter sintered neodymium iron boron magnet experiences a force due to gravity of approximately 1.040 mN. In contrast, the above calculation gives only 0.887 mN, significantly less than the force due to gravity on the magnet. Revisiting Eqn. 1.1, the force increases with the inverse fourth power of the distance between the magnetic beads, so, importantly, the magnetic beads experience a significantly greater force at smaller distances. For instance, now using just the magnetic dipole strengths from the first row of Table 1.1 with Eqn. 1.1, the force versus magnetic bead separation distance is shown in FIG. 14.

Noting this steep increase in force as the magnetic bead separation distance is reduced, it is essential to note that the minimum magnetic bead separation distance should take into account the minimum distance that the magnetic beads will come within one another under maximum contraction. As seen in the magnetomicrometry studies, the magnets come several millimeters closer to one another during passive cycling. It is expected that the magnetic beads come even closer to one another during active flexion of the muscles.

Finally, migration is also dependent upon both the magnets' strength and the magnets' geometry, so one can take into account tolerances in both of these parameters when creating specifications for safe magnet separation distances in tissue.

### BIBLIOGRAPHY

1. Ariel L Camp, Henry C Astley, Angela M Horner, Thomas J Roberts, and Elizabeth L Brainerd. Fluoromicrometry: a method for measuring muscle length dynamics with biplanar videofluoroscopy. Journal of Experimental Zoology Part A: Ecological Genetics and Physiology, 325(7):399-408, 2016.

2. Jared W Coburn, T J Housh, J T Cramer, J P Weir, J M Miller, T W Beck, M H Malek, and G O Johnson. Mechanomyographic time and frequency domain responses of the vastus medialis muscle during submaximal to maximal isometric and isokinetic muscle actions. Electromyography and clinical neurophysiology, 44(4):247-255, 2004.

3. John V Frangioni, Tao S Kwan-Gett, Lynn E Dobrunz, and Thomas A McMahon. The mechanism of low-frequency sound production in muscle. Biophysical journal, 51(5):775-783, 1987.

4. Wolfgang Kabsch. A solution for the best rotation to relate two sets of vectors. Acta Crystallographica Section A: Crystal Physics, Diffraction, Theoretical and General Crystallography, 32(5):922-923, 1976.

5. Wolfgang Kabsch. A discussion of the solution for the best rotation to relate two sets of vectors. Acta Crystallographica Section A: Crystal Physics, Diffraction, Theoretical and General Crystallography, 34(5):827-828, 1978.

6. K&J Magnetics. Neodymium magnet physical properties: Magnet summary table. http://web.archive.org/web/20200331161055/https://www.kjmagnetics. com/specs. asp. Accessed: 2020-03-31.

7. SM Magnetics. Magnetic materials & tables: Neodymium. https://smmagnetics. com/pages/magnetic-materials-tables. Accessed: 2020 Mar. 31, Archived at http://web.archive.org/web/20200331160201/https://cdn.shopify.com/s/files/1/1359/3085/files/Material_tables_on_website_-Neodymium_-_2.pdf?4920142403209868357.

8. Thomas A McMahon. “Chapter 1: Fundamental Muscle Mechanics”. Muscles, reflexes, and locomotion. Princeton University Press, 1984.

9. T Nara and W Ito. Moore—penrose generalized inverse of the gradient tensor in euler's equation for locating a magnetic dipole. Journal of Applied Physics, 115(17):17E504, 2014.

10. Takaaki Nara, Satoshi Suzuki, and Shigeru Ando. A closed-form formula for magnetic dipole localization by measurement of its magnetic field and spatial gradients. IEEE Transactions on Magnetics, 42(10):3291-3293, 2006.

11. Talat Ozyagcilar. Calibrating an eCompass in the presence of hard and soft-iron interference. Freescale Semiconductor Ltd, pages 1-17, 2012.

12. None

13. Timo Pylvanainen. Automatic and adaptive calibration of 3d field sensors. ^{\ddot{ }} Applied Mathematical Modelling, 32(4):575-587, 2008.

14. Thomas J Roberts, Richard L Marsh, Peter G Weyand, and C Richard Taylor. Muscular force in running turkeys: the economy of minimizing work. Science, 275(5303):1113-1115, 1997.

15. Craig Schroeder. Practical course on computing derivatives in code. In ACM SIGGRAPH 2019 Courses, pages 1-22. 2019.

16. S Tarantino, F Clemente, D Barone, M Controzzi, and C J S R Cipriani. The myokinetic control interface: tracking implanted magnets as a means for prosthetic control. Scientific reports, 7(1):1-11, 2017.

17. Cameron R Taylor, Haley G Abramson, and Hugh M Herr. Low-latency tracking of multiple permanent magnets. IEEE Sensors Journal, 19(23):11458-11468, 2019.

18. Arnold Magnetic Technologies. Neodymium iron boron magnet catalog: Sintered neodymium-iron-boron magnets; other properties. https://web.archive.org/web/20200513225014/https://www.arnoldmagnetics.com/wp-content/uploads/2019/06/Arnold-Neo-Catalog.pdf. Accessed: 2020 May 13.

19. Arnold Magnetic Technologies. Neodymium iron boron magnets: Available neo grades. http://web.archive.org/web/20200331161416/https://www.arnoldmagnetics. com/products/neodymium-iron-boron-magnets/. Accessed: 2020 Mar. 31.

20. D A Turner, I J Anderson, J C Mason, and M G Cox. An algorithm for fitting an ellipsoid to data. National Physical Laboratory, UK, 1999.

21. The UNIX and Linux Forums. Linux and unix man pages: Bsd library functions manual; sincos(3). https://www.unix.com/man-page/osx/3/_SINCOS/. Accessed: 2020 May 18.

22. R F Wiegert. Magnetic star technology for real-time localization and classification of unexploded ordnance and buried mines. In Detection and Sensing of Mines, Explosive Objects, and Obscured Targets XIV, volume 7303, page 73031U. International Society for Optics and Photonics, 2009.

23. W Wynn, C Frahm, P Carroll, R Clark, J Wellhoner, and M Wynn. Advanced superconducting gradiometer/magnetometer arrays and a novel signal processing technique. IEEE Transactions on Magnetics, 11(2):701-707, 1975.

24. Kar W Yung, Peter B Landecker, and Daniel D Villani. An analytic solution for the force between two magnetic dipoles. Physical Separation in Science and Engineering, 9(1):39-52, 1998.

25. Kathryn Ziegler-Graham, Ellen J MacKenzie, Patti L Ephraim, Thomas G Travison, and Ron Brookmeyer. Estimating the prevalence of limb loss in the united states: 2005 to 2050. Archives of physical medicine and rehabilitation, 89(3):422-429, 2008.

26. Martin, Jack A., et al. “Gauging force by tapping tendons.” Nature communications 9.1 (2018): 1-9.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of detecting muscle activation, comprising:
with a magnetic field sensor, detecting lateral vibration of a target implanted at a muscle or a tendon, the target comprising a magnetic material; and
estimating a level of muscle activation based on the detected lateral vibration.

2. The method of claim 1, further comprising estimating a muscle force based on the estimated level of muscle activation and a length and a velocity of the muscle.

3. The method of claim 1 or 2, wherein detecting lateral vibration includes detecting movements of the target in a range of about 10 μm to about 20 μm.

4. The method of any one of claims 1-3, wherein detecting lateral vibration includes detecting vibrational movement of the target at frequencies of greater than about 10 Hz.

5. The method of any one of claims 1-4, further comprising, with an accelerometer, detecting vibrational movement of the magnetic field sensor relative to the target.

6. The method of any one of claims 1-5, wherein detecting lateral vibration of a target includes detecting lateral vibrations of two or more targets disposed along an axis.

7. A method of estimating a physiological parameter of a muscle or tendon, comprising:
with a magnetic field sensor, detecting vibration of a target implanted at a muscle or tendon, the target comprising a magnetic material; and
estimating at least one of a muscle force and tendon force based on the detected vibration.

8. The method of claim 7, further comprising:
applying a perturbance to the target or to a muscle-tendon unit comprising the muscle and the tendon;
measuring a timing of the vibration of the target relative to a timing of the perturbance or relative to a timing of a vibration of one or more additional targets implanted at the muscle-tendon unit; and
estimating a speed of a shear wave or a compression wave in the muscle-tendon unit or surrounding tissue of the muscle-tendon unit based on the measured timing.

9. The method of claim 8, wherein applying a perturbance includes poking or vibrating the muscle-tendon unit or the surrounding tissue.

10. The method of claim 9, wherein the poking or vibrating includes actuating a magnetic bead affixed at the muscle tendon unit or the surrounding tissue by an applied magnetic field.

11. The method of claim 10, wherein the applied magnetic field is supplied by an electromagnet.

12. The method of claim 11, wherein the electromagnet is external to the body.

13. The method of claim 8, further comprising determining a physiological property of the muscle-tendon unit based on the estimated speed of the shear wave or the compression wave.

14. The method of claim 13, wherein the physiological property is stiffness of the muscle, the tendon, or the surrounding tissue.

15. A method of monitoring biomechanical motion, comprising:
disposing at least one target on a subject at a location associated with a joint of the subject;
with a magnetic field sensor array, detecting a change in state of the at least one target relative to the magnetic field sensor array, another target disposed on the subject, or a combination thereof; and
determining a state of the joint based on the detected change in state of the at least one target.

16. A method for determining one or more of three sensor position parameters and three sensor orientation parameters for each of the sensors in a sensor array, comprising:
placing at least one target in at least one known location relative to a sensor array, whereby a signal from the at least one target at the sensors is detected, and recording at least one measurement of the signal at each of the sensors for each placement of the one or more targets;
estimating one or more parameters from the group consisting of x-position, y-position, z-position, yaw, pitch, and roll, of each of the sensors;
estimating any unknown state parameters of the at least one target;
providing a constant value for a magnetic dipole weight state parameter of the at least one target for each of the measurements;
calculating predicted values of the signal at each of the sensors for each of the measurements given the one or more estimated sensor parameters, the estimated target state parameters, and the provided constant magnetic dipole weight state parameter;
computing a prediction error in the predicted values of the signal with reference to the values of the signals detected at the sensors;
calculating a prediction error Jacobian matrix by analytically computing elements of the prediction error Jacobian matrix with respect to the estimated parameters of the sensors for each measurement; and
determining from the prediction error and the prediction error Jacobian matrix a state of the parameters of the sensors.

17. A method of calibrating a magnetic field sensor array, comprising:
three-dimensionally rotating a magnetic field sensor array in a uniform magnetic field, the magnetic field sensor array comprising a plurality of sensors;
recording data from each of the plurality of sensors;
calculating a non-rotating transformation of the recorded data using an ellipsoid fit of the data for each of the plurality of sensors;
calculating a scaling factor of each of the plurality of sensors based on relative dimensions of the ellipsoid fit;
applying the calculated non-rotating transformation and calculated scaling factor to obtain a transformed, scaled dataset for each of the plurality of sensors; and
rotating the obtained transformed, scaled datasets to align, thereby determining a relative orientation of each of the plurality of sensors to calibrate the magnetic field sensor array.

18. An implantable target for magnetic tracking, comprising:
a base structure comprising a magnetic or magnetizable material; and
a shell structure disposed about the base structure and comprising layers of nickel, copper, gold, and parylene C.

19. The implantable target of claim 18, wherein the layers of the shell structure are arranged from the base structure in order of nickel, copper, nickel, gold, and parylene C.

20. The implantable target of claim 18 or 19, wherein the gold layer of the shell structure has a thickness of at least about 5 μm.

21. The implantable target of any one of claims 18-20, wherein the parylene C layer of the shell structure has a thickness of at least about 25 μm.

22. The implantable target of any one of claims 18-21, wherein the magnetic or magnetizable material comprises neodymium iron boron and dysprosium.

23. An insertion device for an implantable target, comprising:
a cannula;
a cartridge configured to position an implantable target at the cannula; and
a pushrod receivable in the cannula and configured to push the implantable target from the cartridge through the cannula for delivery to a tissue, the cannula and the pushrod comprising a nonmagnetic material.

24. The insertion device of claim 23, wherein a distal end of the pushrod is of a complementary geometry to the implantable target to prevent damage to a shell structure of the target during delivery.

25. The insertion device of claim 23, wherein a distal surface of the pushrod comprises a spring structure to prevent damage to a shell structure of the target during delivery.

26. The insertion device of any one of claims 23-25, further comprising a mount coupling the cartridge to the cannula, wherein the cartridge is configured to retain a plurality of targets and is rotatable about the mount to position each of the plurality of targets at the cannula.

27. An insertion system, comprising:
a plurality of the implantable targets of claim 18; and
the insertion device of claim 23, the plurality of implantable targets being deliverable by the insertion device.

28. A wearable shielding assembly, comprising:
an array of sensors configured to detect a state change of at least one magnetic target implanted at a tissue;
a wearable receptacle within which the array of sensors is disposed; and
a geometrically-reconfigurable material disposed about or integral with the wearable receptacle and configured to provide magnetic shielding to the array of sensors.

29. The assembly of claim 28, wherein the geometrically-reconfigurable material is flexible.

30. The assembly of claim 28, wherein the geometrically-reconfigurable material comprises a plurality of ferromagnetic tiles.

31. The assembly of claim 30, wherein the ferromagnetic tiles are releasably connectable to one another.

32. The assembly of claim 28, wherein the geometrically-reconfigurable material comprises a ferromagnetic mesh.

33. The assembly of claim 28, wherein the geometrically-reconfigurable material is rigidly affixed after application to the wearable receptacle.

34. A method of tracking one or more permanent magnets, comprising:
detecting a signal from each of the one or more permanent magnets;
calculating an analytically-derived Hessian matrix with respect to the detected signals; and
determining a state of each of the one or more permanent magnets based on the calculated Hessian matrix.

35. The method of claim 34, further comprising:
calculating a predicted magnetic field value for each of the one or more permanent magnets; and
calculating a Jacobian matrix with respect to the detected signals, wherein determining the state of each of the one or more permanent magnets is further based on the calculated predicted magnetic field values and the calculated Jacobian matrix, and wherein calculation of the Hessian matrix, the Jacobian matrix, and the predicted magnetic field values is performed simultaneously using common subexpression elimination.

36. The method of claim 34, wherein the calculation of the Hessian matrix is parallelized.

37. A method of assembling a non-planar sensing array, comprising:
fabricating a plurality of sensors on a flexible circuit board; and
affixing the flexible circuit board to a non-planar and rigid substrate.

38. The method of claim 37, wherein the substrate is a prosthetic socket.

39. The method of claim 37 or 38, wherein the sensors are magnetic field sensors.

40. The method of any one of claims 37-39, wherein the flexible circuit board is a long strip.

41. A system, comprising:
at least two sensor arrays, each sensor array configured to detect a state of at least one magnetic target at a tissue; and
at least one position sensor associated with at least one of the at least two sensor arrays and configured to detect a position and orientation of the associated sensor array relative to the other of the at least two sensor arrays.

42. The system of claim 41, wherein the at least one position sensor is an inertial measurement unit disposed at the associated sensor array.

43. The system of claim 41 or 42, further comprising a controller configured to:
determine a distance and difference in orientation between each of the at least two sensor arrays and the at least one magnetic target; and
associate the at least one magnetic target with one of the at least two sensor arrays based upon the determined distances and orientations.

44. The system of any one of claims 41-43, wherein the at least one magnetic target is implanted at the tissue.

45. A system for detecting muscle activation, comprising:
a magnetic field sensor configured to detect lateral vibration of at least one target implanted at a muscle or a tendon, the at least one target comprising a magnetic material; and
a controller configured to estimate a level of muscle activation based on the detected lateral vibration.

46. The system of claim 45, wherein the controller is further configured to estimate a muscle force based on the estimated level of muscle activation and a length and a velocity of the muscle.

47. The system of claim 45 or claim 46, further comprising an accelerometer at the magnetic field sensor and configured to detect vibrational movement of the magnetic field sensor relative to the target.

48. The system of any one of claims 45-47, wherein the at least one target comprises two or more targets disposed along an axis.

49. A system for estimating a physiological parameter of a muscle or tendon, comprising:
a magnetic field sensor configured to detect vibration of at least one target implanted at a muscle or a tendon, the at least one target comprising a magnetic material; and
a controller configured to estimate at least one of a muscle force, a tendon force, and a muscle-tendon unit force based on the detected vibration.

50. The system of claim 49, wherein the controller is further configured to estimate a speed of a shear wave or a compression wave in the muscle, the tendon, a muscle-tendon unit comprising the muscle and the tendon, or surrounding tissue of the muscle-tendon unit based on a measured timing of a vibration of the at least one target relative to a timing of a perturbance applied to the target or relative to one or more additional targets implanted at the muscle or tendon.
